SEQUENCE LISTING (252 sequences)

SEQ ID NO: 1 - protein, 288 aa, Homo sapiens
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLW

SEQ ID NO: 2 - protein, 30 aa, Homo sapiens
PTPCVPAECFDLLVRHCVACGLLRTPRPKP

SEQ ID NO: 3 - protein, 76 aa, Homo sapiens
MRRGPRSLRGRDAPAPTPCVPAECFDLLVRHCVACGLLRTPRPKPAGASSPAPRTALQPQESVGAGAGEAALPLPG

SEQ ID NO: 4 - protein, 69 aa, Homo sapiens
RRGPRSLRGRDAPAPTPCVPAECFDLLVRHCVACGLLRTPRPKPAGASSPAPRTALQPQESVGAGAGEA

SEQ ID NO: 5 - protein, 60 aa, Homo sapiens
RGPRSLRGRDAPAPTPCVPAECFDLLVRHCVACGLLRTPRPKPAGASSPAPRTALQPQES

SEQ ID NO: 6 - protein, 49 aa, Homo sapiens
DAPAPTPCVPAECFDLLVRHCVACGLLRTPRPKPAGASSPAPRTALQPQ

SEQ ID NO: 7 - protein, 39 aa, Homo sapiens
DAPAPTPCVPAECFDLLVRHCVACGLLRTPRPKPAGASS

SEQ ID NO: 8 - protein, 136 aa, synthetic construct
RGPRSLRGRDAPAPTPCVPAECFDLLVRHCVACGLLRTPRPKPAGASSPAPRTALQPQESGGGGSGGGGSGGGGSRGPRSLRGRDAPAPTPCVPAECFDLLVRHCVACGLLRTPRPKPAGASSPAPRTALQPQESG

SEQ ID NO: 9 - protein, 227 aa, Homo sapiens
EMGTADLGPSSVPTPTNVTIESYNMNPIVYWEYQIMPQVPVFTVEVKNYGVKNSEWIDCINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKG

SEQ ID NO: 10 - protein, 182 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 11 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 12 - protein, 173 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 13 - protein, 164 aa, Homo sapiens
SGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 14 - protein, 409 aa, Homo sapiens
KNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 15 - protein, 217 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 16 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 17 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 18 - protein, 126 aa, Homo sapiens [sequence omitted]

SEQ ID NO: 19 - protein, 186 aa, Homo sapiens [sequence omitted]

SEQ ID NO: 20 - protein, 120 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 21 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 22 - protein, 332 aa, Homo sapiens
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIG

SEQ ID NO: 23 - protein, 522 aa, Homo sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 24 - protein, 213 aa, Homo sapiens; identical to SEQ ID NO: 16

SEQ ID NO: 25 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 26 - protein, 237 aa, Homo sapiens
GSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 29 - protein, 227 aa, Homo sp.
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 227 aa, Homo sapiens
DKTHTCPPCPAPELLGGPSVFLFPPKPKDTLMISRTPEVTCVVVDVSHEDPEVKFNWYVDGVEVHAAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 227 aa, Homo sapiens
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, Homo sapiens; identical to SEQ ID NO: 28

SEQ ID NO: 33 - protein, 227 aa, Homo sapiens
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 227 aa, Homo sapiens
DKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 36 - protein, 227 aa, Homo sapiens; identical to SEQ ID NO: 31

SEQ ID NO: 37 - protein, 227 aa, Homo sapiens
KYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCMVSHEALHNHYTQKSLSLSPGK

SEQ ID NO: 39 - protein, 243 aa, Homo sapiens
NVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 227 aa, Homo sapiens; identical to SEQ ID NO: 27

SEQ ID NO: 41 - protein, 244 aa, Homo sapiens
NVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 227 aa, Homo sapiens
DKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 5 aa, synthetic construct
GGGGS

SEQ ID NO: 44 - protein, 19 aa, synthetic construct
LGGGGSGGGGSGGGGSAAA

SEQ ID NO: 45 - protein, 22 aa, Homo sapiens
LSGGGGSGGGGSGGGGSGGGGS

SEQ ID NO: 46 - protein, 18 aa, synthetic construct
LAEAAAKEAAAKEAAAKE

SEQ ID NO: 47 - protein, 20 aa, synthetic construct
LAEAAAKEAAAKEAAAKAAA

SEQ ID NO: 48 - protein, 25 aa, synthetic construct
LAEAAAKEAAAKEAAAKEAAAKAAA

SEQ ID NO: 49 - protein, 33 aa, synthetic construct
LAEAAAKEAAAKAAANGSLAEAAAKEAAAKAAA

SEQ ID NO: 50 - protein, 46 aa, synthetic construct
AEAAAKEAAAKEAAAKEAAAKALEAEAAAKEAAAKEAAAKEAAAKA

SEQ ID NO: 51 - protein, 30 aa, synthetic construct
DKTHTCPPCPAPELLGGPSVFLFPPKPKDT

SEQ ID NO: 52 - protein, 29 aa, synthetic construct
DKTHTSPPSPAPELLGGPSVLFPPKPKDT

SEQ ID NO: 53 - protein, 18 aa, synthetic construct
DKTHTCPPCPAPELLGGP

SEQ ID NO: 54 - protein, 18 aa, synthetic construct
DKTHTSPPSPAPELLGGP

SEQ ID NO: 55 - protein, 14 aa, synthetic construct
DKTHTCPPCPAPEL

SEQ ID NO: 56 - protein, 14 aa, synthetic construct
DKTHTSPPSPAPEL

SEQ ID NO: 57 - protein, 39 aa, synthetic construct
DKTHTSPPSPAPELLGGPNGSDKTHTSPPSPAPELLGGP

SEQ ID NO: 58 - protein, 23 aa, synthetic construct
EPKSCDKTHTCPPCPAPELLGGP

SEQ ID NO: 59 - protein, 23 aa, synthetic construct
EPKSSDKTHTSPPSPAPELLGGP

SEQ ID NO: 60 - protein, 20 aa, synthetic construct
ESKYGPPCPSCPAPEFLGGP

SEQ ID NO: 61 - protein, 20 aa, synthetic construct
ESKYGPPSPSSPAPEFLGGP

SEQ ID NO: 62 - protein, 28 aa, synthetic construct
PAPAPAPPAPAPAPPAPAPAPPAPAPAP

SEQ ID NO: 63 - protein, 40 aa, synthetic construct
APKPAPEPKPAPKPAPEPKPAPKPAPEPKPAPKPAPEPKP

SEQ ID NO: 64 - protein, 36 aa, synthetic construct
GSAGSAAGSGEFGSAGSAAGSGEFGSAGSAAGSGEF

SEQ ID NO: 65 - protein, 15 aa, synthetic construct
GGGGSLVPRGSGGGG

SEQ ID NO: 66 - protein, 15 aa, synthetic construct
GGGSGGGSNTSGGGS

SEQ ID NO: 67 - protein, 11 aa, synthetic construct
GGGSNTTGGGS

SEQ ID NO: 68 - protein, 11 aa, synthetic construct
GGGSNASGGGS

SEQ ID NO: 69 - protein, 23 aa, synthetic construct
GGGGSGGGGSNTTGGGGSGGGGS

SEQ ID NO: 70 - protein, 4 aa, synthetic construct
GGGS

SEQ ID NO: 71 - protein, 23 aa, synthetic construct
GGGGSGGGGSNTTGGGGSGGGGS

SEQ ID NO: 72 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 73 - protein, 687 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 74 - protein, 773 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 75 - protein, 762 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEMGTADLGPSSVPTPTNVTIESYNMNPIVYWEYQIMPQVPVFTVEVKNYGVKNSEWIDCINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKG

SEQ ID NO: 76 - protein, 628 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKERGPRSLRGRDAPAPTPCVPAECFDLLVRHCVACGLLRTPRPKPAGASSPAPRTALQPQESLAEAAAKEAAAKEAAAKERGPRSLRGRDAPAPTPCVPAECFDLLVRHCVACGLLRTPRPKPAGASSPAPRTALQPQES

SEQ ID NO: 77 - protein, 550 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESGGGGSGGGGSRGPRSLRGRDAPAPTPCVPAECFDLLVRHCVACGLLRTPRPKPAGASSPAPRTALQPQES

SEQ ID NO: 78 - protein, 518 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNA

SEQ ID NO: 79 - protein, 689 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 80 - protein, 578 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNA

SEQ ID NO: 81 - protein, 439 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 82 - protein, 525 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 83 - protein, 611 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 84 - protein, 548 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 85 - protein, 499 aa, synthetic construct
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 86 - protein, 541 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 87 - protein, 433 aa, synthetic construct
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 88 - protein, 755 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKELAEAAAKEAAAKESYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 89 - protein, 784 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKELAEAAAKEAAAKEAAAKELNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSERQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 90 - protein, 775 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 91 - protein, 509 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPGASSPAPRTALQPQESVGAGAGEAALPLPGLLLAEAAAKEAAAKEAAAKELQMAGQCSQNEYFDSLLHACIPCQLRCSSNTPPLTCQRYCNASVTNSVKGTNA

SEQ ID NO: 92 - protein, 424 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKELQMAGQCSQNEYFDSLLHACIPCQLRCSSNTPPLTCQRYCNASVTNSVKGTNA

SEQ ID NO: 93 - protein, 487 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKELQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNA

SEQ ID NO: 94 - protein, 542 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNA

SEQ ID NO: 95 - protein, 542 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 96 - protein, 618 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKERGPRSLRGRDAPAPTPCVPAECFDLLVRHCVACGLLRTPRPKPGASSPAPRTALQPQESVGAGAGEAALPLPGLLGGGGSGGGGSLQMAGQCSQNEYFDSLLHACIPCQLRCSSNTPPLTCQRYCNASVTNSVKGTNA

SEQ ID NO: 97 - protein, 484 aa, synthetic construct
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKELQMAGQCSQNEYFDSLLHACIPCQLRCSSNTPPLTCQRYCNASVTNSVKGTNA

SEQ ID NO: 98 - protein, 516 aa, synthetic construct
EMGTADLGPSSVPTPTNVTIESYNMNPIVYWEYQIMPQVPVTVEVKNYGVKNSEWIDCINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNA

SEQ ID NO: 99 - protein, 506 aa, synthetic construct
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNALAEAAAKEAAAKEAAAKAAARGPRSLRGRDAPAPTPCVPAECFDLLVRHCVACGLLRTPRPKPAGASSPAPRTALQPQESVGAGAGEAALPLPGLL

SEQ ID NO: 100 - protein, 540 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKAAARGPRSLRGRDAPAPTPCVPAECFDLLVRHCVACGLLRTPRPKPAGASSPAPRTALQPQESVGAGAGEAALPLPGLL

SEQ ID NO: 101 - protein, 867 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSERQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 102 - protein, 606 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 103 - protein, 611 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKLAEAAAKEAAAKEAAAKAAARGPRSLRGRDAPAPTPCVPAECFDLLVRHCVACGLLRTPRPKPAGASSPAPRTALQPQESVGAGAGEAALPLPGLL

SEQ ID NO: 104 - protein, 610 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPDVFLFPPKPKDTLMISRTPEVTDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSGGGGSGGGGSNTTGGGGSGGGGSDGSFFLYSKLTVDKSRWQQGNVFSCSVMHEALHNHYTQKSLSLSPGKRGPRSLRGRDAPAPTPCVPAECFDLLVRHCVACGLLRTPRPKPAGASSPAPRTALQPQESVGAGAGEAALPLPGLL

SEQ ID NO: 105 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 106 - protein, 614 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHAAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSNTTGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 107 - protein, 610 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHAAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLSDGSFFLYSKLTVDKSRWQQGNVFSCSVMHEALHNHYTQKSLSLSPGKLAEAAAKEAAAKEAAAKAAARGPRSLRGRDAPAPTPCVPAECFDLLVRHCVACGLLRTPRPKPAGASSPAPRTALQPQESVGAGAGEAALPLPGLL

SEQ ID NO: 108 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 109 - protein, 616 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWEPKSSDKTHTCPSCSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKAAARGPRSLRGRDAPAPTPCVPAECFDLLVRHCVACGLLRTPRPKPAGASSPAPRTALQPQESVGAGAGEAALPLPGLL

SEQ ID NO: 110 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 111 - protein, 606 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 112 - protein, 611 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 113 - protein, 613 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSNSTGGGGSGGGSRGPRSLRGRDAPAPTPCVPAECFDLLVRHCVACGLLRTPRPKPAGASSPAPRTALQPQESVGAGAGEAALPLPGLL

SEQ ID NO: 114 - protein, 618 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSSDKTHTSPSSSAPEAAGGPSVFLRGPRSLRGRDAPAPTPCVPAECFDLLVRHCVACGLLRTPRPKPAGASSPAPRTALQPQESVGAGAGEAALPLPGLL

SEQ ID NO: 115 - protein, 620 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTSPPSPAPELLGGPSVFLFPPKPKDRGPRSLRGRDAPAPTPCVPAECFDLLVRHCVACGLLRTPRPKPAGASSPAPRTALQPQESVGAGAGEAALPLPGLL

SEQ ID NO: 116 - protein, 614 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEAEAAAKEAAAKEAAAKEAAAKARGPRSLRGRDAPAPTPCVPAECFDLLVRHCVACGLLRTPRPKPAGASSPAPRTALQPQESVGAGAGEAALPLPGLL

SEQ ID NO: 117 - protein, 606 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLVPRGSGGGGRGPRSLRGRDAPAPTPCVPAECFDLLVRHCVACGLLRTPRPKPAGASSPAPRTALQPQESVGAGAGEAALPLPGLL

SEQ ID NO: 118 - protein, 702 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSLVPRGSGGGGRGPRSLRGRDAPAPTPCVPAECFDLLVRHCVACGLLRTPRPKPAGASSPAPRTALQPQESVGAGAGEAALPLPGLL

SEQ ID NO: 119 - protein, 779 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSLVPRGSGGGGRGPRSLRGRDAPAPTPCVPAECFDLLVRHCVACGLLRTPRPKPAGASSPAPRTALQPQESVGAGAGEAALPLPGLLGGSGGHMGSGGRGPRSLRGRDAPAPTPCVPAECFDLLVRHCVACGLLRTPRPKPAGASSPAPRTALQPQESVGAGAGEAALPLPGLL

SEQ ID NO: 120 - protein, 560 aa, synthetic construct
SLRGRDAPAPTPCVPAECFDLLVRHCVACGLLRTPRPKPAGASSPAPRTALQPQESVGAGAGEAALPLPGLLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLVPRGSGGGGRGPRSLRGRDAPAPTPCVPAECFDLLVRHCVACGLLRTPRPKPAGASSPAPRTALQPQESVGAGAGEAALPLPGLLGGSGGHMGSGGSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQV

SEQ ID NO: 121 - protein, 526 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 122 - protein, 828 aa, synthetic construct
HAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTGGGGSGGGGSGGGGSMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 123 - protein, 466 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPEGGGGSGGGGSKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 124 - protein, 798 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 125 - protein, 572 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKGGGGSGGGGSFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 126 - protein, 829 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 127 - protein, 596 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 128 - protein, 672 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRGPRSLRGRDAPAPTPCVPAECFDLLVRHCVACGLLRTPRPKPAGASSPAPRTALQPQESVGAGAGEAALPLPGLLGGGGSRGPRSLRGRDAPAPTPCVPAECFDLLVRHCVACGLLRTPRPKPAGASSPAPRTALQPQESVGAGAGEAALPLPGLL

SEQ ID NO: 129 - protein, 758 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSRGPRSLRGRDAPAPTPCVPAECFDLLVRHCVACGLLRTPRPKPAGASSPAPRTALQPQESVGAGAGEAALPLPGLLGGGGSRGPRSLRGRDAPAPTPCVPAECFDLLVRHCVACGLLRTPRPKPAGASSPAPRTALQPQESVGAGAGEAALPLPGLL

SEQ ID NO: 130 - protein, 758 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKG

SEQ ID NO: 131 - protein, 607 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESGGGGSGGGGSRGPRSLRGRDAPAPTPCVPAECFDLLVRHCVACGLLRTPRPKPAGASSPAPRTALQPQES

SEQ ID NO: 132 - protein, 530 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESGGGGSRGPRSLRGRDAPAPTPCVPAECFDLLVRHCVACGLLRTPRPKPAGASSPAPRTALQPQES

SEQ ID NO: 133 - protein, 502 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEFVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 134 - protein, 689 aa, synthetic construct; identical to SEQ ID NO: 79

SEQ ID NO: 135 - protein, 573 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 136 - protein, 434 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 137 - protein, 519 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 138 - protein, 606 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 139 - protein, 543 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 140 - protein, 493 aa, synthetic construct
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPGASSPAPRTALQPQESVGAGAGEAALPLPGLL

SEQ ID NO: 141 - protein, 536 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 142 - protein, 428 aa, synthetic construct
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 143 - protein, 953 aa, synthetic construct
KNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKGGGGSGGGGSGGGGSGGGGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPGASSPAPRTALQPQESVGAGAGEAALPLPGLL

SEQ ID NO: 144 - protein, 748 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSERQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 145 - protein, 769 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 146 - protein, 496 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSGGGSLQMAGQCSQNEYFDSLLHACIPCQLRCSSNTPPLTCQRYCNASVTNSVKGTNA

SEQ ID NO: 147 - protein, 411 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 148 - protein, 473 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNAGGGGSGGGSLQMAGQCSQNEYFDSLLHACIPCQLRCSSNTPPLTCQRYCNASVTNSVKGTNA

SEQ ID NO: 149 - protein, 535 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNAGGGGSGGGSLQMAGQCSQNEYFDSLLHACIPCQLRCSSNTPPLTCQRYCNASVTNSVKGTNAGGGGSGGGSLQMAGQCSQNEYFDSLLHACIPCQLRCSSNTPPLTCQRYCNASVTNSVKGTNA

SEQ ID NO: 150 - protein, 605 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSGGGSLQMAGQCSQNEYFDSLLHACIPCQLRCSSNTPPLTCQRYCNASVTNSVKGTNA

SEQ ID NO: 151 - protein, 471 aa, synthetic construct
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 152 - protein, 511 aa, synthetic construct
EMGTADLGPSSVPTPTNVTIESYNMNPIVYWEYQIMPQVPVTVEVKNYGVKNSEWIDCINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 153 - protein, 490 aa, synthetic construct
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNAGGGGSGGGSRGPRSLRGRDAPAPTPCVPAECFDLLVRHCVACGLLRTPRPKPAGASSPAPRTALQPQESVGAGAGEAALPLPGLL

SEQ ID NO: 154 - protein, 525 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 155 - protein, 775 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSERQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 156 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 157 - protein, 596 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 158 - protein, 592 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPDVFLFPPKPKDTLMISRTPEVTDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 159 - protein, 610 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSAAARGPRSLRGRDAPAPTPCVPAECFDLLVRHCVACGLLRTPRPKPAGASSPAPRTALQPQESVGAGAGEAALPLPGLL

SEQ ID NO: 160 - protein, 596 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPSVFLFPPKPKDTLMISRTPEVTCVVVDVSHEDPEVKFNWYVDGVEVHAAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 161 - protein, 596 aa, synthetic construct; identical to SEQ ID NO: 160

SEQ ID NO: 162 - protein, 596 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 163 - protein, 601 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWEPKSSDKTHTCPSCSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLL

SEQ ID NO: 164 - protein, 596 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSRGPRSLRGRDAPAPTPCVPAECFDLLVRHCVACGLLRTPRPKPAGASSPAPRTALQPQESVGAGAGEAALPLPGLL

SEQ ID NO: 165 - protein, 684 aa, synthetic construct
LQMAGQCSQNEYFDSLLHACIPCQLRCSSNTPPLTCQRYCNASVTNSVKGTNAGGGGSLVPRGSGGGGLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNADHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 166 - protein, 659 aa, synthetic construct
AQQCFHSEYFDSLLHACKPCHLRCSNPPATCQPYCDPSVTSSVKGTYTGGGGSLVPRGSGGGGAQQCFHSEYFDSLLHACKPCHLRCSNPPATCQPYCDPSVTSSVKGTYTGGGGSLVPRGSGGGGAQQCFHSEYFDSLLHACKPCHLRCSNPPATCQPYCDPSVTSSVKGTYTDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 167 - protein, 486 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 168 - protein, 572 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 169 - protein, 658 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 170 - protein, 557 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLLAEAAAKEAAAKEAAAKELQMAGQCSQNEYFDSLLHACIPCQLRCSSNTPPLTCQRYCNASVTNSVKGTNA

SEQ ID NO: 171 - protein, 471 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKELQMAGQCSQNEYFDSLLHACIPCQLRCSSNTPPLTCQRYCNASVTNSVKGTNA

SEQ ID NO: 172 - protein, 534 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLAEAAAKEAAAKEAAAKELQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNA

SEQ ID NO: 173 - protein, 589 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNA

SEQ ID NO: 174 - protein, 539 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSRGPRSLRGRDAPAPTPCVPAECFDLLVRHCVACGLLRTPRPKPAGASSPAPRTALQPQESVGAGAGEAALPLPGLLGGGGSLQMAGQCSQNEYFDSLLHACIPCQLRCSSNTPPLTCQRYCNASVTNSVKGTNA

SEQ ID NO: 175 - protein, 458 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNA

SEQ ID NO: 176 - protein, 516 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNAGGGGSLQMAGQCSQNEYFDSLLHACIPCQLRCSSNTPPLTCQRYCNASVTNSVKGTNA

SEQ ID NO: 177 - protein, 574 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLQMAGQCSQNEYFDSLLHACIPCQLRCSSNTPPLTCQRYCNASVTNSVKGTNAGGGGSLQMAGQCSQNEYFDSLLHACIPCQLRCSSNTPPLTCQRYCNASVTNSVKGTNAGGGGSLQMAGQCSQNEYFDSLLHACIPCQLRCSSNTPPLTCQRYCNASVTNSVKGTNA

SEQ ID NO: 178 - protein, 773 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEA

SEQ ID NO: 179 - protein, 1278 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLAEAAAKEAAAKEAAAKEAAAKAAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 180 - protein, 636 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVDKTHTCPPCPAPEAAGGPSVFLFPPKPKDTNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 181 - protein, 501 aa, synthetic construct
HNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYIDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSRGPRSLRGRDAPAPTPCVPAECFDLLVRHCVACGLLRTPRPKPAGASSPAPRTALQPQESVGAGAGEAALPLPG

SEQ ID NO: 182 - protein, 600 aa, synthetic construct
HNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYIDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 183 - protein, 629 aa, synthetic construct
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 184 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 646 aa, synthetic construct
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNAGGGGSGGGGSLQMAGQCSQNEYFDSLLHACIPCQLRCSSNTPPLTCQRYCNASVTNSVKGTNA

SEQ ID NO: 186 - protein, 215 aa, synthetic construct; identical to SEQ ID NO: 184

SEQ ID NO: 187 - protein, 715 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSGGGGSGGGGSGGGSRGPRSLRGRDAPAPTPCVPAECFDLLVRHCVACGLLRTPRPKPAGASSPAPRTALQPQESVGAGAGEAALPLPGLL

SEQ ID NO: 188 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 724 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 190 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 631 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 192 - protein, 634 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 193 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 811 aa, synthetic construct
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 196 - protein, 215 aa, synthetic construct
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 619 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGGGGGSGGGGSRGPRSLRGRDAPAPTPCVPAECFDLLVRHCVACGLLRTPRPKPAGASSPAPRTALQPQESVGAGAGEAALPLPG

SEQ ID NO: 198 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 623 aa, synthetic construct
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTYLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 200 - protein, 214 aa, synthetic construct
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 628 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 202 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 624 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 204 - protein, 213 aa, synthetic construct
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVAGVETTTPSKQSNNKYAASSYLSLTPEQWKSHRSYSCQVTHEGSTVEKTVAPTECS

SEQ ID NO: 205 - protein, 535 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPG

SEQ ID NO: 206 - protein, 213 aa, synthetic construct; identical to SEQ ID NO: 198

SEQ ID NO: 207 - protein, 642 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGGGGGSGGGGSRGPRSLRGRDAPAPTPCVPAECFDLLVRHCVACGLLRTPRPKPAGASSPAPRTALQPQESVGAGAGEAALPLPGGGGGSGGGGSRGPRSLRGRDAPAPTPCVPAECFDLLVRHCVACGLLRTPRPKPAGASSPAPRTALQPQESVGAGAGEAALPLPG

SEQ ID NO: 208 - protein, 213 aa, synthetic construct; identical to SEQ ID NO: 198

SEQ ID NO: 209 - protein, 564 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 210 - protein, 213 aa, synthetic construct; identical to SEQ ID NO: 198

SEQ ID NO: 211 - protein, 738 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 212 - protein, 213 aa, synthetic construct; identical to SEQ ID NO: 198

SEQ ID NO: 213 - protein, 912 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 214 - protein, 213 aa, synthetic construct; identical to SEQ ID NO: 198

SEQ ID NO: 215 - protein, 454 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSLQMAGQCSQNEYFDSLLHACIPCQLRCSSNTPPLTCQRYCNASVTNSVKGTNA

SEQ ID NO: 216 - protein, 213 aa, synthetic construct; identical to SEQ ID NO: 198

SEQ ID NO: 217 - protein, 518 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSLQMAGQCSQNEYFDSLLHACIPCQLRCSSNTPPLTCQRYCNASVTNSVKGTNAGGGGSGGGGSSLQMAGQCSQNEYFDSLLHACIPCQLRCSSNTPPLTCQRYCNASVTNSVKGTNA

SEQ ID NO: 218 - protein, 213 aa, synthetic construct; identical to SEQ ID NO: 198

SEQ ID NO: 219 - protein, 582 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSLQMAGQCSQNEYFDSLLHACIPCQLRCSSNTPPLTCQRYCNASVTNSVKGTNAGGGGSGGGGSSLQMAGQCSQNEYFDSLLHACIPCQLRCSSNTPPLTCQRYCNASVTNSVKGTNAGGGGSGGGGSSLQMAGQCSQNEYFDSLLHACIPCQLRCSSNTPPLTCQRYCNASVTNSVKGTNA

SEQ ID NO: 220 - protein, 213 aa, synthetic construct; identical to SEQ ID NO: 198

SEQ ID NO: 221 - protein, 650 aa, synthetic construct
QKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 222 - protein, 213 aa, synthetic construct; identical to SEQ ID NO: 198

SEQ ID NO: 223 - protein, 538 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 224 - protein, 213 aa, synthetic construct; identical to SEQ ID NO: 204

SEQ ID NO: 225 - protein, 626 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 226 - protein, 213 aa, synthetic construct; identical to SEQ ID NO: 204

SEQ ID NO: 227 - protein, 515 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQMAGQCSQNEYFDSLLHACIPCQLRCSSNTPPLTCQRYCNASVTNSVKGTNA

SEQ ID NO: 228 - protein, 213 aa, synthetic construct; identical to SEQ ID NO: 204

SEQ ID NO: 229 - protein, 717 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 230 - protein, 213 aa, synthetic construct; identical to SEQ ID NO: 204

SEQ ID NO: 231 - protein, 978 aa, synthetic construct
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSGGGGSGGGGSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKR

SEQ ID NO: 232 - protein, 215 aa, synthetic construct; identical to SEQ ID NO: 184

SEQ ID NO: 233 - protein, 623 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 234 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 624 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 236 - protein, 219 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSRGPRSLRGRDAPAPTPCVPAECFDLLVRHCVACGLLRTPRPKPAGASSPAPRTALQPQESVGAGAGEAALPLPGLL

SEQ ID NO: 238 - protein, 522 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 239 - protein, 630 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 240 - protein, 688 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGSSSSLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLW

SEQ ID NO: 241 - protein, 562 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGSSSSRGPRSLRGRDAPAPTPCVPAECFDLLVRHCVACGLLRTPRPKPAGASSPAPRTALQPQESVGAGAGEAALPLPGLLGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 242 - protein, 528 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 243 - protein, 420 aa, synthetic construct
LRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGGGGSGGGGSGGGGSGVSDFYKKLIEKAKTVEGVEALKDAIGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 244 - protein, 381 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRGVSDFYKKLIEKAKTVEGVEALKDAIGGGGSGGGGSGGGGSRGPRSLRGRDAPAPTPCVPAECFDLLVRHCVACGLLRTPRPKPAGASSPAPRTALQPQESVGAGAGEAALPLPGLLGGGGSGGGGSGGGGSRGPRSLRGRDAPAPTPCVPAECFDLLVRHCVACGLLRTPRPKPAGASSPAPRTALQPQESVGAGAGEAALPLPGLL

SEQ ID NO: 245 - protein, 581 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 246 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 247 - protein, 585 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 248 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 249 - protein, 591 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 250 - protein, 214 aa, synthetic construct; identical to SEQ ID NO: 246

SEQ ID NO: 251 - protein, 595 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGKQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 252 - protein, 215 aa, synthetic construct; identical to SEQ ID NO: 248

(19) United States
(12) Patent Application Publication
(10) Pub. No.: US 2025/0197475 A1
(43) Pub. Date: Jun. 19, 2025

(54) COMPOSITIONS AND METHODS FOR TREATMENT OF SJÖGREN'S SYNDROME AND/OR SYSTEMIC LUPUS ERYTHEMATOSUS

(71) Applicant: FAB BIOPHARMA, INC., Elizabeth, WV (US)

(72) Inventors: Gardiner SMITH, Elizabeth, WV (US); Chia Chia SUN, Elizabeth, WV (US); Reiner GENTZ, Elizabeth, WV (US); William FREIMUTH, Elizabeth, WV (US)

(21) Appl. No.: 18/847,801

(22) PCT Filed: Mar. 17, 2023
(86) PCT No.: PCT/US2023/064627

§ 371 Date: Sep. 17, 2024

Related U.S. Application Data
(60) Provisional application No. 63/321,179, filed on Mar. 18, 2022.

(51) Int. Cl. C07K 14/715; C07K 14/705

(52) U.S. Cl. CPC C07K 14/7155; C07K 14/70532; C07K 14/70578; C07K 14/7156; C07K 2319/30

(57) ABSTRACT

Fusion polypeptides are provided and comprise at least two ligand binding domains and a fragment crystallizable (Fc) region of immunoglobulin G (IgG). The ligand binding domains include an amino acid sequence selected from the group consisting of SEQ ID NOS: 1-25 and/or one of the at least two ligand binding domains binds modulates B cell activity while the other modulates T cell activity. Isolated nucleic acids, vectors, and isolated cells encoding or including the fusion peptides are further provided. Pharmaceutical compositions include the fusion peptides and a pharmaceutically-acceptable vehicle, carrier, or excipient. Methods of treating Sjögren's Syndrome and/or Systemic Lupus Erythematosus are also provided and comprise administering to a subject in need thereof the fusion polypeptide including the two ligand binding domains and the Fc region of IgG.

### RELATED APPLICATIONS

This application claims priority from U.S. Provisional Application Ser. No. 63/321,179, filed Mar. 18, 2022, the entire disclosure of which is incorporated herein by this reference.

### TECHNICAL FIELD

The presently-disclosed subject matter generally relates to compositions and methods for treatment of diseases predominantly occurring in women, such as Sjögren's Syndrome and/or Systemic Lupus Erythematosus, which include both T cell and B cell activity and related pathology. In particular, certain embodiments of the presently-disclosed subject matter relate to fusion proteins including multiple ligand binding domains for targeting and/or binding of one or more cytokines, growth factors, hormones, enzymes, or cell surface receptors associated with Sjögren's Syndrome and/or Systemic Lupus Erythematosus as well as their underlying pathologies.

### BACKGROUND

Sjögren's Syndrome, or Sjögren's disease, which may also be referred to herein simply as “SS,” is an autoimmune disorder that is often overlooked as its symptoms are frequently classified as being “mild” in nature. However, Sjögren's Syndrome is a debilitating disease with approximately 200,000 incident cases a year and affects up to 3 million people in the U.S. alone, with women being affected more than men (in a 9:1 ratio). Sjögren's Syndrome is primarily characterized by chronic inflammation of an individual's exocrine glands. That chronic inflammation, in turn, leads to a reduced ability of the affected exocrine glands to perform their secretory functions, with the “dry” glands being most often observed in the exocrine glands of the mouth and eyes as well as in the exocrine glands associated with the vaginal, oral, and gastrointestinal membranes. Due to the dryness, and accompanying inflammation and pain, Sjögren's Syndrome may lead to manifestations such as a loss of teeth or the development of other conditions, such as lymphoma (e.g., B cell). Moreover, it has been observed that Sjögren's Syndrome shares underlying immune-related disease mechanisms with a number of serious pathologies, including rheumatoid arthritis, thyroid dysregulation, Systemic Lupus Erythematosus, cervical cancer, and/or myasthenia gravis.

Sjögren's Syndrome is associated with over-expression and alteration of B white blood cells which are often accompanied by abnormally high levels of the B-cell activating factor (BAFF) cytokine. BAFF has been observed to be a significant molecule that is involved in not only the activation and stimulation of B-cell lymphocytes, but is often regarded as a leading factor in Sjögren's Syndrome pathogenesis. Additionally, it has been observed that a general dysregulation in cytokine regulation occurs in Sjögren's Syndrome and, more specifically, it has been observed that in individuals affected with Sjögren's Syndrome proinflammatory cytokines, such as Interferon (IFN) α and γ, tumor necrosis factor (TNF) α, interleukin (IL)-12 and IL-18 as well as other cytokines important in T and B cell activation and autoantibody production, such as IL-6 and BAFF, are often overexpressed. A variant of BAFF, designated BAFFvar, has also been cited as being associated with patients having an increased progression of autoimmune disease. The BAFFvar mutation increases the amount of BAFF as the half-life of its mRNA is increased. There is additionally a promoter mutation producing more BAFF Receptor (BAFFR) which is associated with non-Hodgkin lymphoma. This mutation results in higher BAFF activity. In this regard, it is believed to be possible to perform clinical trials with patients that have higher (altered) levels of BAFF and have, in addition, increased IFN-α or IFN-γ levels or of other cytokines of interest. BAFFvar may also be used in the evaluation of patients for clinical trials in combination with other biomarkers or baseline predictors associated with Sjogren's, Lupus and other autoimmune diseases.

Along these lines, and as indicated above, Systemic Lupus Erythematosus or “SLE” is often observed to accompany SjOgren's Syndrome and is similarly characterized by an immune imbalance associated with the abnormal production and activity of several cytokines, including, but not limited to, IFN-α, IFN-γ, BAFF, APRIL, IL-2, IL-12, IL-18, IL-21, IL-23, IL-10, IL-33, TNFα, CD40, IL-17, and IL-6. The dysregulation of these cytokines then leads to a loss of tolerance in individuals affected with SLE as well as a sustained autoantibody production that manifests itself as an abnormal activation of T cells and B cells. The abnormal activation of those cells then, in turn, leads to the formation of antigen-antibody complexes in tissues and organs and thereby results in inflammation and eventual damage to those tissues and organs.

Despite the recognized role played by cytokines in Sjögren's Syndrome and SLE, effective therapy for the conditions has, to date, remained elusive with the primary modes of treatment being focused on the symptoms that are manifested rather than the underlying cause of those symptoms. Indeed, treatment for Sjögren's Syndrome has largely focused on management of the accompanying dry eye and dry mouth through the use of over-the-counter eye drops and by sipping water more frequently. Likewise, conventional treatment for SLE has focused on the use of glucocorticoids and immunosuppressants, but those treatments often have poor specificity and result in the development of tolerance in the affected individuals.

Accordingly, additional compositions and methods capable of treating Sjögren's Syndrome and/or SLE including, more particularly, the T-cell, B-cell, and cytokine dysregulation observed in those conditions, would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes compositions and methods for treatment of diseases predominantly occurring in women, such as Sjögren's Syndrome and/or Systemic Lupus Erythematosus, which include both T cell and B cell activity and related pathology. In particular, certain embodiments of the presently-disclosed subject matter include fusion proteins including multiple ligand binding domains for targeting and/or binding of one or more cytokines, growth factors, hormones, enzymes, or cell surface receptors associated with Sjögren's Syndrome and/or Systemic Lupus Erythematosus as well as their underlying pathologies.

In some embodiments of the presently-disclosed subject matter, a fusion polypeptide is provided that comprises at least two ligand binding domains, with each ligand binding domain having an amino acid sequence selected from the group consisting of SEQ ID NOS: 1-25 or a fragment or variant thereof. The fusion protein further comprises a fragment crystallizable (Fc) region of immunoglobulin G (IgG) or a fragment or variant thereof. In some embodiments, the fusion polypeptide comprises two ligand binding domains, while, in other embodiments, the fusion polypeptide comprises three ligand binding domains. In some embodiments, at least two ligand binding domains are included and are different and, in certain embodiments where the fusion polypeptide comprises at least three ligand binding domains, at least two of the ligand binding domains are the same.

In some embodiments of the presently-described fusion polypeptides, the Fc region included in the fusion polypeptide comprises an amino acid sequence selected from the group consisting of SEQ ID NOS: 26-42. In some embodiments, a linker peptide is further included in the fusion polypeptides for connecting the at least two ligand binding domains to each other and/or to the Fc region. In some embodiments, the linker polypeptide has a sequence selected from the group consisting of SEQ ID NOS: 43-71.

With further regard to the particular fusion polypeptides provided by the presently-disclosed subject matter, in some embodiments, the fusion polypeptide comprises a sequence selected from the group consisting of SEQ ID NOS: 72-182, such as, in certain embodiments, a fusion polypeptide comprising a sequence selected from the group consisting of SEQ ID NOS: 102-119, a fusion polypeptide comprising a sequence selected from the group consisting of SEQ ID NOS: 73-74 and 118-119, a fusion polypeptide comprises the sequence of SEQ ID NO: 120, or a fusion polypeptide comprising a sequence selected from the group consisting of SEQ ID NOS: 72-124. In some embodiments, the fusion polypeptide comprises the sequence of SEQ ID NO: 121 or 122. In some embodiments of the presently-described fusion polypeptide, the fusion polypeptide comprises a sequence selected from the group consisting of SEQ ID NOS: 183-252.

In some embodiments of the fusion polypeptides, the ligand binding domain included in the fusion polypeptide comprises a BAFFR selected from the group consisting of SEQ ID NOS: 2-8. In some embodiments, the fusion protein comprises two or more BAFFR selected from the group consisting of SEQ ID NOS: 2-8. In other embodiments, the ligand binding domain comprises an IL-17 Receptor having the sequence of SEQ ID NO: 1. In still other embodiments, the ligand binding domain comprises an IL-21 Receptor selected from the group consisting of SEQ ID NO: 16 and 24. In some embodiments, the ligand binding domain comprises a TNF Receptor selected from the group consisting of SEQ ID NOS: 10-12 and 19, and, in some embodiments, the ligand binding domain comprises a IFN Receptor selected from the group consisting of SEQ ID NOS: 9 and 14-15.

In some embodiments, a fusion polypeptide is provided that comprises an Fc region having a sequence selected from the group consisting of SEQ ID NOS: 26-42. In some such embodiments, the Fc region has a sequence selected from: the group consisting of SEQ ID NOS: 29 or 30; the sequence of SEQ ID NO: 37; or the group consisting of SEQ ID NOS: 26-41.

In some embodiment of the fusion polypeptides, the at least two ligand binding domains comprises at least one ligand binding domain that reduces B cell activity and at least one ligand binding domain that reduces T cell activity. In some embodiments, the at least one ligand binding domain that reduces B cell activity and/or the at least one ligand binding domain that reduces T cell activity slows the progression of Sjogren's Syndrome.

Further provided by the presently-disclosed subject matter are isolated nucleic acids, vectors, and isolated cells encoding or otherwise including a fusion polypeptide of the presently-disclosed subject matter. In some embodiments, an isolated nucleic acid sequence encoding a fusion polypeptide is provided where the fusion polypeptide includes at least two ligand binding domains, with each ligand binding domain having an amino acid sequence selected from the group consisting of SEQ ID NOS: 1-25 or a fragment or variant thereof. The nucleic acid further encodes a Fc region from IgG, or a fragment or variant thereof, that is included in the fusion polypeptide. In some embodiments, the nucleic acid encodes a fusion polypeptide having a sequence selected from the group consisting of SEQ ID NOS: 72-182.

With regard to the vectors provided by the presently-disclosed subject matter, in some embodiments, a vector is provided comprising the above-described isolated nucleic acid sequences. In some embodiments of the vectors, the isolated nucleic acid is operatively linked to an expression cassette.

With regard to the isolated cells provided by the presently-disclosed subject matter, in some embodiments, an isolated cell is provided that comprises a nucleic acid sequence encoding a fusion polypeptide including: at least two ligand binding domains, with each ligand binding domain having an amino acid sequence selected from the group consisting of SEQ ID NOS: 1-25 or a fragment or variant thereof, and a Fc region of IgG or a fragment or variant thereof.

Still further provided by the presently-disclosed subject matter are pharmaceutical compositions. In some embodiments, a pharmaceutical composition is provided that comprises a fusion polypeptide including: at least two ligand binding domains, with each ligand binding domain having an amino acid sequence selected from the group consisting of SEQ ID NOS:1-25 or a fragment or variant thereof, a Fc region of IgG or a fragment or variant thereof, and a pharmaceutically-acceptable vehicle, carrier, or excipient.

Even further provided by the presently-disclosed subject matter are methods for treating Sjögren's Syndrome and/or Systemic Lupus Erythematosus. In some embodiments, a method for treating Sjögren's Syndrome and/or Systemic Lupus Erythematosus comprises administering to a subject in need thereof a fusion polypeptide of the presently-disclosed subject matter, such as one that includes at least two ligand binding domains, where each ligand binding domain having an amino acid sequence selected from the group consisting of SEQ ID NOS: 1-25 or a fragment or variant thereof, and a Fc region of IgG or a fragment or variant thereof.

In some embodiments of the fusion polypeptides described herein, the fusion polypeptides are comprised of a monoclonal antibody binding domain and a ligand binding domain. For instance, in some embodiments, a fusion polypeptide is provided that comprises a monoclonal antibody binding domain for binding CD20 and a ligand binding domain from PD1. In some embodiments, an exemplary fusion polypeptide comprises a monoclonal antibody binding domain for binding CD20 and one, two, or three ligand binding domains for binding BAFF. In other embodiments, a fusion polypeptide is provided that comprises a monoclonal antibody binding domain for binding CD20 and one, two, or three ligand binding domains from TACI. In yet other embodiments, a fusion polypeptide is provided that comprises a monoclonal antibody binding domain for binding CD20, one, two, or three ligand binding domains for binding BAFF, and a ligand binding domain from BCMA. In some embodiments, a fusion polypeptide is provided that comprises a monoclonal antibody binding domain for binding BAFF, a ligand binding domain for binding BAFF, and a ligand binding domain from TACI. In some embodiments, a fusion polypeptide is provided that comprises a monoclonal antibody binding domain for binding BAFF and a ligand binding domain from BCMA.

With further regard to the fusion polypeptides comprising a monoclonal antibody binding domain, in some such embodiments, a fusion polypeptide is provided that comprises the sequences of SEQ ID NOS: 203-204; 223-224; 225-226; 227-228; 229-230; 231-232; 233-234; 235-236.

In other embodiments of the presently-disclosed subject matter, further fusion peptides are provided that, in certain embodiments, comprise the sequence of SEQ ID NO: 237; 238; 239; 240; 241; or 242. In some embodiments, a fusion protein is provided that further comprises an albumin binding site, such as, in certain embodiments, a fusion polypeptide comprising the sequence of SEQ ID NO: 243 or 244. In some embodiments, a fusion polypeptide is provided that comprises one or more binding domains from a bispecific antibody and one or more ligand binding domains for a cytokine. In some such embodiments, the fusion polypeptide comprises the sequences of SEQ ID NOS: 245-248 or the sequences of SEQ ID NOS: 249-252.

Still even further provided by the presently-disclosed subject matter are fusion polypeptides that comprise: at least two ligand binding domains, where one of the at least two ligand binding domains binds modulates B cell activity and the other of the at least two ligand binding domains modulates T cell activity; and a Fc region of IgG or a fragment or variant thereof. Such fusion polypeptides, in some embodiments, are used in a method of treating a subject having a mutation in an amino acid sequence or nucleic acid sequence encoding BAFF, whereby an effective amount of the fusion polypeptide is administered to a subject. In some embodiments, the subject has a biomarker selected from: an antinuclear antibodies (ANA) titer greater than 1:80; and anti-dsDNA greater than 30 IU/ml′; an anti-Smith (Sm) antibody amount greater than 15 units/ml; a complement C3 amount less than 900 mg/liter; a complement C4 level less than 60 mg/liter; and a C-reactive protein (CRP) positive amount greater than 3 mg/ml. In some embodiments, the subject has or is at risk of developing Systemic Lupus Erythematosus flares.

Further features and advantages of the present invention will become evident to those of ordinary skill in the art after a study of the description, figures, and non-limiting examples in this document.

### BRIEF DESCRIPTION OF THE DRAWINGS

FIG. 1A includes schematic diagrams showing exemplary fusion protein constructs made in accordance with the presently-disclosed subject matter, and comprising one or more ligand binding domains (LBD) and a fragment crystallizable (Fc) region, and where each fusion protein is shown forming a homodimer with an identical fusion protein.

FIG. 1B includes schematic diagrams showing other exemplary fusion protein constructs made in accordance with the presently-disclosed subject matter, comprising one or more LBDs fused to a Fc region where the pairs of fusion proteins indicated in the figure are formed from two DNA constructs and where the Fc regions are engineered to form heterodimers.

FIG. 2A includes schematic diagrams showing exemplary fusion protein constructs made in accordance with the presently-disclosed subject matter, and comprising one or more ligand binding domains (LBD) fused to an antibody including a Fc region.

FIG. 2B includes schematic diagrams showing bi-specific antibody fusion proteins where the heavy chain (HC) of an antibody is fused to one or more LBDs, where the Fc region included in the HCs is engineered to form heterodimers, and where there are two light chains. 4 discrete genes can be included in a expression construct (e.g., a plasmid), for example, resulting in 3 binding sites.

FIG. 2C includes schematic diagrams showing bi-specific antibody fusion proteins where the heavy chain (HC) of an antibody is fused to two different LBDs, such that the fusion protein includes 4 binding sites.

FIG. 2D includes schematic diagrams showing bi-specific antibody fusion proteins where the heavy chain (HC) of an antibody includes different Fc regions and are fused to two similar LBDs, such that the fusion protein includes 3 binding sites.

FIG. 2E includes schematic diagrams showing in the alternative, a cytokine fused to a soluble receptor fusion protein, a cytokine fused to a Fc fusion protein with two ligand binding domains, two different cytokines fused to a Fc region, and a cytokine fused to the heavy chains of an antibody.

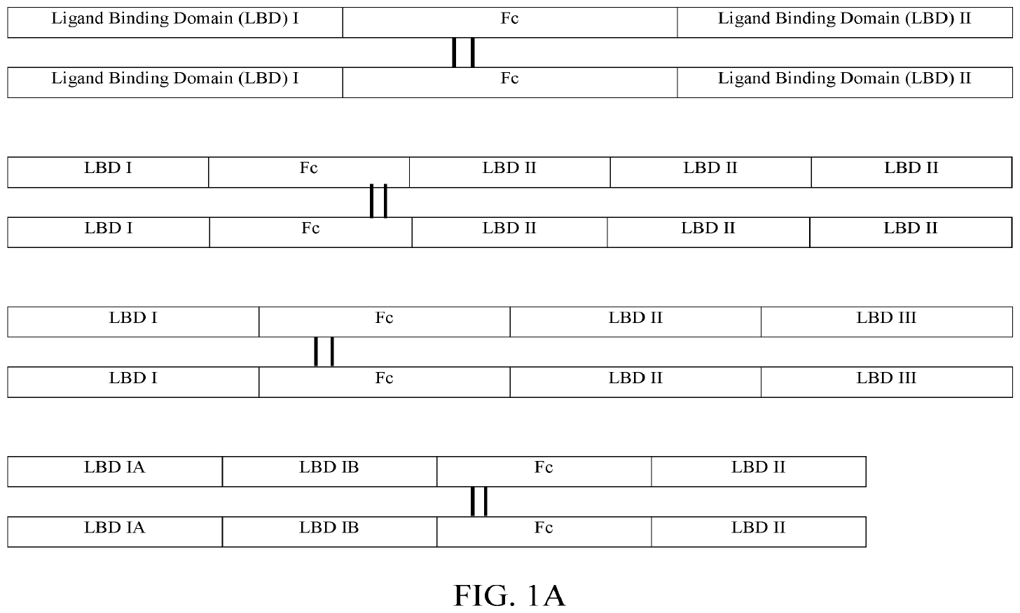

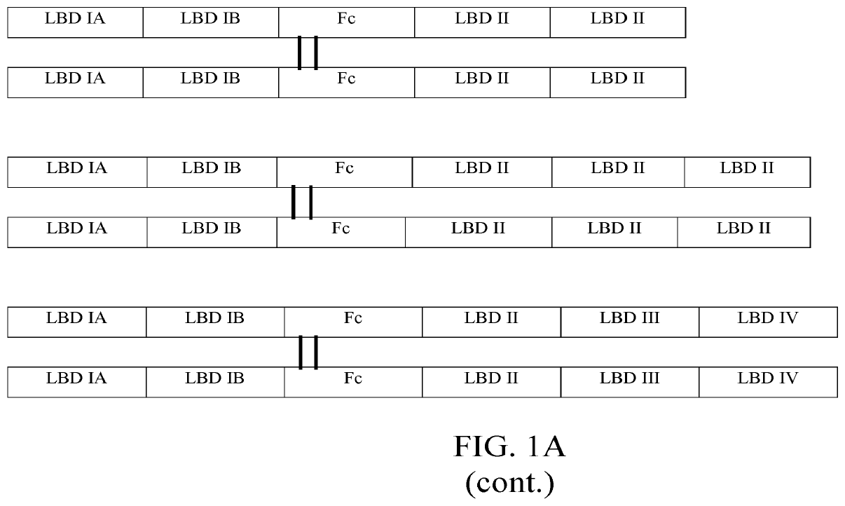

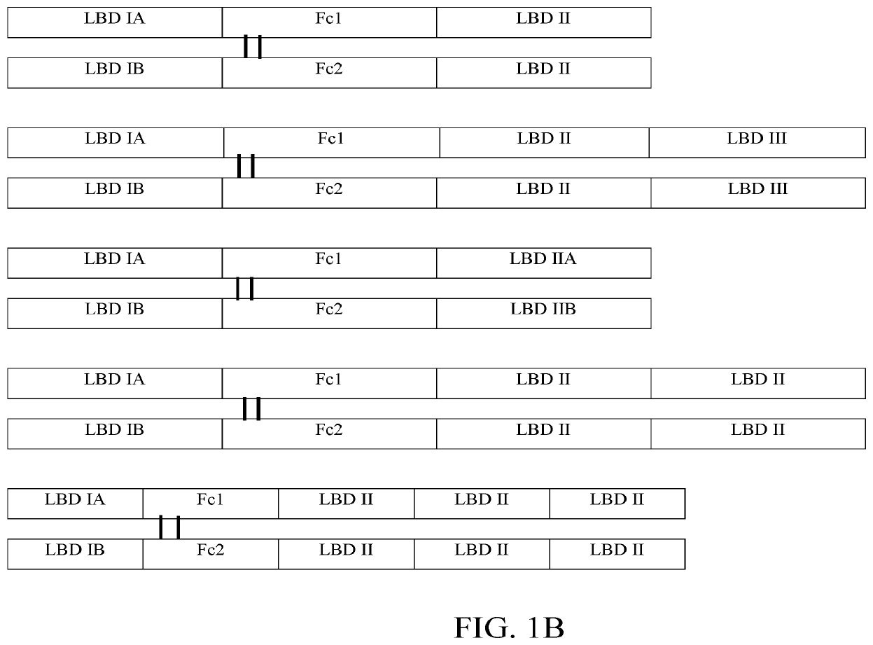

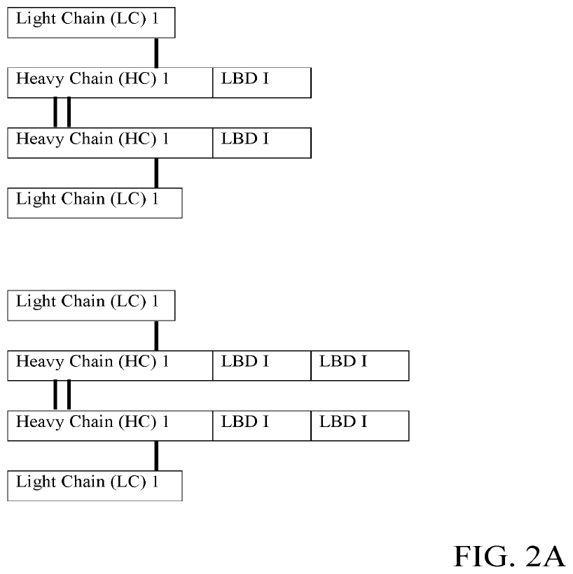

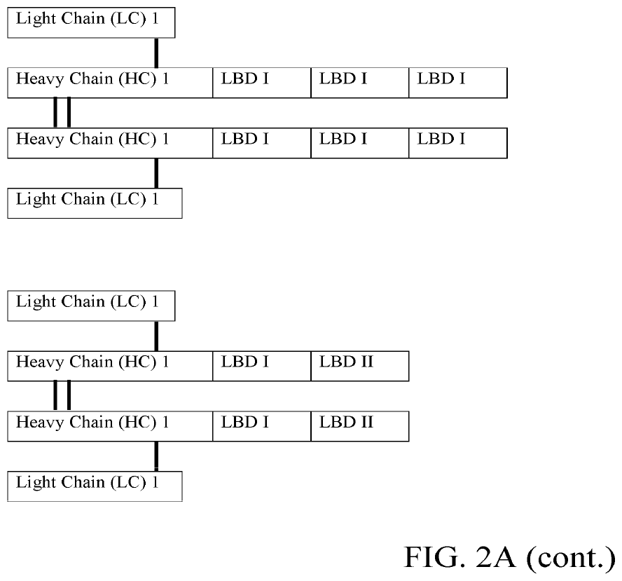

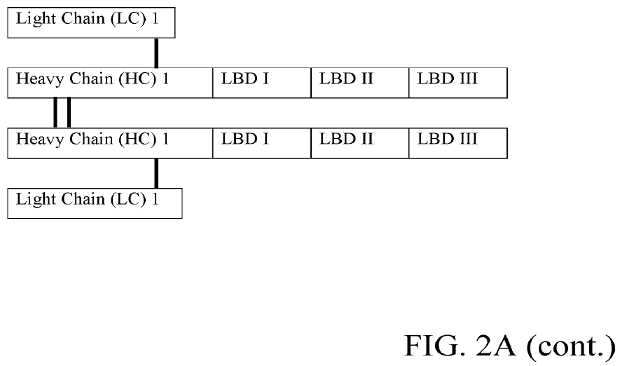

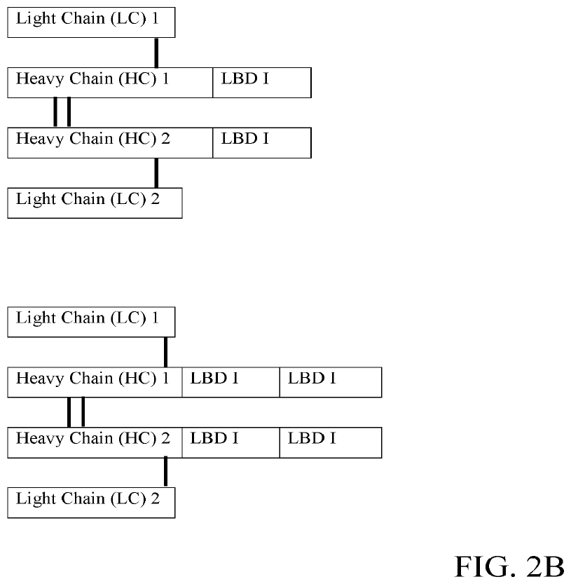

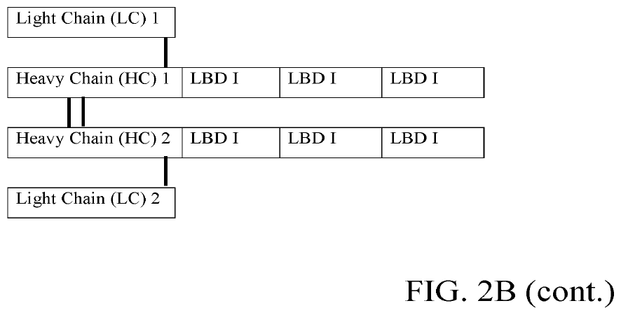

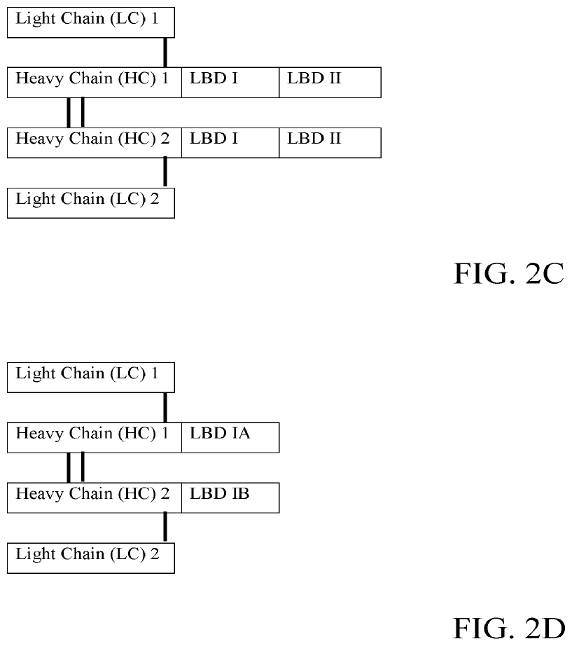

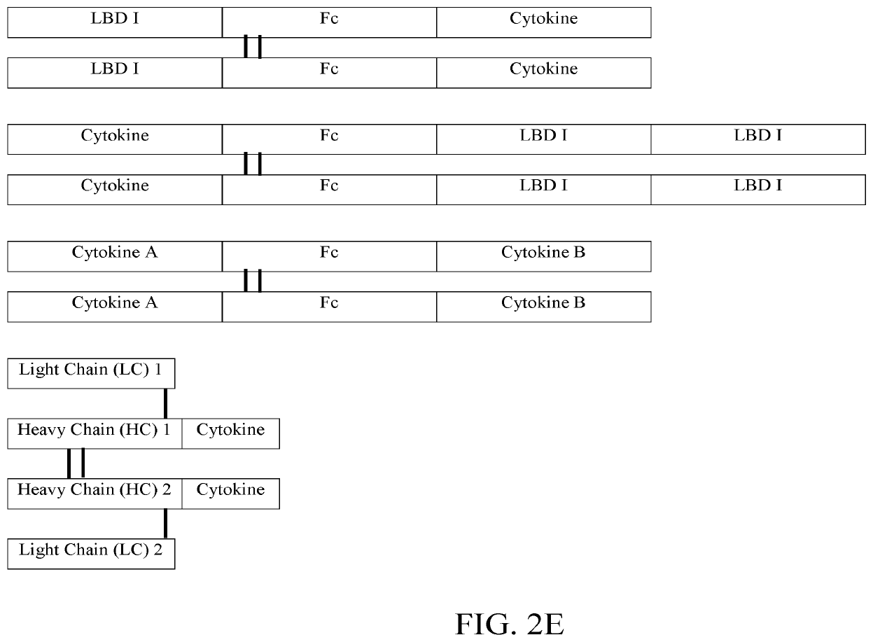

### BRIEF DESCRIPTION OF THE SEQUENCE LISTING

### Ligand Binding Sequences; Extracellular Domains

SEQ ID NO: 1 is an amino acid sequence of extracellular domain of an IL-17A receptor;
SEQ ID NO: 2 is an amino acid sequence of a binding domain of a B cell Activating Factor (BAFF) receptor (BAFFR);
SEQ ID NO: 3 is an amino acid sequence of a soluble BAFFR;
SEQ ID NO: 4 is an amino acid sequence of a soluble BAFFR;
SEQ ID NO: 5 is an amino acid sequence of a soluble BAFFR;
SEQ ID NO: 6 is an amino acid sequence of a soluble BAFFR;
SEQ ID NO: 7 is an amino acid sequence of a soluble BAFFR;
SEQ ID NO: 8 is an amino acid sequence of two BAFFR ligand binding domains with a flexible linker;
SEQ ID NO: 9 is an amino acid sequence of an extracellular binding domain of Interferon (IFN) Gamma Receptor 1 (IFNGR1);
SEQ ID NO: 10 is an amino acid sequence of an extracellular domain of Tumor Necrosis Factor Receptor (TNFR)-I;
SEQ ID NO: 11 is an amino acid sequence of an extracellular domain of TNFR-2;
SEQ ID NO: 12 is an amino acid sequence of an extracellular domain of TNFR-5;
SEQ ID NO: 13 is an amino acid sequence of an extracellular domain of a transmembrane activator and calcium modulator and cyclophilin ligand (CAML) interactor (TACI) receptor;
SEQ ID NO: 14 is an amino acid sequence of an extracellular domain of the Interferon Alpha Receptor (IFNAR) 1;
SEQ ID NO: 15 is an amino acid sequence of an extracellular domain of IFNAR2;
SEQ ID NO: 16 is an amino acid sequence of an extracellular domain of IL-21 Receptor;
SEQ ID NO: 17 is an amino acid sequence of an extracellular domain of Cytokine Receptor Common Gamma Chain (IL2RG);
SEQ ID NO: 18 is an amino acid sequence of a Cytotoxic T-Lymphocyte Protein 4 (CTLA-4);
SEQ ID NO: 19 is an amino acid sequence of a Tumor Necrosis Factor Receptor Superfamily Member 4 (CD134, OX40L Receptor);
SEQ ID NO: 20 is an amino acid sequence of an Inducible T-Cell Costimulatory Receptor (ICOS-R) (CD278);
SEQ ID NO: 21 is an amino acid sequence of a CD27 Antigen (Receptor);
SEQ ID NO: 22 is an amino acid sequence of a human IL-23 Receptor (IL23R; UniProtKB Q5VWK5);
SEQ ID NO: 23 is an amino acid sequence of a human IL-12 receptor subunit Beta 1 (IL-12RB1; UniProtKB P42701);
SEQ ID NO: 24 is an amino acid sequence of an IL-21 receptor (IL21R; UniProtKB Q9HBE5); and
SEQ ID NO: 25 is an amino acid sequence of a Tumor Necrosis Factor Receptor 9 Superfamily Member (TNFR9).

### Fc Sequences

SEQ ID NO: 26 is an amino acid sequence of a fragment crystallizable (Fc) Immunoglobulin G1 (IgG1) variant;
SEQ ID NO: 27 is an amino acid sequence of a Fc IgG1 variant;
SEQ ID NO: 28 is an amino acid sequence of a Fc IgG1 variant;
SEQ ID NO: 29 is an amino acid sequence of a Fc IgG1 variant;
SEQ ID NO: 30 is an amino acid sequence of a Fc IgG1 variant;
SEQ ID NO: 31 is an amino acid sequence of a Fc IgG1 variant;
SEQ ID NO: 32 is an amino acid sequence of a Fc IgG1 variant;
SEQ ID NO: 33 is an amino acid sequence of a Fc IgG1 variant;
SEQ ID NO: 34 is an amino acid sequence of a Fc IgG1 variant;
SEQ ID NO: 35 is an amino acid sequence of a Fc IgG1 variant;
SEQ ID NO: 36 is an amino acid sequence of a Fc IgG1 variant;
SEQ ID NO: 37 is an amino acid sequence of a Fc IgG4 variant;
SEQ ID NO: 38 is an amino acid sequence of a Fc IgG1 variant;
SEQ ID NO: 39 is an amino acid sequence of a Fc IgG2 variant;
SEQ ID NO: 40 is an amino acid sequence of a Fe IgG2 variant;
SEQ ID NO: 41 is an amino acid sequence of a Fc IgG4 variant; and
SEQ ID NO: 42 is an amino acid sequence of a Fc IgG1 variant.

### Linker Sequences

SEQ ID NO: 43 is an amino acid sequence of a flexible linker peptide;
SEQ ID NO: 44 is an amino acid sequence of an exemplary linker peptide for use between functional domains;
SEQ ID NO: 45 is another amino acid sequence of an exemplary linker peptide for use between functional domains;
SEQ ID NO: 46 is another amino acid sequence of an exemplary linker peptide for use between functional domains (helical);
SEQ ID NO: 47 is another amino acid sequence of an exemplary linker peptide for use between functional domains;
SEQ ID NO: 48 is another amino acid sequence of an exemplary linker peptide for use between functional domains;
SEQ ID NO: 49 is another amino acid sequence of an exemplary linker peptide for use between functional domains;
SEQ ID NO: 50 is another amino acid sequence of an exemplary linker peptide for use between functional domains;
SEQ ID NO: 51 is another amino acid sequence of an exemplary linker peptide for use between functional domains;
SEQ ID NO: 52 is another amino acid sequence of an exemplary linker peptide for use between functional domains;
SEQ ID NO: 53 is another amino acid sequence of an exemplary linker peptide for use between functional domains;
SEQ ID NO: 54 is another amino acid sequence of an exemplary linker peptide for use between functional domains;
SEQ ID NO: 55 is another amino acid sequence of an exemplary linker peptide for use between functional domains;
SEQ ID NO: 56 is another amino acid sequence of an exemplary linker peptide for use between functional domains;
SEQ ID NO: 57 is another amino acid sequence of an exemplary linker peptide for use between functional domains;
SEQ ID NO: 58 is another amino acid sequence of an exemplary linker peptide for use between functional domains;
SEQ ID NO: 59 is another amino acid sequence of an exemplary linker peptide for use between functional domains;
SEQ ID NO: 60 is another amino acid sequence of an exemplary linker peptide for use between functional domains;
SEQ ID NO: 61 is another amino acid sequence of an exemplary linker peptide for use between functional domains;
SEQ ID NO: 62 is another amino acid sequence of an exemplary linker peptide for use between functional domains;
SEQ ID NO: 63 is another amino acid sequence of an exemplary linker peptide for use between functional domains;
SEQ ID NO: 64 is another amino acid sequence of an exemplary linker peptide for use between functional domains;
SEQ ID NO: 65 is another amino acid sequence of an exemplary linker peptide for use between functional domains;
SEQ ID NO: 66 is another amino acid sequence of an exemplary linker peptide for use between functional domains;
SEQ ID NO: 67 is another amino acid sequence of an exemplary linker peptide for use between functional domains;
SEQ ID NO: 68 is another amino acid sequence of an exemplary linker peptide for use between functional domains;
SEQ ID NO: 69 is another amino acid sequence of an exemplary linker peptide for use between functional domains;
SEQ ID NO: 70 is another amino acid sequence of an exemplary linker peptide for use between functional domains; and
SEQ ID NO: 71 is another amino acid sequence of an exemplary linker peptide for use between functional domains.

### Fusion Protein Sequences (Including Ligand Binding/Extracellular Domains)

SEQ ID NO: 72 is an amino acid sequence of a fusion protein comprising IL-17 RA-Fc/IgG1-LINKER-BAFFR;
SEQ ID NO: 73 is an amino acid sequence of a fusion protein comprising IL-17 RA-Fc/IgG1-LINKER-BAFFR-LINKER-BAFFR;
SEQ ID NO: 74 is an amino acid sequence of a fusion protein comprising IL-17 RA-Fc/IgG1-LINKER-BAFFR-LINKER-BAFFR-LINKER-BAFFR;
SEQ ID NO: 75 is an amino acid sequence of a fusion protein comprising IL-17 AR-Fc-LINKER-IFN gamma RI;
SEQ ID NO: 76 is an amino acid sequence of a fusion protein comprising TNFR2-Fc-LINKER-BAFFR-LINKER-BAFFR;
SEQ ID NO: 77 is an amino acid sequence of a fusion protein comprising TNFR5-Fc-LINKER-BAFFR-LINKER-BAFFR;
SEQ ID NO: 78 is an amino acid sequence of a fusion protein comprising IFN gamma R-Fc-LINKER-BAFFR;
SEQ ID NO: 79 is an amino acid sequence of a fusion protein comprising IL-17 RA-Fc/IgG1-LINKER-TACI;
SEQ ID NO: 80 is an amino acid sequence of a fusion protein comprising IL-17 RA-Fc/IgG1-LINKER-BCMA;
SEQ ID NO: 81 is an amino acid sequence of a fusion protein comprising CTLA-4-Fc-LINKER-BAFFR;
SEQ ID NO: 82 is an amino acid sequence of a fusion protein comprising CTLA-4-Fc-LINKER-BAFFR-LINKER-BAFFR;
SEQ ID NO: 83 is an amino acid sequence of a fusion protein comprising CTLA-4-Fc-LINKER-BAFFR-LINKER-BAFFR-LINKER-BAFFR;
SEQ ID NO: 84 is an amino acid sequence of a fusion protein comprising TNFR2-Fc-LINKER-BAFFR;
SEQ ID NO: 85 is an amino acid sequence of a fusion protein comprising Ox40R-Fc-LINKER-BAFFR;
SEQ ID NO: 86 is an amino acid sequence of a fusion protein comprising IFNGR1-Fc-LINKER-BAFFR;
SEQ ID NO: 87 is an amino acid sequence of a fusion protein comprising ICOSR-Fc-LINKER-BAFFR;
SEQ ID NO: 88 is an amino acid sequence of a fusion protein comprising IL21 Receptor-linker-Common Gamma Chain-linker-Fc/IgG1-linker-BAFFR;
SEQ ID NO: 89 is an amino acid sequence of a fusion protein comprising IL21R-gammachain-linker-Fc-linker-BAFFR;
SEQ ID NO: 90 is an amino acid sequence of a fusion protein comprising IL-17 RA-Fc/IgG1-linker-BAFFR-linker-TACI;
SEQ ID NO: 91 is an amino acid sequence of a fusion protein comprising CTLA-4-Fc-linker-BAFFR-linker-BCMA;
SEQ ID NO: 92 is an amino acid sequence of a fusion protein comprising CTLA-4-Fc-linker-BCMA;
SEQ ID NO: 93 is an amino acid sequence of a fusion protein comprising CTLA-4-Fc-linker-BCMA-linker-BCMA;
SEQ ID NO: 94 is an amino acid sequence of a fusion protein comprising CTLA-4-Fc-LINKER-BCMA-LINKER-BCMA-LINKER-BCMA;
SEQ ID NO: 95 is an amino acid sequence of a fusion protein comprising CTLA-4-Fc/IgG4-LINKER-BAFFR-LINKER-BAFFR;
SEQ ID NO: 96 is an amino acid sequence of a fusion protein comprising TNFR2-Fc-linker-BAFFR-linker-BCMA;
SEQ ID NO: 97 is an amino acid sequence of a fusion protein comprising Ox40R-Fc-LINKER-BCMA;
SEQ ID NO: 98 is an amino acid sequence of a fusion protein comprising IFNGR1-Fc-LINKER-BCMA;
SEQ ID NO: 99 is an amino acid sequence of a fusion protein comprising ICOSR-Fc-linker-BCMA-linker-BAFFR;
SEQ ID NO: 100 is an amino acid sequence of a fusion protein comprising IFNAR2 (high affinity)-Fc-linker-BAFFR;
SEQ ID NO: 101 is an amino acid sequence of a fusion protein comprising IL21R-linker-gamma chain-Fc/IgG1-linker-BAFFR-linker-BAFFR;
SEQ ID NO: 102 is an amino acid sequence of a fusion protein comprising IL-17 RA-Fc/IgG1-linker-BAFFR;
SEQ ID NO: 103 is an amino acid sequence of a fusion protein comprising IL-17 RA-Fc/IgG1-linker-BAFFR;
SEQ ID NO: 104 is an amino acid sequence of a fusion protein comprising IL-17 RA-Fc/IgG1-linker-BAFFR;
SEQ ID NO: 105 is an amino acid sequence of a fusion protein comprising IL-17 RA-Fc/IgG1-linker-BAFFR;
SEQ ID NO: 106 is an amino acid sequence of a fusion protein comprising IL-17 RA-Fc/IgG1-linker-BAFFR;
SEQ ID NO: 107 is an amino acid sequence of a fusion protein comprising IL-17 RA-Fc/IgG1-BAFFR;
SEQ ID NO: 108 is an amino acid sequence of a fusion protein comprising IL-17 RA-Fc/IgG1-linker-BAFFR;
SEQ ID NO: 109 is an amino acid sequence of a fusion protein comprising IL-17 RA-Fc/IgG1-linker-BAFFR;
SEQ ID NO: 110 is an amino acid sequence of a fusion protein comprising IL-17 RA-Fc/IgG4-linker-BAFFR;
SEQ ID NO: 111 is an amino acid sequence of a fusion protein comprising IL-17 RA-Fc/IgG1-linker-BAFFR;
SEQ ID NO: 112 is an amino acid sequence of a fusion protein comprising IL-17 RA-Fc/IgG1-linker-BAFFR;
SEQ ID NO: 113 is an amino acid sequence of a fusion protein comprising IL-17 RA-Fc/IgG1-linker-BAFFR;
SEQ ID NO: 114 is an amino acid sequence of a fusion protein comprising IL-17 RA-Fc/IgG1-linker-BAFFR;
SEQ ID NO: 115 is an amino acid sequence of a fusion protein comprising IL-17 RA-Fc/IgG1-linker-BAFFR;
SEQ ID NO: 116 is an amino acid sequence of a fusion protein comprising IL-17 RA-Fc/IgG1-linker-BAFFR;
SEQ ID NO: 117 is an amino acid sequence of a fusion protein comprising IL-17 RA-Fc/IgG1-linker-BAFFR;
SEQ ID NO: 118 is an amino acid sequence of a fusion protein comprising IL-17 RA-Fc/IgG1-linker-BAFFR-linker-BAFFR;
SEQ ID NO: 119 is an amino acid sequence of a fusion protein comprising IL-17 RA-Fc/IgG1-linker-BAFFR-linker-BAFFR-linker-BAFFR;
SEQ ID NO: 120 is an amino acid sequence of a fusion protein comprising BAFFR-Fc/IgG1-linker-TACI;
SEQ ID NO: 121 is an amino acid sequence of a fusion protein comprising IL-21R-Fc(1)-linker-BAFFR;
SEQ ID NO: 122 is an amino acid sequence of a fusion protein comprising IL22RA-linker-IL10 beta-Fc-linker-BAFFR-linker-BAFFR;
SEQ ID NO: 123 is an amino acid sequence of a fusion protein comprising PD-1-Fc-linker-CTLA-4;
SEQ ID NO: 124 is an amino acid sequence of a fusion protein comprising LAG3-Fc-linker-CTLA-4;
SEQ ID NO: 125 is an amino acid sequence of a fusion protein comprising TIM-3 Extracellular Domain-Fc-linker-PD1;
SEQ ID NO: 126 is an amino acid sequence of LAG-3-Fc/IgG4-linker-PD1;
SEQ ID NO: 127 is an amino acid sequence of a fusion protein comprising IL-17 RA-Fc/IgG1-linker-BAFFR;
SEQ ID NO: 128 is an amino acid sequence of a fusion protein comprising IL-17 RA-Fc/IgG1-BAFFR-linker-BAFFR;
SEQ ID NO: 129 is an amino acid sequence of a fusion protein comprising IL-17 RA-Fc/IgG1-linker-BAFFR-linker-BAFFR-linker-BAFFR;
SEQ ID NO: 130 is an amino acid sequence of a fusion protein comprising IL-17 AR-Fc-linker-IFN gamma RI;
SEQ ID NO: 131 is an amino acid sequence of a fusion protein comprising TNFR2-Fc-linker-BAFFR-linker-BAFFR;
SEQ ID NO: 132 is an amino acid sequence of a fusion protein comprising TNFR5-Fc-linker-BAFFR-linker-BAFFR;
SEQ ID NO: 133 is an amino acid sequence of a fusion protein comprising IFN AR2-Fc-linker-BCMA;
SEQ ID NO: 134 is an amino acid sequence of a fusion protein comprising IL-17 RA-Fc/IgG1-linker-TACI;
SEQ ID NO: 135 is an amino acid sequence of a fusion protein comprising IL-17 RA-Fc/IgG1-linker-BCMA;
SEQ ID NO: 136 is an amino acid sequence of a fusion protein comprising CTLA-4-Fc-BAFFR;
SEQ ID NO: 137 is an amino acid sequence of a fusion protein comprising CD40-Fc-linker-BAFFR-linker-BAFFR;
SEQ ID NO: 138 is an amino acid sequence of a fusion protein comprising CD40-Fc-linker-BAFFR-linker-BAFFR-linker-BAFFR;
SEQ ID NO: 139 is an amino acid sequence of a fusion protein comprising TNFR2-Fc-linker-BAFFR;
SEQ ID NO: 140 is an amino acid sequence of a fusion protein comprising Ox40R-Fc-linker-BAFFR;
SEQ ID NO: 141 is an amino acid sequence of a fusion protein comprising IFNGR1-Fc-linker-BAFFR;
SEQ ID NO: 142 is an amino acid sequence of a fusion protein comprising ICOSR-Fc-linker-BAFFR;
SEQ ID NO: 143 is an amino acid sequence of a fusion protein comprising IFNAR1-linker-IFNAR2-Fc-linker-BAFFR;
SEQ ID NO: 144 is an amino acid sequence of a fusion protein comprising IL21R-linker-gamma chain-Fc-linker-BCMA;
SEQ ID NO: 145 is an amino acid sequence of a fusion protein comprising IL-17 RA-Fc/IgG1-linker-BAFFR-linker-TACI;
SEQ ID NO: 146 is an amino acid sequence of a fusion protein comprising CTLA-4-Fc-linker-BAFFR-linker-BCMA;
SEQ ID NO: 147 is an amino acid sequence of a fusion protein comprising CTLA-4-Fc-linker-BCMA;
SEQ ID NO: 148 is an amino acid sequence of a fusion protein comprising CTLA-4-Fc-linker-BCMA-linker-BCMA;
SEQ ID NO: 149 is an amino acid sequence of a fusion protein comprising CTLA-4-Fc-linker-BCMA-linker-BCMA-linker-BCMA;
SEQ ID NO: 150 is an amino acid sequence of a fusion protein comprising TNFR2-Fc-linker-BAFFR-linker-BCMA;
SEQ ID NO: 151 is an amino acid sequence of a fusion protein comprising Ox40R-Fc-linker-BCMA;
SEQ ID NO: 152 is an amino acid sequence of a fusion protein comprising IFNGR1-Fc-linker-BCMA;
SEQ ID NO: 153 is an amino acid sequence of a fusion protein comprising ICOSR-Fc-linker-BCMA-linker-BAFFR;
SEQ ID NO: 154 is an amino acid sequence of a fusion protein comprising IFNAR2 (high affinity)-linker-BAFFR;
SEQ ID NO: 155 is an amino acid sequence of a fusion protein comprising IL21R-Gamma Chain-Fc-linker-BAFFR;
SEQ ID NO: 156 is an amino acid sequence of a fusion protein comprising IL-17 RA-Fc/IgG1-BAFFR;
SEQ ID NO: 157 is an amino acid sequence of a fusion protein comprising IL-17 RA-Fc/IgG1-linker-BAFFR;
SEQ ID NO: 158 is an amino acid sequence of a fusion protein comprising IL-17 RA-Fc/IgG1-linker-BAFFR;
SEQ ID NO: 159 is an amino acid sequence of a fusion protein comprising IL-17 RA-Fc/IgG1-linker-BAFFR;
SEQ ID NO: 160 is an amino acid sequence of a fusion protein comprising IL-17 RA-Fc/IgG1-linker-BAFFR;
SEQ ID NO: 161 is an amino acid sequence of a fusion protein comprising IL-17 RA-Fc/IgG1-linker-BAFFR;
SEQ ID NO: 162 is an amino acid sequence of a fusion protein comprising IL-17 RA-Fc/IgG1-linker-BAFFR;
SEQ ID NO: 163 is an amino acid sequence of a fusion protein comprising IL-17 RA-Fc/IgG1-linker-BAFFR;
SEQ ID NO: 164 is an amino acid sequence of a fusion protein comprising IL-17 RA-Fc/IgG4-linker-BAFFR;
SEQ ID NO: 165 is an amino acid sequence of a fusion protein comprising BCMA-linker-BCMA-linker-BCMA-Fc/IgG4-linker-BAFFR-linker-BAFFR-linker-BAFFR;
SEQ ID NO: 166 is an amino acid sequence of a fusion protein comprising BCMA-linker-BCMA-Fc/IgG1-BAFFR-linker-BAFFR-linker-BAFFR;
SEQ ID NO: 167 is an amino acid sequence of a fusion protein comprising CD40-Fc-linker-BAFFR;
SEQ ID NO: 168 is an amino acid sequence of a fusion protein comprising CD40-Fc-linker-BAFFR-linker-BAFFR;
SEQ ID NO: 169 is an amino acid sequence of a fusion protein comprising CD40-Fc-linker-BAFFR-linker-BAFFR-linker-BAFFR;
SEQ ID NO: 170 is an amino acid sequence of a fusion protein comprising CD40-Fc-linker-BAFFR-BCMA;
SEQ ID NO: 171 is an amino acid sequence of a fusion protein comprising CD40-Fc-linker-BCMA;
SEQ ID NO: 172 is an amino acid sequence of a fusion protein comprising CD40-Fc-linker-BCMA-linker-BCMA;
SEQ ID NO: 173 is an amino acid sequence of a fusion protein comprising CD40-Fc-linker-BCMA-linker-BCMA-linker-BCMA;
SEQ ID NO: 174 is an amino acid sequence of a fusion protein comprising CD40-Fc-linker-BAFFR-linker-BCMA;
SEQ ID NO: 175 is an amino acid sequence of a fusion protein comprising CD40-Fc-linker-BCMA;
SEQ ID NO: 176 is an amino acid sequence of a fusion protein comprising CD40-Fc-linker-BCMA-linker-BCMA;
SEQ ID NO: 177 is an amino acid sequence of a fusion protein comprising CD40-Fc-linker-BCMA-linker-BCMA-linker-BCMA;
SEQ ID NO: 178 is an amino acid sequence of a fusion protein comprising IL21R-linker-Common Gamma Chain-Fc-linker-BAFFR;
SEQ ID NO: 179 is an amino acid sequence of a fusion protein comprising IL23R-linker-IL12R Beta-1-Fc-BAFFR-linker-BAFFR;
SEQ ID NO: 180 is an amino acid sequence of a fusion protein comprising Extracellular Domains Of Soluble Pd-1 Receptor-FcIgG4-Linker-A1fibercept;
SEQ ID NO: 181 is an amino acid sequence of a fusion protein comprising an Extracellular Domain Of BDCA2-FcIgG4-linker-Recombinant BAFFR; and
SEQ ID NO: 182 is an amino acid sequence of a fusion protein comprising an extracellular domain of BDCA2-FCIGG4-linker-Recombinant CD40.

### Antibody-Soluble Receptor Fusion Proteins (Including Extracellular Domains)

SEQ ID NOS: 183-184 are amino acid sequences of a fusion protein comprising SECUKINUMAB (COSENTYX)-linker-BAFFR-linker-BAFFR;
SEQ ID NOS: 185-186 are amino acid sequences of a fusion protein comprising SECUKINUMAB (COSENTYX)-linker-BCMA-linker-BCMA-linker-BCMA;
SEQ ID NOS: 187-188 are amino acid sequences of a fusion protein comprising ADALIMUMAB/HUMIRA-linker-TACI-linker-BAFFR;
SEQ ID NOS: 189-190 are amino acid sequences of a fusion protein comprising PEMOBROLIZUMAB-linker-Soluble Recombinant VEGFR;
SEQ ID NOS: 191-194 are amino acid sequences of a fusion protein comprising MOSUNETUZUMAB (Bispecific CD20/CD3 Antibody)-linker-BAFFR-linker-BAFFR;
SEQ ID NOS: 195-196 are amino acid sequences of a fusion protein comprising ANTI-IFN-TYPE 1 Receptor Antibody-linker-BAFFR-linker-BAFFR-linker-BAFFR;
SEQ ID NOS: 197-198 are amino acid sequences of a fusion protein comprising RITUXIMAB (ANTI CD20)-linker-BAFFR-linker-BAFFR;
SEQ ID NOS: 199-200 are amino acid sequences of a fusion protein comprising SARILUMAZB (ANTI IL-6R ALPHA)-linker-BAFFR-linker-BAFFR;
SEQ ID NOS: 201-202 are amino acid sequences of a fusion protein comprising TAFASITAMAB (ANTI-CD19)-linker-BAFFR-linker-BAFFR;
SEQ ID NOS: 203-204 amino acid sequences of a fusion protein comprising BELIMUMAB-linker-BAFFR-linker-BAFFR;
SEQ ID NOS: 205-206 are amino acid sequences of a fusion protein comprising RITUXIMAB (ANTI CD20)-linker-BAFFR;
SEQ ID NOS: 207-208 are amino acid sequences of a fusion protein comprising RITUXIMAB (ANTI CD20)-linker-BAFFR-linker-BAFFR-linker-BAFFR;
SEQ ID NOS: 209-210 are amino acid sequences of a fusion protein comprising RITUXIMAB (ANTI CD20)-linker-TACI;
SEQ ID NOS: 211-212 are amino acid sequences of a fusion protein comprising RITUXIMAB (ANTI CD20)-linker-TACI-linker-TACI;
SEQ ID NO: 213-214 are amino acid sequences of a fusion protein comprising RITUXIMAB (ANTI CD20)-linker-TACI-linker-TACI-linker-TACI;
SEQ ID NO: 215-216 are amino acid sequences of a fusion protein comprising RITUXIMAB (ANTI CD20)-linker-BCMA;
SEQ ID NO: 217-218 are amino acid sequences of a fusion protein comprising RITUXIMAB (ANTI CD20)-linker-BCMA-linker-BCMA;
SEQ ID NO: 219-220 are amino acid sequences of a fusion protein comprising RITUXIMAB (ANTI CD20)-linker-BCMA-linker-BCMA-linker-BCMA;
SEQ ID NO: 221-222 are amino acid sequences of a fusion protein comprising RITUXIMAB (ANTI CD20)-linker-BAFFR-linker-TACI;
SEQ ID NOS: 223-224 are amino acid sequences of a fusion protein comprising Belimumab-BAFFR;
SEQ ID NOS: 225-226 are amino acid sequences of a fusion protein comprising Belimumab-TACI;
SEQ ID NOS: 227-228 are amino acid sequences of a fusion protein comprising Belimumab-BCMA;
SEQ ID NOS: 229-230 are amino acid sequences of a fusion protein comprising Belimumab-BAFFR-TACI;
SEQ ID NOS: 231-232 are amino acid sequences of a fusion protein comprising Secukinumab-soluble receptor IL-17RC
SEQ ID NOS: 233-234 are amino acid sequences of a fusion protein comprising TEPLIZUMAB-LINKER-TACI; and
SEQ ID NOS: 235-236 are amino acid sequences of a fusion protein comprising LECANEMAB-FC-IL-10.

### Additional Fusion Protein Sequences

SEQ ID NO: 237 is an amino acid sequence of a fusion protein comprising IL-2-Fc-linker-BAFFR;
SEQ ID NO: 238 is an amino acid sequence of a fusion protein comprising IL2-Fc-linker-BAFFR-linker BAFFR;
SEQ ID NO: 239 is an amino acid sequence of a fusion protein comprising IL10-Fc-linker-TNFR2;
SEQ ID NO: 240 is an amino acid sequence of a fusion protein comprising IL10-Fc-LINKER-IL17RA;
SEQ ID NO: 241 is an amino acid sequence of a fusion protein comprising IL10-Fc-linker-BAFFR-LINKER-BAFFR;
SEQ ID NO: 242 is an amino acid sequence of a fusion protein comprising IL10-Fc-LINKER-IL-2;
SEQ ID NO: 243 is an amino acid sequence of a fusion protein comprising IL17RA-LINKER-AN ALBUMIN BINDING SITE-LINKER-BAFFR; and
SEQ ID NO: 244 is an amino acid sequence of a fusion protein comprising CD40-AN ALBUMIN BINDING SITE-LINKER BAFFR-LINKER-BAFFR.
   Bispecific Antibody Fusion Proteins with Cytokine Ligand Binding Domain
SEQ ID NOS: 245-248 are amino acid sequences of a fusion protein comprising Teclistamab with human IL-2; and
SEQ ID NOS: 249-252 are amino acid sequences of a fusion protein comprising Teclistamab with human Interferon Gamma.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Sequences described herein are described with reference to GENBANK® accession numbers and UNIPROTKB and/or SWISSPROT identification numbers. The sequences cross-referenced in the GENBANK® and UNIPROTKB/SWISSPROT databases are expressly incorporated by reference as are equivalent and related sequences present in GENBANK®, UNIPROTKB/SWISSPROT, or other public databases. Also expressly incorporated herein by reference are all annotations present in the GENBANK® and UNIPROTKB/SWISSPROT databases associated with the sequences disclosed herein. Unless otherwise indicated or apparent the references to the GENBANK® database and the UNIPROTKB/SWISSPROT database are references to the most recent version of the database as of the filing date of this Application.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The term “isolated”, when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high performance liquid chromatography. A polypeptide that is the predominant species present in a preparation is substantially isolated. The term “isolated” further denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment,” when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 20, 30, 40 or 50 amino acids long, at least 75 amino acids long, or at least 100, 150, 200, 300, 500 or more amino acids long.

The terms “N-terminus” or “amino-terminus” and “C-terminus” or “carboxyl-terminus” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide. Where amino-terminus or carboxyl-terminus refer to an entire polypeptide or polypeptide fragment, the terms refer to one or more amino acids at amino or carboxyl ends, respectively, of the polypeptide or the polypeptide fragment.

Polypeptide fragments can also be inclusive of “functional fragments,” in which case the fragment retains some or all of the activity of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

The term “variant,” as used herein, refers to an amino acid sequence that is different from the reference polypeptide by one or more amino acids. In some embodiments, a variant polypeptide may differ from a reference polypeptide by one or more amino acid substitutions. For example, a polypeptide variant can differ from the reference polypeptide by one or more amino acid substitutions, i.e., mutations. In this regard, polypeptide variants comprising combinations of two or more mutations can respectively be referred to as double mutants, triple mutants, and so forth. It will be recognized that certain mutations can result in a notable change in function of a polypeptide, while other mutations will result in little to no notable change in function of the polypeptide. In this regards, in some embodiments, such polypeptide variants can thus also be inclusive of “functional variants,” in which case the variant retains some or all of the activity of the reference polypeptide.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified variants thereof (e.g., degenerate variants, including degenerate codon substitutions) and complementary sequences and as well as the sequence explicitly indicated.

The term “degenerate variant” refers to a nucleic acid having a residue sequence that differs from a reference nucleic acid by one or more degenerate codon substitutions. Degenerate codon substitutions can be achieved by generating sequences in which the third position of one or more selected (or all) codons is substituted with mixed base and/or deoxyinosine residues (Batzer, et al. (1991) Nucleic Acid Res 19:5081; Ohtsuka et al. (1985) J Biol Chem 260:2605 2608; Rossolini et al. (1994) Mol Cell Probes 8:91 98).

The presently-disclosed subject matter includes compositions and methods for treatment of Sjögren's Syndrome and/or Systemic Lupus Erythematosus. In particular, certain embodiments of the presently-disclosed subject matter relate to fusion proteins including multiple ligand binding domains for targeting and/or binding one or more cytokines, growth factors, hormones, enzymes, cell surface receptors, or other proteins associated with Sjögren's Syndrome and/or Systemic Lupus Erythematosus pathology.

One aspect of the presently-disclosed subject matter thus pertains to fusion proteins and nucleic acids (e.g., DNA) encoding the fusion proteins. The term “fusion protein” is intended to describe at least two polypeptides, typically from different sources, which are operatively linked. With regard to the polypeptides, the term “operatively linked” is intended to mean that the two polypeptides are connected in a manner such that each polypeptide can serve its intended function. Typically, the two polypeptides are covalently attached through peptide bonds and can be produced by standard recombinant or chemical synthesis techniques. For example, as described in further detail below and by using recombinant techniques, a DNA molecule encoding a first polypeptide can be ligated to another DNA molecule encoding the second polypeptide, either directly or through the use of a further nucleic acid encoding an additional peptide sequence (e.g., a linker peptide), and the resultant hybrid DNA molecule can be expressed in a host cell to produce the fusion protein. The DNA molecules are generally ligated to each other in a 5′ to 3′ orientation such that, after ligation, the translational frame of the encoded polypeptides is not altered (i.e., the DNA molecules are ligated to each other in-frame). In certain embodiments, the nucleic acid sequences encoding the two or more peptides can be chemically-synthesized together or connected using polymerase chain reaction (PCR).

In some embodiments, the fusion polypeptides of the presently-disclosed subject matter are comprised, at least in part, of a fragment crystallizable (Fe) region of immunoglobulin G (IgG) and at least two ligand binding domains that are each individually capable of binding one or more cytokines, growth factors, hormones, enzymes, cell surface receptors, or other proteins associated with Sjögren's Syndrome and/or Systemic Lupus Erythematosus pathology. In this regard, the term “ligand binding domain” is used herein to refer to a domain or portion of a protein which binds to a cytokine, growth factor, hormone, enzyme, cell surface receptor, or other proteins or other protein ligand of interest. For example, in certain embodiments, the term “ligand binding domain” is used herein to refer to the portion of a cytokine or other protein receptor through which the binding of a cytokine or other protein typically occurs. In this regard, in some embodiments, reference to a “ligand binding domain” can refer to only that portion of the full-length receptor protein capable of binding to a particular cytokine or can refer to the full-length extracellular receptor protein.

For example, in some embodiments of the presently-disclosed subject matter, the ligand binding domain included in an exemplary fusion protein is an IL-17A receptor (CD217; UniProtKB No. Q96f46), or a fragment or variant thereof. In some embodiments, the ligand binding domain is comprised of the extracellular domain of the full-length IL-17A receptor (SEQ ID NO: 1) to thereby produce a fusion protein including a more soluble ligand binding domain and which includes a number of potential glycosylation sites. In some embodiments and without wishing to be bound by any particular theory or mechanism, it is contemplated that the signal peptide of the IL-17A receptor can further be replaced with a signal from another efficiently secreted protein to thereby promote the secretion of an exemplary fusion protein of the presently-disclosed subject matter.

As another example of a ligand binding domain capable of use in an exemplary fusion protein, in some embodiments, the ligand binding domain comprises a BAFF receptor (gene: TNFRSF13C; UniProtKB No. Q5H8V1), or a fragment or variant thereof, and which is also referred to as a Tall-1 or BlyS receptor. In some embodiments, the BAFF receptor ligand binding domain comprises the binding domain of the BAFF receptor (SEQ ID NO: 2). In some embodiments, the BAFF receptor ligand binding domain comprises a fragment of the full-length BAFF receptor and can be characterized as a soluble BAFF receptor ligand binding domain, such as the soluble BAFF receptor ligand binding domain included in SEQ ID NO: 3, SEQ ID NO: 4, SEQ ID NO: 5, SEQ ID NO: 6, or SEQ ID NO: 7. In further embodiments, two BAFF receptor ligand binding domains having the amino acid of SEQ ID NO: 8 are used and which includes two BAFF receptor ligand binding domains connected to one another with a repeating flexible linker peptide (e.g., GGGGS (SEQ ID NO: 43)). In some embodiments, the repeating flexible linker peptide can be provided in a number of repeats (e.g., (GGGGS)n (SEQ ID NO: 43) where n=1 to 20 or (GGGS)n (SEQ ID NO: 70) where n=1 to 20). Of course, in some embodiments and as described in further detail below, other linker peptides may also be used (e.g., non-immunogenic and/or charged) in order to improve the solubility or allow the proper folding of the domains of a fusion protein of the presently-disclosed subject matter.

As yet another example of a ligand binding domain capable of use in an exemplary fusion protein, in some embodiments, the ligand binding domain comprises an IFN-γ receptor, which includes the IFN-γ receptor 1 (IFNGR1) and IFN-γ receptor 2 (IFNGR2). In some embodiments, the IFN-γ receptor ligand binding domain is comprised IFNGR1 (UniProtKB No. P15260), or a fragment or a variant thereof. In some embodiments, the IFN-γ receptor ligand binding domain is comprised of the extracellular binding domain of the IFGR1 (SEQ ID NO: 9) or a fragment or variant thereof.

As a further example of a ligand binding domain capable of use in an exemplary fusion protein, in some embodiments, the ligand binding domain comprises a Tumor Necrosis Factor Receptor or a fragment or variant thereof. In some embodiments, the ligand binding domain comprises a Tumor Necrosis Factor Receptor I (TNFR-I; UniProtKB NO. P19438), or a fragment or variant thereof. In some embodiments, the ligand binding domain comprises an extracellular domain of the TNFR-I having the sequence of SEQ ID NO: 10).

In some embodiments, the ligand binding domain comprises a Tumor Necrosis Factor Receptor-2 (UniProtKB Nos.: P20333, BIAJZ3, Q16042, Q6YI29, Q9UIH; also referred to as TNFR-2, TNFR-II, TNFR P75, TNFR1B;), or a fragment or variant thereof. In some embodiments, the ligand binding domain comprises an extracellular domain of the TNFR-2 having the sequence of SEQ ID NO: 11.

In some embodiments of the fusion proteins described herein that make use of a Tumor Necrosis Factor Receptor as a ligand binding domain, the ligand binding domain comprises a Tumor Necrosis Factor Receptor Superfamily Member 5 (UniProtKB Nos: P25942; also referred to as CD40 or TNFR5) or a fragment or variant thereof. In some embodiments, the ligand binding domain comprises an extracellular domain of the TNFR5 having the sequence of SEQ ID NO: 12.

In some embodiments, the ligand binding domain comprises a Tumor Necrosis Factor Receptor Superfamily Member 13B or TACI (Transmembrane activator and calcium-modulator and cyclophilin ligand (CAML) interactor) receptor (UniProtKB No.: 014836; also referred to as CD267) or a fragment or variant thereof. In some embodiments, the ligand binding domain comprises an extracellular domain of the TACI receptor having the sequence of SEQ ID NO: 13.

As a further example of a ligand binding domain capable of use in an exemplary fusion protein, in some embodiments the ligand binding domain comprises an Interferon Alpha/Beta Receptor 1 (IFNAR1) or a fragment or variant thereof. In some embodiments, the ligand binding domain comprises IFNAR1 (UniProtKB No. P17181), or a fragment or variant thereof, and which functions in general as a heterodimer with Interferon Alpha/Beta Receptor 2 (IFNAR2). In some embodiments, the ligand binding domain comprises an extracellular domain of the IFNAR1 having the sequence of SEQ ID NO: 14).

As another example of a ligand binding domain capable of use in an exemplary fusion protein, in some embodiments, the ligand binding domain comprises an Interferon Alpha/Beta Receptor 2 (IFNAR2) (UniProtKB No. P48551), which associates with IFNAR1 to form the plasma membrane receptor in the type I interferon signaling pathway. In some embodiments, the ligand binding domain comprises an extracellular domain of the IFNAR2 having the sequence of SEQ ID NO: 15).

As another example of a ligand binding domain capable of use in an exemplary fusion protein, in some embodiments, the ligand binding domain comprises an interleukin-21 (IL-21) receptor or a fragment or variant thereof. The interleukin-21 receptor forms a heterodimeric receptor complex with the common gamma-chain, a receptor subunit also shared by the receptors for interleukin 2 (IL-2), interleukin 7 (IL-7) and interleukin 15 (IL-15). This receptor transduces the growth promoting signal of IL-21, and is important for the proliferation and differentiation of T cells, B cells, and natural killer (NK) cells. In some embodiments, the ligand binding domain comprises an extracellular domain of IL-21 having the sequence of SEQ ID NO: 16).

As another example of a ligand binding domain capable of use in an exemplary fusion protein, in some embodiments, the ligand binding domain comprises the cytokine receptor common subunit gamma, which is a common subunit for the receptors for a variety of interleukins. In some embodiments, the ligand binding domain comprises the extracellular domain of cytokine receptor common subunit gamma having the sequence of SEQ ID NO: 17).

As another example of a ligand binding domain capable of use in an exemplary fusion protein, in some embodiments, the ligand binding domain comprises the cytotoxic T-lymphocyte protein 4 (SEQ ID NO: 18), which is protein receptor that often functions as an immune checkpoint and downregulates immune responses. In some embodiments, the ligand binding domain comprises tumor necrosis factor superfamily member 4 (TNFR4; SEQ ID NO: 19, which is also referred to as CD134 or the OX40L Receptor). In some embodiments, the ligand binding domain comprises the Inducible T-Cell Costimulatory Receptor (ICOS-R; SEQ ID NO: 20, which is also referred to as CD278). In some embodiments, the ligand binding domain comprises the CD27 Antigen Receptor (SEQ ID NO: 21).

In some embodiments, the ligand binding domain comprises a human IL-23R (IL-23R; UniProtKB: Q5VWK5; SEQ ID NO: 22) as it is appreciated that interleukin-23 is an important cytokine for autoimmune inflammation, and as it is further appreciated that the functional receptor for IL-23 (the IL-23 receptor) consists of a heterodimer. The heterodimeric cytokine IL-23 is formed by IL-12p40 and IL-23p19, and binds the IL-23 receptor complex comprised of IL-12RB1 (SEQ ID NO: 23) and IL-23R.

In some embodiments, the ligand binding domain comprises a human IL-21 receptor binding domain 1 (IL21R; UniProtKB: Q9HBE5; UniProtKB: P31785; SEQ ID NO: 24), which belongs to the type I cytokine receptors, and has been shown to form a heterodimeric receptor complex with the common gamma-chain that is shared by the receptors for interleukin-2, -4, -7, -9, -15, and IL-21. In some embodiments, the ligand binding domain comprises a Tumor Necrosis Factor Superfamily Member 9 (TNFR9; SEQ ID NO: 25).

Regardless of the particular ligand binding domain used in accordance with the presently-disclosed subject matter, as indicated above and in addition to including at least two ligand binding domains in an exemplary fusion polypeptide, the fusion polypeptides additionally include and generally make use of a fragment crystallizable or Fc region of immunoglobulin G (IgG) or a fragment or variant thereof. In some embodiments, fusion polypeptides of the presently-disclosed subject matter comprise two ligand binding domains and an Fc region such that the fusion polypeptides typically dimerize during cellular expression.

In some embodiments, the inclusion of such Fc regions not only improves the pharmacokinetic properties of the fusion proteins while increasing serum half-life, but further improves or maintains the efficacy of the ligand binding domains included in an exemplary fusion protein. Fc regions of IgG capable of use in accordance with the presently-disclosed subject matter can be derived from several IgG classes or carry mutations to obtain desired functions, and can be matched with a particular ligand binding domain as may be desired for a particular application. In some embodiments, IgG1, IgG2, and IgG4 are often preferred to IgG3 due to their longer half-lives of approximately three weeks, but it is of course recognized that IgG subtypes differ on their ability to exert effector functions depending on the binding affinity of IgG to FcγRs. For instance, while IgG1 and IgG3 have the highest binding affinity and, therefore, are more cytotoxic, the binding affinity of IgG4 is approximately 10-fold less than the affinity of IgG1 and IgG3, and the binding affinity of IgG2 is undetectable.

In some embodiments of the presently-disclosed subject matter and with further regard to the Fc regions included in an exemplary fusion proteins of the presently-disclosed subject matter, the Fc region is selected from the Fc region of wild-type IgG1, IgG2, IgG3, IgG4 and can include the CH2 and CH3 regions, the CH2 region only, or the CH3 region only of those immunoglobulins to thereby modulate the half-life of the fusion proteins. In some embodiments, and as indicated above, the Fc regions included in an exemplary fusion protein are capable of forming heterodimers, where two different Fc regions are each linked to one or more ligand binding domains, but form a heterodimer molecule through the formation of disulfide bridges and thereby form a single molecule including two different heterodimer regions and multiple (e.g., 4 or more) ligand binding domains, as described in further detail below.

In some embodiments, one or more mutations can be further introduced into the Fc region, such as mutations that result in stronger binding to the neonatal Fc receptor (FcRn) and longer half-life (e.g., for IgG1) or mutations that decrease or increase effector functions such as complement functions and ADCC (antibody-dependent cell-mediated cytotoxicity; i.e., a cytotoxic reaction in which FcR-bearing killer cells recognize target cells via specific antibodies). Moreover, in some embodiments, the Fc portion of the fusion polypeptides of the presently-disclosed subject matter can thus be regarded as a variable part of the constructs that can be derived from several IgG classes or that can carry mutations so as to obtain desired functions. For instance, in some embodiments, Fc portions derived from IgG1, IgG2 and IgG4 can be preferred over IgG3 due to their longer half-lives of approximately three weeks. In other embodiments, however, an Fc portion may be chosen based on a desired binding affinity as it is appreciated that IgG subtypes differ on their ability to exert effector functions depending on the binding affinity of IgG to FcγR, with IgG1 and IgG3 having the highest binding affinity and consequently being more cytotoxic, and with the binding affinity of IgG4 being approximately 10-fold less than the affinity of IgG1 and IgG3, while binding affinity of IgG2 is close to undetectable.

In some embodiments, the Fc region included in an exemplary fusion protein is an Fc IgG1 heavy chain constant region variant having the sequence of SEQ ID NO: 26 or SEQ ID NO: 27. In other embodiments, the Fc region included in an exemplary fusion protein is a variant of an Fc IgG1 Heavy Chain Constant Region having the sequence of SEQ ID NO: 28, which includes the mutations M428L and N434S, such that the variant Fc region exhibits stronger binding to the neonatal FC Receptor (FCRN) and has a longer half-life.

In some embodiments, the Fc region is a variant Fc IgG1 Heavy Chain Constant Region with mutations that reduce effector functions. In some embodiments, such a variant Fe region having mutations that reduce effector function has the sequence of SEQ ID NO: 29. In other embodiments, such a variant Fc region having mutations that reduce effector function has the sequence of SEQ ID NO: 30, which includes the mutations N279A, N297Q, and N297G to thereby reduce effector functions. In other embodiments, it is alternatively contemplated that mutations may also be introduced in Fc regions to increase effector function, such as the variant Fc IgG1 Heavy Chain Constant Region provided by SEQ ID NO: 31.

With further regard to the Fc regions included in an exemplary fusion polypeptide, the presently-disclosed subject is further inclusive of variant Fc IgG1 Heavy Chain Constant Regions having variable effector properties and/or other functional characteristics. In some embodiments, such additional Fc variants have an amino acid sequence selected from the group consisting of SEQ ID NOS: 32-42. Of course, it is also contemplated that, in some embodiments, other transport or carrier sequences may further be included in an exemplary fusion polypeptide, such as albumin-based peptides, without departing from the spirit and scope of the subject matter described herein in order to modulate the properties of the fusion proteins

Regardless of the particular ligand binding domains and Fc or other regions included in an exemplary fusion proteins, and as noted above and described in further detail below, to operatively link the ligand binding domains and Fc or other regions to one another, nucleotide sequences encoding the ligand binding domains and Fc or other regions are generally ligated to each other in-frame to create a chimeric gene encoding an exemplary fusion polypeptide. In some embodiments, however, a further nucleic acid sequence encoding an additional polypeptide sequence can be incorporated between the nucleotide sequences encoding the ligand binding domains and Fe or other regions. For example, in some embodiments, a fusion polypeptide can be provided that contains an operatively-linked polypeptide in the form of a linker peptide to connect the ligand binding domains to each other and/or to the Fc or other region of the fusion polypeptide.

In some embodiments, such linker peptides can include amino acid sequences of various lengths, such as, for example, 4-6 amino acids, 7-12, 13-25, 26-50, or even more amino acid residues. In some embodiments, the linker peptides are about 1 to about 30 amino acids in length. These added linker peptide sequences can be flexible or rigid (e.g., through the use linker peptides having an alpha helix confirmation), allowing portions of the fusion protein to interact with one another or to only be connected via the linker peptide, or to allow the portions of the fusion proteins to fold properly/individually without interference. In some embodiments, the linker peptides can also increase the functionality, or pharmacological properties, production, or purification yield. Further, in some embodiments, the linker peptides include disulfides, protease cleavage sites, or glycosylation sites (NXS or NXT), which can enhance the solubility of an exemplary fusion protein, reducing aggregation, or reduce antigenicity. In some embodiments, the linker peptide included between ligand binding domains of the presently-disclosed subject matter are or are derived from Fe sequences (e.g., hinge-CH2-CH3) from IgG1, IgG2, IgG3, and IgG4. In other embodiments, other proteins such as albumin and transferrin can also be used to produce an exemplary linker sequence capable of use herein. In some embodiments of the presently-disclosed subject matter, the linker peptides have a sequence selected from: SEQ ID NOS: 43-71.

With further regard to the exemplary fusion polypeptides described herein, in some embodiments, the exemplary fusion polypeptides further include a secreted protein hydrophobic leader sequence to encourage secretion of the fusion protein upon production.

By making use of the above-described ligand binding domains connected to an Fc region with or without the use of a linker peptide, in some embodiments of the presently-disclosed subject matter, fusion proteins can thus be provided having multiple different or alternative configurations of two or more ligand binding domains connected to an Fc region. In some embodiments, the fusion proteins produced in accordance with the presently-disclosed subject matter have the general formulas shown below, where the ligand binding domains are indicated as “LBD”, the Fc regions are indicated as “FC”, where the optional linker peptides are indicates as “-”, where antibody heavy chains are indicated as “HC”, where antibody light chains are indicated as “LC”, and where monoclonal antibody binding domains are indicated as “mAb”.

For example, in some embodiments, Fc fusion polypeptides are provided having the general formulas (I)-(VIII) as shown below where the fusion polypeptides can be described as dimeric and where the positions of the ligand binding domains can be switched from in front and behind the Fc portion of the molecule. In some embodiments, such constructs are produced from identical DNA constructs as described below, with the Fc sequences being engineered such that the two constructs/peptides form homodimers upon production

LBD1-Fc-LBD2 (I)

LBD1-Fc-LBD2-LBD2 (II)

LBD1-Fc-LBD2-LBD2-LBD2 (III)

LBD1-Fc-LBD2-LBD3 (IV)

LBD1A-LBD1B-Fc-LBD2 (V)

LBD1A-LBD1B-Fc-LBD2-LBD2 (VI)

LBD1A-LBD1B-Fc-LBD2-LBD2-LBD2 (VII)

LBD1A-LBD1B-Fc-LBD2-LBD3-LBD4 (VIII)

As another example, in some embodiments, fusion polypeptides are provided having the general formulas (IX)-(XVIII)) as shown below where the fusion polypeptides paired below (i.e., fusion polypeptides IX and X, XI and XII, XIII and XIV, XV and XVI, and XVII and XVIII) are produced from two DNA constructs as described below, but where the Fc sequences are engineered such that the two constructs/peptides form heterodimers upon production.

LBD1A-Fc1-LBD2 (IX)

LBD1B-Fc2-LBD2 (X)

LBD1A-Fc1-LBD2-LBD3 (XI)

LBD1B-Fc2-LBD2-LBD3 (XII)

LBD1A-Fel-LBD2A (XIII)

LBD1B-Fc2-LBD2B (XIV)

LBD1A-Fel-LBD2-LBD2 (XV)

LBD1B-Fc2-LBD2-LBD2 (XVI)

LBD1A-Fc1-LBD2-LBD2-LBD2 (XVII)

LBD1B-Fc2-LBD2-LBD2-LBD2 (XVIII)

As another example, in some embodiments, fusion polypeptides are provided having the general formulas (IXX)-(XXIII) as shown below, where the fusion polypeptides are comprised of antibodies and ligand binding domains, where the antibodies include the Fc region and where antibodies are produced from two nucleic acid sequences with one sequence encoding the heavy chain with the receptors and with the other sequence encoding the light chain.

mAb-LBD1 (IXX)

mAb-LBD1-LBD1 (XX)

mAb-LBD1-LBD1-LBD1 (XXI)

mAb-LBD1-LBD2 (XXII)

mAb-LBD1-LBD2-LBD3 (XXIII)

As another example, in some embodiments, fusion polypeptides are provided having the general formulas (XXIV)-(XLIII) as shown below where the polypeptides paired below (i.e., polypeptides (XXIV)-(XXVII), (XXVIII)-(XXXI), (XXXII)-(XXXV), (XXXVI)-(XXXIX), and (XL)-(XLIII)) are comprised of bi-specific monoclonal antibodies having two different binding domains in the heavy chains, where the Fc region is engineered to form heterodimers, where there are 2 light chains, and where there can be 4 discreet genes included in a plasmid that, for example, result in 3 binding sites. In polypeptides (XXXVI)-(XXXIX), 4 binding sites are included. In polypeptides (XL)-(XLIII), 3 binding sites are included and one polypeptide is a soluble heterodimeric receptor where the Fe regions differ and include HC1 and HC2.

HC1-LBD1 (XXIV)

HC2-LBD1 (XXV)

LC1 (XXVI)

LC2 (XXVII)

HC1-LBD1-LBD1 (XXVIII)

HC2-LBD1-LBD1 (XXIX)

LC1 (XXX)

LC2 (XXXI)

HC1-LBD1-LBD1-LBD1 (XXXII)

HC2-LBD1-LBD1-LBD1 (XXXIII)

LC1 (XXXIV)

LC2 (XXXV)

HC1-LBD1-LBD2 (XXXVI)

HC2-LBD1-LBD2 (XXXVII)

LC1 (XXXVIII)

LC2 (XXXIX)

HC1-LBD1A (XL)

HC2-LBD1B (XLI)

LC1 (XLII)

LC2 (XLIII)

In some embodiments, an exemplary fusion protein comprises a sequence selected from the group consisting of SEQ ID NOS: 72-182, which are discussed in further detail below and where it is understood, for example, that reference to a particular receptor is a reference to a peptide comprising a ligand binding domain from that receptor.

In some embodiments, an exemplary fusion protein is provided in which the fusion protein comprises the sequence of SEQ ID NO: 72, wherein the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 73, where the fusion protein is comprised of IL-17 RA operatively linked to a Fc/IgG1 region operatively linked to BAFFR operatively linked to a further BAFFR.

As another example, in some embodiments, an exemplary fusion protein is provided in which the fusion protein comprises the sequence of SEQ ID NO: 74, where the fusion protein is comprised of IL-17 RA operatively linked to a Fc/IgG1 region operatively linked to BAFFR operatively linked to BAFFR operatively linked to BAFFR.

As another example, in some embodiments, an exemplary fusion protein is provided in which the fusion protein comprises the sequence of SEQ ID NO: 75, where the fusion protein is comprised of IL-17 RA operatively linked to a Fc region operatively linked to IFN gamma RI.

As another example, in some embodiments, an exemplary fusion protein is provided in which the fusion protein comprises the sequence of SEQ ID NO: 76, where the fusion protein is comprised of TNFR2 operatively linked to a Fc region operatively linked to BAFFR operatively linked to BAFFR.

As another example, in some embodiments, an exemplary fusion protein is provided in which the fusion protein comprises the sequence of SEQ ID NO: 77, where the fusion protein is comprised of TNFR5 operatively linked to a Fc region operatively linked to BAFFR operatively linked to BAFFR.

As another example, in some embodiments, an exemplary fusion protein is provided in which the fusion protein comprises the sequence of SEQ ID NO: 78, where the fusion protein is comprised of IFN gammaR operatively linked to a Fc region operatively linked to BCMA.

As another example, in some embodiments, an exemplary fusion protein is provided in which the fusion protein comprises the sequence of SEQ ID NO: 79, where the fusion protein is comprised of IL-17 RA operatively linked to a Fc/IgG1 region operatively linked to TACI.

As another example, in some embodiments, an exemplary fusion protein is provided in which the fusion protein comprises the sequence of SEQ ID NO: 80 where the fusion protein is comprised of IL-17 RA operatively linked to a Fc/IgG1 region operatively linked to BCMA.

As another example, in some embodiments, an exemplary fusion protein is provided in which the fusion protein comprises the sequence of SEQ ID NO: 81 where the fusion protein is comprised of CTLA-4 operatively linked to a Fc region operatively linked to BAFFR.

As another example, in some embodiments, an exemplary fusion protein is provided in which the fusion protein comprises the sequence of SEQ ID NO: 82, where the fusion protein is comprised of CTLA-4 operatively linked to a Fc region operatively linked to BAFFR operatively linked to BAFFR.

As another example, in some embodiments, an exemplary fusion protein is provided in which the fusion protein comprises the sequence of SEQ ID NO: 83, where the fusion protein is comprised of CTLA-4 operatively linked to a Fc region operatively linked to BAFFR operatively linked to BAFFR operatively linked to BAFFR.

As another example, in some embodiments, an exemplary fusion protein is provided in which the fusion protein comprises the sequence of SEQ ID NO: 84, where the fusion protein is comprised of TNFR2 operatively linked to a Fc region operatively linked to BAFFR.

As another example, in some embodiments, an exemplary fusion protein is provided in which the fusion protein comprises the sequence of SEQ ID NO: 85, where the fusion protein is comprised of Ox40R operatively linked to a Fe region operatively linked to BAFFR.

As another example, in some embodiments, an exemplary fusion protein is provided in which the fusion protein comprises the sequence of SEQ ID NO: 86, where the fusion protein is comprised of IFNGR1 operatively linked to a Fc region operatively linked to BAFFR.

As another example, in some embodiments, an exemplary fusion protein is provided in which the fusion protein comprises the sequence of SEQ ID NO: 87, where the fusion protein is comprised of ICOSR operatively linked to a Fc region operatively linked to BAFFR.

As another example, in some embodiments, an exemplary fusion protein is provided in which the fusion protein comprises the sequence of SEQ ID NO: 88, where the fusion protein is comprised of IL21R operatively linked to common gamma chain operatively linked to a Fc region operatively linked to BAFFR.

As another example, in some embodiments, an exemplary fusion protein is provided in which the fusion protein comprises the sequence of SEQ ID NO: 89, where the fusion protein is comprised of IL21R-gamma chain operatively linked to a Fc region operatively linked to BAFFR.

As another example, in some embodiments, an exemplary fusion protein is provided in which the fusion protein comprises the sequence of SEQ ID NO: 90, where the fusion protein is comprised of IL-17 RA operatively linked to a Fc/IgG1 region operatively linked to BAFFR operatively linked to TACI.

As another example, in some embodiments, an exemplary fusion protein is provided in which the fusion protein comprises the sequence of SEQ ID NO: 91, where the fusion protein is comprised of CTLA-4 operatively linked to a Fc region operatively linked to BAFFR operatively linked to BCMA.

As another example, in some embodiments, an exemplary fusion protein is provided in which the fusion protein comprises the sequence of SEQ ID NO: 92, where the fusion protein is comprised of CTLA-4 operatively linked to a Fc region operatively linked to BCMA.

As another example, in some embodiments, an exemplary fusion protein is provided in which the fusion protein comprises the sequence of SEQ ID NO: 93, where the fusion protein is comprised of CTLA-4 operatively linked to a Fc region operatively linked to BCMA operatively linked to BCMA.

As another example, in some embodiments, an exemplary fusion protein is provided in which the fusion protein comprises the sequence of SEQ ID NO: 94, where the fusion protein is comprised of CTLA-4 operatively linked to a Fc region operatively linked to BCMA operatively linked to BCMA operatively linked to BCMA.

As another example, in some embodiments, an exemplary fusion protein is provided in which the fusion protein comprises the sequence of SEQ ID NO: 95, where the fusion protein is comprised of CTLA-4 operatively linked to a Fc region operatively linked to BAFFR operatively linked to BAFFR.

As another example, in some embodiments, an exemplary fusion protein is provided in which the fusion protein comprises the sequence of SEQ ID NO: 96, where the fusion protein is comprised of TNFR2 operatively linked to a Fc region operatively linked to BAFFR operatively linked to BCMA.

As another example, in some embodiments, an exemplary fusion protein is provided in which the fusion protein comprises the sequence of SEQ ID NO: 97, where the fusion protein is comprised of Ox40R operatively linked to a Fc region operatively linked to BCMA.

As another example, in some embodiments, an exemplary fusion protein is provided in which the fusion protein comprises the sequence of SEQ ID NO: 98, where the fusion protein is comprised of IFNGR1 operatively linked to a Fc region operatively linked to BCMA.

As another example, in some embodiments, an exemplary fusion protein is provided in which the fusion protein comprises the sequence of SEQ ID NO: 99, where the fusion protein is comprised of ICOSR operatively linked to a Fc region operatively linked to BCMA operatively linked to BAFFR.

As another example, in some embodiments, an exemplary fusion protein is provided in which the fusion protein comprises the sequence of SEQ ID NO: 100, where the fusion protein is comprised of IFNAR2 (high affinity) operatively linked to a Fc region operatively linked to BAFFR.

As another example, in some embodiments, an exemplary fusion protein is provided in which the fusion protein comprises the sequence of SEQ ID NO: 101, where the fusion protein is comprised of IL21R operatively linked to gamma chain operatively linked to a Fc region operatively linked to BAFFR operatively linked to BAFFR.

As another example, in some embodiments, an exemplary fusion protein is provided in which the fusion protein comprises the sequence of SEQ ID NO: 102, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 103, where the fusion protein is comprised of IL-17 RA operatively linked to a Fc region operatively linked to BAFFR, which is connected via a linker to a second BAFFR.

As another example, in some embodiments, an exemplary fusion protein is provided in which the fusion protein comprises the sequence of SEQ ID NO: 104, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 105, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 106, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 107, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 108, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 109, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 110, where the fusion protein is comprised of IL-17 RA operatively linked to a Fc/IgG4 region operatively linked to BAFFR.

As another example, in some embodiments, an exemplary fusion protein is provided in which the fusion protein comprises the sequence of SEQ ID NO: 111, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 112, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 113, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 114, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 115, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 116, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 117, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 118, where the fusion protein is comprised of IL-17 RA operatively linked to a Fe/IgG1 region operatively linked to BAFFR operatively linked to BAFFR.

As another example, in some embodiments, an exemplary fusion protein is provided in which the fusion protein comprises the sequence of SEQ ID NO: 119, where the fusion protein is comprised of IL-17 RA operatively linked to a Fc/IgG1 region operatively linked to BAFFR operatively linked to BAFFR operatively linked to BAFFR.

As another example, in some embodiments, an exemplary fusion protein is provided in which the fusion protein comprises the sequence of SEQ ID NO: 120, where the fusion protein is comprised of BAFFR operatively linked to a Fc region operatively linked to BAFFR operatively linked to TACI.

As another example, in some embodiments, an exemplary fusion protein is provided in which the fusion protein comprises the sequence of SEQ ID NO: 121, where the fusion protein is comprised of IL-21R operatively linked to a Fc(1) region operatively linked to BAFFR.

As another example, in some embodiments, an exemplary fusion protein is provided in which the fusion protein comprises the sequence of SEQ ID NO: 122, where the fusion protein is comprised of IL22R alpha operatively linked to IL10R beta operatively linked to a Fc region operatively linked to BAFFR operatively linked to BAFFR.

As another example, in some embodiments, an exemplary fusion protein is provided in which the fusion protein comprises the sequence of SEQ ID NO: 123, where the fusion protein is comprised of PD-1 operatively linked to a Fc region operatively linked to CTLA-4.

As another example, in some embodiments, an exemplary fusion protein is provided in which the fusion protein comprises the sequence of SEQ ID NO: 124, where the fusion protein is comprised of LAG3 operatively linked to a Fc region operatively linked to CTLA-4.

As another example, in some embodiments, an exemplary fusion protein is provided in which the fusion protein comprises the sequence of SEQ ID NO: 125, where the fusion protein is comprised of TIM-3 extracellular domain operatively linked to a Fc region operatively linked to PD1.

As another example, in some embodiments, an exemplary fusion protein is provided in which the fusion protein comprises the sequence of SEQ ID NO: 126, where the fusion protein is comprised of soluble LAG-3 operatively linked to a Fc/IgG1 region operatively linked to PD1.

As another example, in some embodiments, an exemplary fusion protein is provided in which the fusion protein comprises the sequence of SEQ ID NO: 127, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 128, where the fusion protein is comprised of IL-17 RA operatively linked to a Fc/IgG1 region operatively linked to BAFFR operatively linked to BAFFR.

As another example, in some embodiments, an exemplary fusion protein is provided in which the fusion protein comprises the sequence of SEQ ID NO: 129, where the fusion protein is comprised of IL-17 RA operatively linked to a Fc/IgG1 region operatively linked to BAFFR operatively linked to BAFFR operatively linked to BAFFR.

As another example, in some embodiments, an exemplary fusion protein is provided in which the fusion protein comprises the sequence of SEQ ID NO: 130, where the fusion protein is comprised of IL-17 AR operatively linked to a Fc region operatively linked to IFN gamma RI.

As another example, in some embodiments, an exemplary fusion protein is provided in which the fusion protein comprises the sequence of SEQ ID NO: 131, where the fusion protein is comprised of TNFR2 operatively linked to a Fc region operatively linked to BAFFR operatively linked to BAFFR.

As another example, in some embodiments, an exemplary fusion protein is provided in which the fusion protein comprises the sequence of SEQ ID NO: 132, where the fusion protein is comprised of TNFR5 operatively linked to a Fc region operatively linked to BAFFR operatively linked to BAFFR.

As another example, in some embodiments, an exemplary fusion protein is provided in which the fusion protein comprises the sequence of SEQ ID NO: 133, where the fusion protein is comprised of IFN AR2 operatively linked to a Fc region operatively linked to BCMA.

As another example, in some embodiments, an exemplary fusion protein is provided in which the fusion protein comprises the sequence of SEQ ID NO: 134, where the fusion protein is comprised of IL-17 RA operatively linked to a Fc/IgG1 region operatively linked to TACI.

As another example, in some embodiments, an exemplary fusion protein is provided in which the fusion protein comprises the sequence of SEQ ID NO: 135, where the fusion protein is comprised of IL-17 RA operatively linked to a Fc/IgG1 region operatively linked to BCMA.

As another example, in some embodiments, an exemplary fusion protein is provided in which the fusion protein comprises the sequence of SEQ ID NO: 136, where the fusion protein is comprised of CTLA-4 operatively linked to a Fc region operatively linked to a BAFFR.

As another example, in some embodiments, an exemplary fusion protein is provided in which the fusion protein comprises the sequence of SEQ ID NO: 137, where the fusion protein is comprised of CTLA-4 operatively linked to a Fc region operatively linked to BAFFR operatively linked to BAFFR.

As another example, in some embodiments, an exemplary fusion protein is provided in which the fusion protein comprises the sequence of SEQ ID NO: 138, where the fusion protein is comprised of CTLA-4 operatively linked to a Fc region operatively linked to BAFFR operatively linked to BAFFR operatively linked to BAFFR.

As another example, in some embodiments, an exemplary fusion protein is provided in which the fusion protein comprises the sequence of SEQ ID NO: 139, where the fusion protein is comprised of TNFR2 operatively linked to a Fc region operatively linked to BAFFR.

As another example, in some embodiments, an exemplary fusion protein is provided in which the fusion protein comprises the sequence of SEQ ID NO: 140, where the fusion protein is comprised of Ox40R operatively linked to a Fc region operatively linked to BAFFR.

As another example, in some embodiments, an exemplary fusion protein is provided in which the fusion protein comprises the sequence of SEQ ID NO: 141, where the fusion protein is comprised of IFNGR1 operatively linked to a Fc region operatively linked to BAFFR.

As another example, in some embodiments, an exemplary fusion protein is provided in which the fusion protein comprises the sequence of SEQ ID NO: 142, where the fusion protein is comprised of ICOSR operatively linked to a Fc region operatively linked to BAFFR.

As another example, in some embodiments, an exemplary fusion protein is provided in which the fusion protein comprises the sequence of SEQ ID NO: 143, where the fusion protein is comprised of IFNAR1 operatively linked to IFNAR2 operatively linked to a Fc region operatively linked to BAFFR.

As another example, in some embodiments, an exemplary fusion protein is provided in which the fusion protein comprises the sequence of SEQ ID NO: 144, where the fusion protein is comprised of IL21R operatively linked to gamma chain operatively linked to a Fc region operatively linked to BCMA.

As another example, in some embodiments, an exemplary fusion protein is provided in which the fusion protein comprises the sequence of SEQ ID NO: 145, where the fusion protein is comprised of IL-17 RA operatively linked to a Fc/IgG1 region operatively linked to BAFFR operatively linked to TACI.

As another example, in some embodiments, an exemplary fusion protein is provided in which the fusion protein comprises the sequence of SEQ ID NO: 146, where the fusion protein is comprised of CTLA-4 operatively linked to a Fc region operatively linked to BAFFR operatively linked to BCMA.

As another example, in some embodiments, an exemplary fusion protein is provided in which the fusion protein comprises the sequence of SEQ ID NO: 147, where the fusion protein is comprised of CTLA-4 operatively linked to a Fc region operatively linked to BCMA.

As another example, in some embodiments, an exemplary fusion protein is provided in which the fusion protein comprises the sequence of SEQ ID NO: 148, where the fusion protein is comprised of CTLA-4 operatively linked to a Fe region operatively linked to BCMA operatively linked to BCMA.

As another example, in some embodiments, an exemplary fusion protein is provided in which the fusion protein comprises the sequence of SEQ ID NO: 149, where the fusion protein is comprised of CTLA-4 operatively linked to a Fc region operatively linked to BCMA operatively linked to BCMA operatively linked to BCMA.

As another example, in some embodiments, an exemplary fusion protein is provided in which the fusion protein comprises the sequence of SEQ ID NO: 150, where the fusion protein is comprised of TNFR2 operatively linked to a Fc region operatively linked to BAFFR operatively linked to BCMA.

As another example, in some embodiments, an exemplary fusion protein is provided in which the fusion protein comprises the sequence of SEQ ID NO: 151, where the fusion protein is comprised of Ox40R operatively linked to a Fc region operatively linked to BCMA.

As another example, in some embodiments, an exemplary fusion protein is provided in which the fusion protein comprises the sequence of SEQ ID NO: 152, where the fusion protein is comprised of IFNGR1 operatively linked to a Fc region operatively linked to BCMA.

As another example, in some embodiments, an exemplary fusion protein is provided in which the fusion protein comprises the sequence of SEQ ID NO: 153, where the fusion protein is comprised of ICOSR operatively linked to a Fc region operatively linked to BCMA operatively linked to BAFFR.

As another example, in some embodiments, an exemplary fusion protein is provided in which the fusion protein comprises the sequence of SEQ ID NO: 154, where the fusion protein is comprised of IFNAR2 (high affinity) operatively linked to BAFFR.

As another example, in some embodiments, an exemplary fusion protein is provided in which the fusion protein comprises the sequence of SEQ ID NO: 155, where the fusion protein is comprised of IL21R operatively linked to gamma chain operatively linked to a Fc region operatively linked to BAFFR.

As another example, in some embodiments, an exemplary fusion protein is provided in which the fusion protein comprises the sequence of SEQ ID NO:156, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 157, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 158, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 159, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 160, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 161, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 162, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 163, where the fusion protein is comprised of IL-17 RA operatively linked to a Fc/IgG1 region operatively linked to BAFFR.

As another example, in some embodiments, an exemplary fusion protein is provided in which the fusion protein comprises the sequence of SEQ ID NO: 164, where the fusion protein is comprised of IL-17 RA operatively linked to a Fc/IgG4 region operatively linked to BAFFR.

As another example, in some embodiments, an exemplary fusion protein is provided in which the fusion protein comprises the sequence of SEQ ID NO: 165, where the fusion protein is comprised of BCMA operatively linked to BCMA operatively linked to BCMA operatively linked to a Fc/IgG4 region operatively linked to BAFFR operatively linked to BAFFR operatively linked to BAFFR.

As another example, in some embodiments, an exemplary fusion protein is provided in which the fusion protein comprises the sequence of SEQ ID NO: 166, where the fusion protein is comprised of BCMA operatively linked to BCMA operatively linked to a Fc/IgG1 region operatively linked to BAFFR operatively linked to BAFFR operatively linked to BAFFR.

As another example, in some embodiments, an exemplary fusion protein is provided in which the fusion protein comprises the sequence of SEQ ID NO: 167, where the fusion protein is comprised of CD40 operatively linked to a Fc region operatively linked to BAFFR.

As another example, in some embodiments, an exemplary fusion protein is provided in which the fusion protein comprises the sequence of SEQ ID NO: 168, where the fusion protein is comprised of CD40 operatively linked to a Fc region operatively linked to BAFFR operatively linked to BAFFR.

As another example, in some embodiments, an exemplary fusion protein is provided in which the fusion protein comprises the sequence of SEQ ID NO: 169, where the fusion protein is comprised of CD40 operatively linked to a Fc region operatively linked to BAFFR operatively linked to BAFFR operatively linked to BAFFR.

As another example, in some embodiments, an exemplary fusion protein is provided in which the fusion protein comprises the sequence of SEQ ID NO: 170, where the fusion protein is comprised of CD40 operatively linked to a Fc region operatively linked to BAFFR operatively linked to BCMA.

As another example, in some embodiments, an exemplary fusion protein is provided in which the fusion protein comprises the sequence of SEQ ID NO: 171, where the fusion protein is comprised of CD40 operatively linked to a Fc region operatively linked to BCMA.

As another example, in some embodiments, an exemplary fusion protein is provided in which the fusion protein comprises the sequence of SEQ ID NO: 172, where the fusion protein is comprised of CD40 operatively linked to a Fc region operatively linked to BCMA operatively linked to BCMA.

As another example, in some embodiments, an exemplary fusion protein is provided in which the fusion protein comprises the sequence of SEQ ID NO: 173, where the fusion protein is comprised of CD40 operatively linked to a Fc region operatively linked to BCMA operatively linked to BCMA operatively linked to BCMA.

As another example, in some embodiments, an exemplary fusion protein is provided in which the fusion protein comprises the sequence of SEQ ID NO: 174, where the fusion protein is comprised of CD40 operatively linked to a Fc region operatively linked to BAFFR operatively linked to BCMA.

As another example, in some embodiments, an exemplary fusion protein is provided in which the fusion protein comprises the sequence of SEQ ID NO: 175, where the fusion protein is comprised of CD40 operatively linked to a Fc region operatively linked to BCMA.

As another example, in some embodiments, an exemplary fusion protein is provided in which the fusion protein comprises the sequence of SEQ ID NO: 176, where the fusion protein is comprised of CD40 operatively linked to a Fc region operatively linked to BCMA operatively linked to BCMA.

As another example, in some embodiments, an exemplary fusion protein is provided in which the fusion protein comprises the sequence of SEQ ID NO: 177, where the fusion protein is comprised of CD40 operatively linked to a Fc region operatively linked to BCMA operatively linked to BCMA operatively linked to BCMA.

As another example, in some embodiments, an exemplary fusion protein is provided in which the fusion protein comprises the sequence of SEQ ID NO: 178, where the fusion protein is comprised of common gamma chain operatively linked to IL21R operatively linked to a Fc region operatively linked to BAFFR.

As another example, in some embodiments, an exemplary fusion protein is provided in which the fusion protein comprises the sequence of SEQ ID NO: 179, where the fusion protein is comprised of IL23R operatively linked to IL12R beta-1 operatively linked to a Fc region operatively linked to BAFFR operatively linked to BAFFR.

As another example, in some embodiments, an exemplary fusion protein is provided in which the fusion protein comprises the sequence of SEQ ID NO: 180, where the fusion protein is comprised of an extracellular domain of immune checkpoint protein PD-1 r (Programmed Cell Death Protein 1) operatively linked to a Fc region operatively linked to a recombinant VEGF receptor protein. In some embodiments, such a fusion protein of SEQ ID NO: 180 is believed to be particularly useful in treating various cancers as the soluble PD-1 is capable of providing tumor suppressing mechanism via its PD-L1 and PD-L2 domains while the soluble VEGF receptor suppresses or otherwise reduces tumor angiogenesis.

As another example, in some embodiments, an exemplary fusion protein is provided in which the fusion protein comprises the sequence of SEQ ID NO: 181, where the fusion protein is comprised of an extracellular domain of BDCA2 operatively linked to a Fc region operatively linked to recombinant BAFFR.

As another example, in some embodiments, an exemplary fusion protein is provided in which the fusion protein comprises the sequence of SEQ ID NO: 182, where the fusion protein is comprised of an extracellular domain of BDCA2 operatively linked to a Fc region operatively linked to recombinant CD40.

With further regard to the exemplary fusion polypeptides described herein, in some embodiments, the fusion polypeptide comprises a sequence selected from the group consisting of SEQ ID NOS: 72-182, such as, in certain embodiments, a sequence selected from the group consisting of SEQ ID NOS: 102-119 or a sequence selected from the group consisting of SEQ ID NOS: 73-74 and 118-119. In some embodiments, the fusion polypeptide comprises the sequence of SEQ ID NO: 120.

In some embodiments of the presently-described fusion polypeptides, the fusion polypeptide comprises a sequence selected from the group consisting of SEQ ID NOS: 72-124, where the linker polypeptides included in those sequences can be interchanged for any of the linker peptides selected from the group consisting of SEQ ID NOS: 43-71.

In some embodiments of the fusion peptides, the ligand binding domain comprises a BAFFR selected from the group consisting of: SEQ ID NOS: 2-8. In certain of those BAFFR-including fusion polypeptides, the fusion protein comprises two or more BAFFR selected from the group consisting of: SEQ ID NOS: 3-8.

In some embodiments of the fusion polypeptides, the ligand binding domain comprises: an IL-17 Receptor having the sequence of SEQ ID NO: 1; an IL-21 Receptor selected from the group consisting of SEQ ID NOS: 16 and 24; a TNF Receptor selected from the group consisting of SEQ ID NOS: 10-12, 19, and 25; and/or an IFN Receptor selected from the group consisting of SEQ ID NOS: 9 and 14-15.

In some embodiments, the fusion polypeptides include an Fc region comprising an amino acid sequence selected from the group consisting of SEQ ID NOS: 26-42 or 26-36. In some embodiments, the Fc region has a sequence of SEQ ID NO: 29 or 30. In some embodiments, the Fc region has the sequence of SEQ ID NO: 37.

In some embodiments, it is further contemplated that an exemplary IL-17AR ligand binding domain used in accordance with the presently-disclosed fusion peptides can be replaced with a ligand binding domain selected from one or more of: IL-17BR, IL-17CR, IL-17DR, IL-17ER, IL17FR, TNFR1, TNFR2, TNFR3, TNFR4, TNFR5 or TNFR5. It is also contemplated that in certain embodiments a soluble receptor domain in any number of the aforementioned embodiments may be replaced with a ligand binding domain of the cytokine receptor subunit gamma, CD-40 CTLA-4, ICOS-R, CD27 antigen, or CD38 antigen.

In some further embodiments of the fusion polypeptides of the presently-disclosed subject matter are fusion polypeptides that includes at least two ligand binding domains where at least on ligand binding domain inhibits B cell activity and at least one ligand binding domain inhibits T cell activity. In some such embodiments, the at least one ligand binding domain that inhibits B cell activity and/or the at least one ligand binding domain that inhibits T cell activity slows the progression of Sjogren's Syndrome.

In some embodiments of the presently-disclosed subject matter, isolated nucleic acids are further provided that comprise a nucleotide sequence encoding a fusion polypeptide of the present disclosed subject matter. In some embodiments, an isolated nucleic acid is provided encoding the fusion protein of the presently-discloses subject matter, such as, in certain embodiments, a fusion protein having the amino acid sequence of SEQ ID NOS: 72-182 or fragments or variants thereof. In some embodiments, the isolated fusion proteins or isolated nucleic acids described herein have sequences that are about 50%, about 60%, about 70%, about 80%, about 90%, or more homologous to the described amino acid or nucleic acid sequences. “Percent identity,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). To obtain gapped alignments for comparison purposes, Gapped BLAST is utilized as described in Altschul, et al. (Nucleic Acids Res. 25: 3389-3402, 1997). When utilizing BLAST and Gapped BLAST programs, the default parameters of the respective programs (e.g., XBLAST and NBLAST) are used. Moreover, percent homology of sequences can be determined using the most recent version of BLAST, as of the filing date of this application.

To generate an exemplary fusion polypeptide in accordance with the presently-disclosed subject matter, in some embodiments, the nucleic acid encoding the fusion polypeptide is inserted into an appropriate expression vector that contains the necessary elements for the transcription and translation of the inserted protein-coding sequence. A variety of host-vector systems can be utilized to express an inserted protein-coding sequence, including mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements can be used. As one exemplary embodiment of a vector comprising a nucleic acid sequence of the presently disclosed subject matter, an exemplary vector can be a plasmid, such a plasmid into which a nucleic acid encoding an fusion polypeptide can be cloned by the use of internal restriction sites present within the vector.

In some embodiments, the nucleic acids of the presently-disclosed subject matter are operably linked to an expression cassette. The terms “associated with”, “operably linked”, and “operatively linked,” when used herein in reference to a nucleic acid sequence, refer to two nucleic acid sequences that are related physically or functionally. For example, a promoter or regulatory DNA sequence is said to be “associated with” a DNA sequence that encodes an RNA or a polypeptide if the two sequences are operatively linked, or situated such that the regulator DNA sequence will affect the expression level of the coding or structural DNA sequence.

The term “expression cassette” refers to a nucleic acid molecule capable of directing expression of a particular nucleotide sequence in an appropriate host cell, and comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression.

Once generated by an appropriate host-vector system, the fusion polypeptides can then be separated and purified by an appropriate combination of known techniques. These methods include, for example: methods utilizing solubility such as salt precipitation and solvent precipitation; methods utilizing the difference in molecular weight, such as dialysis, ultra-filtration, gel-filtration, and SDS-polyacrylamide gel electrophoresis; methods utilizing a difference in electrical charge, such as ion-exchange column chromatography; methods utilizing specific affinity, such as affinity chromatography; methods utilizing a difference in hydrophobicity, such as reverse-phase high performance liquid chromatography; methods utilizing a difference in isoelectric point, such as isoelectric focusing electrophoresis; and, metal affinity columns, such as Ni-NTA. If an operatively linked purification tag, such as HA, is included in the fusion polypeptide, the purification tag can be utilized to purify the fusion polypeptide.

Further, polyclonal antibodies, monoclonal antibodies (mAb), antibody fragments, and receptor-Fc fusions are generally purified by affinity chromatography. Resins containing an immobilized ligand (e.g., protein A, protein G or protein L) are used to capture antibodies and antibody fragments. Additional purification steps for antibodies or fragments can include Ion-exchange (IEX) chromatography and Gel filtration (GF) A. Hydrophobic interaction (HIC) chromatography can also be used as a polishing step.

In some embodiments, an isolated cell is provided that comprises a nucleotide sequence that encodes a fusion polypeptide comprised of at least two ligand binding domains and a Fc region in accordance with the presently-disclosed subject matter. Nucleic acids containing a target nucleotide sequence (e.g., a nucleotide sequence encoding a fusion polypeptide of the presently-disclosed subject matter) operably linked to a regulatory sequence can be introduced into a host cell transiently or, for long term regulation of gene expression, the nucleic acid can be stably integrated into the genome of the host cell or remain as a stable episome in the host cell.

As used herein, the term “host cell” is intended to include any cell or cell line, including prokaryotic and eukaryotic cells, into which a nucleic acid sequence of the presently-disclosed subject matter can be introduced and expressed. Exemplary host cells include, but are not limited to, yeast, fly, worm, plant, frog, and mammalian cells. Non-limiting examples of mammalian cell lines which can be used include CHO-cells (Urlaub and Chasm (1980) Proc. Natl. Acad. Sci. USA, 77:4216-4220), 293 cells (Graham et al. (1977) J Gen. Virol., 36:59) or myeloma cells like SP2 or NSO (Galfre and Milstein (1981) Meth. Enzymol., 73(B):3-46). Other exemplary eukaryotic host cells include insect (e.g., Sp. frugiperda), yeast (e.g., S. cerevisiae, S. pombe, P. pastoris, K. lactis, H. polymorpha; as generally reviewed by Fleer, R. (1992) Current Opinion in Biotechnology, 3(5):486496)), fungal and plant cells. Specific exemplary prokaryotic host cells include E. coli and Bacillus sp.

Nucleic acids comprising a nucleotide sequence of the presently-disclosed subject matter operably linked to a regulatory sequence can be introduced into a host cell by standard techniques for transfecting cells. As used herein, the term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook, et al. (Molecular Cloning: A Laboratory Manual, 2nd Edition, Cold Spring Harbor Laboratory press (1989)), and other laboratory manuals.

Nucleotide sequences of the presently-disclosed subject matter operably linked to a regulatory sequence can be introduced into cells growing in culture by conventional transfection techniques (e.g., calcium phosphate precipitation, DEAE-dextran transfection, electroporation, lipids, etc.). In some embodiments, nucleotide sequences can also be transferred into cells in vivo, for example, by application of a delivery mechanism suitable for introduction of nucleic acid into cells in vivo into host production animals, such as retroviral vectors (see, e.g., Ferry, N. et al. (1991) Proc. Natl. Acad. Sci. USA, 88:8377-8381; and Kay, M. A. et al. (1992) Human Gene Therapy, 3:641-647), adenoviral vectors (see e.g., Rosenfeld, M. A. (1992) Cell, 68:143-155; and Herz, J. and Gerard, R D. (1993) Proc. Natl. Acad. Sci. USA, 90:2812-2816), receptor-mediated DNA uptake (see e.g., Wu, G. and Wu, C. H. (1988) J. Biol. Chem., 263:14621; Wilson et al. (1992) J Biol. Chem., 267:963-967; and U.S. Pat. No. 5,166,320), direct injection of DNA (see e.g., Acsadi et al. (1991) Nature, 332:815-818; and Wolff et al. (1990) Science, 247:1465-1468) or particle bombardment (see e.g., Cheng, L. et al. (1993) Proc. Natl. Acad. Sci. USA, 90:4455-4459; and Zelenin, A. V. et al. (1993) FEBS Letters, 315:29-32).

With further regard to the production of the fusion polypeptides of the presently-disclosed subject matter, although the above-described fusion peptides have been described with reference to a single polypeptide including two or more ligand binding domain and an Fc region, the presently-disclosed fusion polypeptides are further inclusive of multi-receptor polypeptides that can have heterodimeric fusion regions. It is appreciated that many receptors are composed of two different subunits, such as the receptors for IL-1α, IL-1β, IL-4, IL-12, IL-21, IL-23, IL-27, IL-33, IL-36, IL-37, Interferon alpha/beta, and others, while some of the cytokine families such as IL-36 have several members. It is further appreciated that a typical agonist signaling is initiated by a cytokine, such as IL-1β, binding its cognate receptor IL-1RI with nM affinity. Upon binding, a shared co-receptor, IL-1RAcP, is then recruited by binding to the composite surface of the cytokine and primary receptor complex, resulting in the creation of a ternary complex. The binding affinity of IL-1RAcP is approximately 100-fold weaker than that of the IL-1β/IL-1RI complex. As the trimeric complex containing the cytokine, primary receptor, and accessory protein is formed, the cytoplasmic TIR domains of the two receptors are brought together to elicit downstream signaling. Similarly, it is further appreciated that in the IL-2, IL-3, and IL-6 subfamilies, a subunit is commonly shared among the receptors members (e.g. gp130 for the Il-6 subfamily), and it is appreciated that at least six interleukin receptors share the common gamma chain. Likewise, an interleukin receptor has been observed which binds interleukin-12 with low affinity and is involved in IL12 transduction, where once associated with IL12RB2, it forms a functional, high affinity receptor for IL-12, and where it also associated with IL23R to form the interleukin-23 receptor which functions in IL-23 signal transduction probably through activation of the Jak-Stat signaling cascade.

To produce such heterodimeric proteins, in some embodiments, the receptor subunits can be cloned in line behind one promoter (with our without a peptide linker to facilitate folding). The two genes can then be cloned in front or behind the desired Fc molecule or another carrier protein such a HSA or Transferrin. Similar to bispecific antibodies, the two genes encoding the ligand binding domains (e.g., the receptor subunits) can then be expressed using two separated transcriptional units in front of or behind Fc molecules to obtain two transcriptional units: Promoter-Receptor1-Fc and Promoter-Receptor2-Fe. Due to the Fc region, the two proteins can then dimerize via disulfide bridges.

In some embodiments, in order to form the desired heterodimers and to avoid homodimer formation, the Fc part can be engineered in various ways. For example, in some embodiments, the KiH (Knob in Hole) concept can be utilized with the use of a variety of well-characterized mutations (e.g., T366Y (knob), Y407T (hole)). Complementing these mutations with F405A and T394 on the knob and hole sites, respectively, increases heterodimer formation. Other useful mutations in that respect include H435R and Y436F, P228S and F405L and F405L/K409R. Additionally, in some embodiments, other concepts have also been proved to form more than 90% of heterodimers, e.g. by electrostatic optimizations, with useful mutations including K409, K360E and D399V, F405T, Q347R, E357N, S364B.

Further provided, in some embodiments of the presently-disclosed subject matter and rather than fusion proteins comprised of two or more ligand binding domains from soluble receptors, are fusion proteins that include ligand binding domains fused to the C-terminus of antibodies. For example, it is appreciated that the BAFF-receptor (BAFFR) is encoded by the TNFRSF13C gene and is one of the pro-survival receptors in B cells. Its expression starts when the immature B cells develop to transitional B cells, which then receive BAFFR-dependent pro-survival signals to rescue them from premature cell death. The BAFFR ligand (BAFF/BLyS) prolongs the survival of B cells, stimulates maturation, and promotes survival of autoreactive B cells. Excessive BAFF production leads to autoimmunity, presumably as the consequence of inappropriate survival of self-reactive B cells. In this way, a fusion protein including an antibody targeting CD 20 (e.g., Rituximab) fused to the BAFFR or several BAFFRs targets not only mature B cells but also early pro-B cells.

As another example, it is appreciated that Secukinumab is an IgG1κ monoclonal antibody that binds and inhibits the protein interleukin-17A. The mAB is used to treat of psoriasis, ankylosing spondylitis, and psoriatic arthritis In some embodiments, two soluble BAFFRs are thus fused to the C-terminus of Secukinumab (COSENTYX) (e.g., using linker peptides 15 amino acids in length) and can be produced having the amino acid sequence of SEQ ID NOS: 183-184. In a further embodiment, three soluble BCMA receptors fused to the C-terminus of Secukinumab using linkers that are 10 amino acids in length as provided in SEQ ID NOS: 185-186.

As another example, in some embodiments, a fusion polypeptide is provided in which a soluble TACI and a soluble BAFF receptor are fused to the C-terminus of the heavy chain of Adalimumab using a 23 amino acid linker peptide and a 5 amino acid linker peptide between TACI and BAFFR as shown and provided in SEQ ID NO: 187-188.

As another example, in some embodiments, Pemobrolizumab, which is useful for the treatment of melanoma, lung cancer, head and neck cancer, Hodgkin lymphoma, stomach cancer, cervical cancer, and certain types of breast cancer can also be incorporated into a fusion peptide of the presently-disclosed subject matter. In some embodiments, a fusion polypeptide is provided in which a soluble recombinant VEGF receptor is fused to Pemobrolizumab as shown and provided in SEQ ID NOS: 189-190.

In some further embodiments of the present invention that make use of antibody and soluble receptor combinations, soluble receptors can also be fused to bispecific antibodies resulting in therapeutics targeting 3 or more ligands (e.g., cytokines, receptors, etc.). For example, Amivantamab (Rybrevant) is a bispecific antibody directed against epidermal growth factor (EGF) and MET receptors and can be used to treat certain non-small cell lung cancers (NSCLC). In such a bispecific antibody, the soluble receptor(s) is (are) fused to the C-terminus of the two engineered heavy chains (A-chain and B-chain) such that adding one soluble receptor creates a molecule for 3 targets (Triple Trap) and such that adding 2 different receptors results in a Quadruple Trap. In some such embodiments, the first Target can be the Met-receptor, the second target can be EGF, and the third target can be VEGF A.

In some other embodiments that make use of a bi-specific antibody fusion, a fusion protein is provided that comprises a bispecific CD20/CD3 antibody operatively linked to a BAFFR operatively linked to a BAFF, such as that provided in SEQ ID NOS: 191-194. In some embodiments, the light chains for use with such bispecific CD20/CD3 antibodies, can be provided in a separate expression cassette.

In a further embodiment of the fusion proteins described herein that make use of antibodies fused to one or more ligand binding domains, an exemplary fusion protein is provided in which the fusion protein comprises the sequences of SEQ ID NOS: 195-196, which are comprised of an anti-IFN type 1 receptor antibody operatively linked to BAFFR operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequence of SEQ ID NOS: 197-198, which are comprised of RITUXIMAB (ANTI CD20) operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequences of SEQ ID NOS: 199-200, which are comprised of SARILUMAZB (ANTI IL-6R ALPHA) operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequences of SEQ ID NO: 200-201, which are comprised of TAFASITAMAB (ANTI-CD19) operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequences of SEQ ID NOS: 203-204, which are comprised of BELIMUMAB operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequences of SEQ ID NO: 205-206, which are comprised of RITUXIMAB (ANTI CD20) operatively linked to BAFFR.

In another embodiment of the fusion proteins described herein that make use of antibodies fused to one or more ligand binding domains, an exemplary fusion protein is provided in which the fusion protein comprises the sequences of SEQ ID NO: 207-208, which are comprised of RITUXIMAB (ANTI CD20) operatively linked to BAFFR operatively linked to BAFFR operatively linked to BAFFR.

In another embodiment of the fusion proteins described herein that make use of antibodies fused to one or more ligand binding domains, an exemplary fusion protein is provided in which the fusion protein comprises the sequences of SEQ ID NO: 209-210, which are comprised of RITUXIMAB (ANTI CD20) operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 211-212, which are comprised of RITUXIMAB (ANTI CD20) operatively linked to TACI operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 213-214, which are comprised of RITUXIMAB (ANTI CD20) operatively linked to TACI operatively linked to TACI operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 215-216, which are comprised of RITUXIMAB (ANTI CD20) operatively linked to BCMA.

In another embodiment of the fusion proteins described herein that make use of antibodies fused to one or more ligand binding domains, an exemplary fusion protein is provided in which the fusion protein comprises the sequences of SEQ ID NOS: 217-218, which are comprised of RITUXIMAB (ANTI CD20) operatively linked to BCMA operatively linked to BCMA.

In another embodiment of the fusion proteins described herein that make use of antibodies fused to one or more ligand binding domains, an exemplary fusion protein is provided in which the fusion protein comprises the sequences of SEQ ID NOS: 219-220, which are comprised of RITUXIMAB (ANTI CD20) operatively linked to BCMA operatively linked to BCMA operatively linked to BCMA.

In another embodiment of the fusion proteins described herein that make use of antibodies fused to one or more ligand binding domains, an exemplary fusion protein is provided in which the fusion protein comprises the sequences of SEQ ID NO: 221-222, which are comprised of RITUXIMAB (ANTI CD20) operatively linked to BAFFR operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 223-224, which are comprised of Belimumab operatively linked to BAFFR.

In another embodiment of the fusion proteins described herein that make use of antibodies fused to one or more ligand binding domains, an exemplary fusion protein is provided in which the fusion protein comprises the sequences of SEQ ID NOS: 225-226, which are comprised of Belimumab operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 227-228, which are comprised of Belimumab operatively linked to BCMA.

In another embodiment of the fusion proteins described herein that make use of antibodies fused to one or more ligand binding domains, an exemplary fusion protein is provided in which the fusion protein comprises the sequences of SEQ ID NOS: 229-230, which are comprised of Belimumab operatively linked to BAFFR operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 231-232, which are comprised of Secukinumab operatively linked to soluble receptor IL-17RC.

In another embodiment of the fusion proteins described herein that make use of antibodies fused to one or more ligand binding domains, an exemplary fusion protein is provided in which the fusion protein comprises the sequences of SEQ ID NOS: 233-234, which are comprised of Teplizumab operatively linked to TACI.

In another embodiment of the fusion proteins described herein that make use of antibodies fused to one or more ligand binding domains, an exemplary fusion protein is provided in which the fusion protein comprises the sequences of SEQ ID NOS: 235-236, which are comprised of Lecanemab operatively linked to IL-10.

In further embodiments of the present invention, additional exemplary, receptor-antibody domain combinations include: BR3-Fc-IFNaRAb (making use of the binding domain sequence from anifrolumab); BR3-Fc-IL17a Ab (binding sequence of secukinumab); BR3-Fc-TNFaAB (binding sequence of Humira); BR3-Fc-CD20 Ab (binding sequence of Rituxan); BR3-Fc-CD38 Ab; mAB-CD20-sPD1 (with improved Fc or afucosylated); mAb CD20-Fc-BAFFR; mAb CD20-Fc-BAFFR-BAFFR; mAb CD20-Fc-BAFFr-BAFFR-BAFFR; mAB CD20-FcTACI; mAb CD20-Fc-BAFFR-TACI; mAB CD20-Fc-BAFFR-BCMA; mAB CD38 (same constructs as above); mAb PD1-VEGFR; mAb PD1-CTLA4R; mAB SPD1-FC; mAB sPD1-Fc-VEGFR; mAB sPD1-Fc-BAFFR(1-3) (B cell lymphoma); mAB sPD1-Fc-TACI (1-3); mAB sPD1-Fc-BAFFR (2)-TACI (2); mAB sPD1-Fc-BAFFR-VEGFR; mAB PD1R-Fc VEGFR; and mAB PD1R-Fc-CTLA4R.

In some embodiments of the presently-disclosed subject matter, fusion proteins are provided that make use of an antibody binding regions in connection with ligand binding domains where the antibody regions comprise Fc regions which differ in composition such that the molecules form heterodimers. Of course, it will be appreciated by those skilled in the art that the exemplary fusion proteins described herein that include and make use of antibody regions consisting of heavy and light chains and Fc regions as well as ligand binding domains (e.g., from soluble receptors) can be constructed and expressed as separate gene products and/or can be combined as gene products that are included in the same expression cassette.

In some embodiments, one or more additional fusion proteins are provided including or otherwise making use of one or more of the features or domains of the exemplary fusion proteins described above. For example, in some embodiments, a fusion protein is provided comprising a soluble receptor or ligand binding domain operatively connected to an Fc region and a cytokine. In some embodiments, as one example of such a fusion proteins, an exemplary fusion protein is provided in which the fusion protein comprises the sequence of SEQ ID NO: 237, where the fusion protein is comprised of IL-2 operatively linked to a Fc region operatively linked to BAFFR.

As another example, in some embodiments, an exemplary fusion protein is provided in which the fusion protein comprises the sequence of SEQ ID NO: 238, where the fusion protein is comprised of IL-2 operatively linked to a Fc region operatively linked to BAFFR operatively linked to BAFFR.

As another example, in some embodiments, an exemplary fusion protein is provided in which the fusion protein comprises the sequence of SEQ ID NO: 239, where the fusion protein is comprised of IL-10 operatively linked to a Fc region operatively linked to TNFR2.

As another example, in some embodiments, an exemplary fusion protein is provided in which the fusion protein comprises the sequence of SEQ ID NO: 240, where the fusion protein is comprised of IL-10 operatively linked to a Fc region operatively linked to IL17RA.

As another example, in some embodiments, an exemplary fusion protein is provided in which the fusion protein comprises the sequence of SEQ ID NO: 241, where the fusion protein is comprised of IL-10 operatively linked to a Fc region operatively linked to BAFFR operatively linked to BAFFR.

As another example, in some embodiments, an exemplary fusion protein is provided in which the fusion protein comprises the sequence of SEQ ID NO: 242, where the fusion protein is comprised of IL-10 operatively linked to a Fc region operatively linked to I1-2.

As another example, in some embodiments, an exemplary fusion protein is provided in which the fusion protein comprises the sequence of SEQ ID NO: 243, where the fusion protein is comprised of IL-17RA operatively linked to an Albumin Binding Site operatively linked to BAFFR.

As another example, in some embodiments, an exemplary fusion protein is provided in which the fusion protein comprises the sequence of SEQ ID NO: 244, where the fusion protein is comprised of CD40 operatively linked to an Albumin Binding Site operatively linked to BAFFR operatively linked to BAFFR.

With further regard to the fusion proteins comprising soluble receptor, Fc regions, or monoclonal antibodies that can be fused to cytokines, it is appreciated that anti-inflammatory cytokines are of special interest for number of autoimmune diseases, and include inflammatory cytokines such as IL-4, IL-10, IL-11, and IL-13. Indeed, IL-10 is recognized as an immune regulatory cytokine that acts on many cells of the immune system, where it has a profound anti-inflammatory function, by, at least in part, limiting excessive tissue disruption caused by inflammation. Mechanistically, IL10 binds to its heterotetrameric receptor comprising IL10RA and IL10RB, which then leads to JAK1 and STAT2-mediated phosphorylation of STAT3. STAT3 then translocates to the nucleus where it drives expression of anti-inflammatory mediators. IL-10 also inhibits the production of chemokines that are involved in inflammation and has inhibitory effects on the production of reactive oxygen species (ROS) and nitric oxide (NO). However, IL-10 not only inhibits the production of proinflammatory molecules, but IL-10 also induces the production of anti-inflammatory molecules, such as the IL-1 receptor antagonist (IL-IRA) and soluble TNF receptors. For additional information and guidance regarding the use of IL-10, see, e.g., International Patent Application Publication No. WO2012045334, which is incorporated herein by reference in its entirety.

In some embodiments, rather making use of IL-10 in an exemplary fusion proteins, it is contemplated that an IL-10 homologue (vIL-10) from Epstein Barr Virus (EBV) can also be used as it shares 83% amino acid sequence identity with hIL-10 and as functional studies have demonstrated that ebvIL-10 also suppresses proinflammatory cytokines. In some embodiments, more than one cytokine can be included in an exemplary fusion protein and protease cleavage sites and linker or spacers sequences can be introduced between the Fc sequence and/or between the cytokines. In other embodiments, rather than or in addition to making use of IL-10 in an exemplary fusion protein, IL-1ra, and IL-4 can be included. In some embodiments, the fusion protein includes IL-1ra, a secreted anti-inflammatory cytokine, which competes with active IL-1 and blocks binding to their common activating receptor, IL-1R1.

In yet further embodiments of the presently-disclosed fusion polypeptides, fusion proteins are provided that include or otherwise make use of a bispecific antibody with a cytokine. In some embodiments, a fusion protein is provided comprising a the heavy chains of teclistamab with human IL-2. In some embodiments, as one example of such a fusion proteins, an exemplary fusion protein is provided in which the fusion protein comprises the sequences of SEQ ID NOS: 245-248.

As another example of a bispecific antibody fused to a cytokine in accordance with the presently-disclosed subject matter, in some embodiments, a fusion protein is provided where the fusion protein is comprised of both heavy chains of teclistamab and human interferon gamma and where the fusion protein comprises the sequences of SEQ ID NOS: 249-252.

Still further provided, in some embodiments of the presently-disclosed subject matter, are pharmaceutical compositions that include the fusion proteins (e.g., the ligand binding domains connected to the Fc regions) described herein and a pharmaceutically-acceptable vehicle, carrier, or excipient.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid, and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption. Injectable depot forms are made by forming microencapsule matrices of the compositions in biodegradable polymers such as polylactide-polyglycolide, poly(orthoesters) and poly(anhydrides). Depending upon the ratio of compositions to biodegradable polymer and the nature of the particular biodegradable polymer employed, the rate of compositions release can be controlled. Depot injectable formulations can also be prepared by entrapping the compositions in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain surfactants such as triton, antioxidants, buffers, sugars such as sucrose, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt). The compositions can also be formulated in rectal compositions, creams or lotions, or transdermal patches.

Still further provided, in some embodiments of the presently-disclosed subject matter, are methods for treating Sjögren's Syndrome and Systemic Lupus Erythematosus. In some embodiments, a method for treating Sjögren's Syndrome and Systemic Lupus Erythematosus provided that comprises administering to a subject in need thereof a fusion polypeptide including at least two ligand binding domains, each ligand binding domain having an amino acid sequence selected from the group consisting of SEQ ID NOS.1-25 or a fragment or variant thereof, and a Fc region of IgG or a fragment or variant thereof.

The terms “treatment” or “treating,” as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; treatment directed to minimizing or partially or completely inhibiting the development of the associated disease, pathological condition, or disorder; and supportive treatment, that is, treatment employed to supplement another therapy directed toward the improvement of the associated disease, pathological condition, or disorder.

The terms “reducing,” “reduction,” “inhibiting,” “inhibition” and grammatical variations thereof do not necessarily refer to the ability to completely inactivate all target biological activity in all cases. Rather, the skilled artisan will understand that those terms refer to decreasing biological activity of a target, such as can occur when a ligand binds a site of the target, a protein in a biochemical pathway of the target is blocked, a non-native complexes with a target, or the like. Such decrease in biological activity can be determined relative to a control, wherein the control can be representative of an environment in which an inhibitor is not administered. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. In some embodiments, the increases and/or decreases described herein can be in reference to a control subject having Sjögren's Syndrome and Systemic Lupus Erythematosus and that has not been treated with one of the presently-disclosed fusion proteins. In other embodiments, the increases and/or decreases described herein can be in reference to a baseline obtained in a subject that is in need of treatment, but has not yet began a particular therapeutic regimen. In some embodiments, administration of the polypeptide antagonist in accordance with the presently-disclosed subject matter reduces or treats one or more of the underlying causes and/or symptoms of Sjögren's Syndrome and/or Systemic Lupus Erythematosus. For example, in some embodiments, administering the polypeptide antagonist increases a level of expression or activity of particular cytokine in the subject. Measurement of such reductions can be performed using routine procedures known to those of ordinary skill in the art.

For administration of a therapeutic composition as disclosed herein (e.g., a fusion protein), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the administration of the composition is via oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof.

Regardless of the route of administration, the compositions of the presently-disclosed subject matter are typically administered in amount effective to achieve the desired response. As such, the term “effective amount” is used herein to refer to an amount of the therapeutic composition (e.g., a fusion protein and a pharmaceutically vehicle, carrier, or excipient) sufficient to produce a measurable biological response (e.g., a decrease in Sjögren's Syndrome and/or Systemic Lupus Erythematosus or one of the symptoms thereof). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

With further regard to the therapeutic methods described herein, in some embodiments, the therapeutic methods make use of a fusion polypeptide, comprising a Fc region and at least two ligand binding domains, where one of the at least two ligand binding domains binds modulates B cell activity and the other of the at least two ligand binding domains modulates T cell activity. Such fusion proteins can be used, in certain embodiments, in a method of treating a subject having a mutation in an amino acid sequence or nucleic acid sequence encoding BAFF, whereby the subject is administered an effective amount of such a fusion protein. In some embodiments of these methods, wherein the subject have or otherwise exhibit a biomarker selected from: an ANA titer greater than 1:80; and anti-dsDNA greater than 30 IU/ml; an anti-Sm greater than 15 units/ml; a C3 less than 900 mg/liter; a C4 less than 60 mg/liter; and a CRP positive greater than 3 mg/ml. In some embodiments, the subject has or is at risk of developing Systemic Lupus Erythematosus flares (i.e., a measurable increase in disease activity in one or more organ systems and typically involving new or worse clinical signs and symptoms and/or lab measurements).

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Lastly, in some embodiments, it is further contemplated that the fusion polypeptides of the presently-disclosed subject matter can also be used for analytical and diagnostic purposes. Examples include such polypeptides being labelled with a detectable tag for in situ or in vitro binding studies to detect ligand concentrations.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. Some of the following examples are prophetic, notwithstanding the numerical values, results and/or data referred to and contained in the examples.

### EXAMPLES

### Example 1—Production and Analysis of Fusion Proteins

Exemplary soluble receptor fusion proteins are produced using fully human sequences, are affinity purified with protein A or protein G, and are observed to have the long half-lives of monoclonal antibodies. The fusion proteins are designed to exhibit high, medium, or no effector functions. Certain of the receptor fusion proteins are produced in mammalian cells such as CHO or NSO cell lines and are purified using affinity chromatography (Protein A).

Once produced, the binding affinity and stability of the soluble receptor fusion proteins to the target cytokines are measured as an initial step in characterizing the novel molecules. Ligand binding assays, such as ELISA (Enzyme-Linked Immunosorbent Assay), surface plasmon resonance (SPR), or functional assays, such as cell-based assays and flow cytometry, are also performed. Surface plasmon resonance (SPR) assays are further performed to determine a ligand's affinity and binding kinetics for its receptor as that technique measures the real-time binding association and dissociation rates.

For the cellular assays, many reporter cell lines are commercially available, and allow measurement of the signal transduction caused by cytokines. Experiments are undertaken to measure and quantify the inhibition provided by the soluble receptors of the fusion proteins added to the culture supernatants. Examples of such cell line-based assays include and make use of IL-17 reporter HEK 293 cells, HEK-Blue™ CD 40L reporter cells, HEK-Dual™ TNF-α cells, Type I IFN Reporter Cells, etc. Similar assays are available measuring the inhibition of VEGF and BAFF/BCMA. For IFN alpha, IFN gamma, and IL17, the same type of cell lines can be used to test and quantitate inhibition. One can use mAbs as controls. Confirmation and quantification of effector functions (ADCC, ADCP, Fc gamma RI,) are done next, before entering animal studies. Moreover, in a parallel effort, the growth inhibition caused by the receptor fusion proteins is studied in panels of cancer cell lines (breast, uterus, cervix, etc.)

Experiments are further performed to analyze the ability to increase the binding affinity and stability between soluble receptor of the fusion protein and its target protein. Random mutations are introduced into the binding domain of the receptor, or specific amino acids are changed within or close to the binding sites. The three-dimensional structures of most cytokine receptors and ligands have been determined, making the latter approach particularly useful. Creating these “hot spot” mutants reduces the number of mutants to be screened considerably. Identifying soluble receptors with increased binding activity are then isolated, such as by using phage display or yeast surface display technologies and panning. The combination with Illumina sequencing then gains vast sequence data and allows for a comprehensive analysis of the diversity and abundance of mutants and monitoring of the parameters during selection.

In the above-described binding and cellular assays, the controls include the binding domains included in SEQ ID NOS: 1-25 as well as ianalumab, dazodalibep, rituximab, benlysta.

### Example 2—Animal Studies

A number of animal models and scientific rationales are available for testing drugs in Sjogren's. Animal models for evaluation of potential efficacy of new therapies in Sjogren's disease are primarily evaluated in a variety of genetically modified murine inbred strains. There are also in vitro models and approaches using human and murine cell or tissue samples looking at specific aspects of disease pathogenesis, but those models do not represent a comprehensive Sjogren's disease model. Although there have been decades of research into pathogenesis of Sjogren's and animal models, there is not one model that mimics the majority of clinical signs and symptoms of human Sjogren's disease. Most murine animal models focus on exocrine gland (lacrimal gland-LG and salivary gland-SG) autoimmunity looking at immune cell infiltration leading to secretory dysfunction (Sicca syndrome dry eyes and mouth) and production of select autoantibodies. However, Sjogren's disease can affect nearly all organ systems. Murine models evaluate only a few or no other organ domains affected in human disease beyond Sicca syndrome. Some murine models have extra glandular manifestations, but the manifestations are usually due to genetic mutations identified that have arisen spontaneously or by transgenic or knockout mice, and often do not have glandular symptoms or the models lack Sjogren's associated autoantibodies (Ro/SSA, La/SSB, RF, ANA, M3R and CAII).

The murine Sjogren's models (see Park et al., Mouse Models of Primary Sjogren's, Curr. Pharm Des. 2016 and Gao et al. Recent Advances in Mouse Models of Sjogren's Syndrome, Frontiers in Immunol. 2020, the contents of both of which are incorporated herein by reference) consist of: 1) spontaneous genetic mutations identified in inbred mice with the most common being NOD mice used in diabetes or NOD derivatives changing MHC H-2 domain to H-2b background (NOD.B10-H2b) or using C57BL6 NOD Aec1Aec2 (contain two autoimmune exocrinopathy loci from NOD mice), where the change in background MHC eliminated onset of diabetes but retained the exocrine and autoimmune changes associated with Sjogren's; 2) NOD derivatives animal models that are slow to develop disease activity and either have no secretory dysfunction and no or limited Sjogren's related autoantibodies (Ro/SSA, La/SSB, RF, ANA, M3R and CAII); and 3) other spontaneous murine models: NFS/sld (requires neonatal thymectomy), IQI/Jic, CB57BL6 H-2b Aly/Aly. The first two models take 6-18 months for manifestations to evolve, while the latter two models do affect other organs including lungs, pancreas, kidney, or liver. All three models, however have limited secretory dysfunction and limited autoantibody production.

Other models include induced murine models, where immunization with submandibular gland homogenate in SL/nJ or CB57BL mice led to glandular infiltration and autoantibodies similar to Sjogren's. Enhanced Th17 response were observed and C57BL mice deficient in IL-17 challenged did not exhibit inflammatory exocrine autoimmune disease, supporting the importance of IL-17 in SS pathogenesis. Immunization of peptides from Ro60, CAII (carbonic anhydrase receptor) or M3R muscarinic receptor in different strains of mice led to infiltration with primarily T-lymphocytes (M3R) or both T and B-cells (Ro and CAII) into salivary and/or lacrimal glands with limited production of autoantibodies. CAII immunization led to involvement of pancreas and/or kidney.

Yet further models include viral induced exocrine inflammatory murine models by HTLV-1, MCMV and ADV5. Induction with these 3 viruses usually induces an inflammatory or autoimmune response in mouse strains prone to autoimmunity such as B6 or MRL/lpr mice. These models are not frequently used as host antiviral response and induction of autoimmunity may not be similar to Sjogren's without an infectious disease etiology,

In the MRL/lpr model, these mice are used mostly for testing in SLE models as they appear to have secondary Sjogren's with inflammatory infiltration of salivary and lacrimal glands with relevant Ro, La, ANA and RF autoantibodies. The most prominent feature is lupus nephritis that can be fatal as well as peripheral neuropathy. The model has responded to a variety of immunosuppressive drugs including anti-BAFF mAbs, and it has been appreciated that MRL/lpr mice present with glomerulonephritis, which is classic in SLE but rare (although not absent) in SS. Moreover, it is appreciated that the MRL/lpr mouse model has been identified as a model for neurological SLE and that MRL/lpr mice present with peripheral neuropathy, a rare complication of SLE but frequent in SS. Otherwise, the clinical, biological, and histological phenotype of MRL/lpr mice encompasses the key features of SS: female sex predilection, decreased salivary flow rate and tear production, lymphocytic infiltrates in the salivary and lacrimal glands, and anti-Ro/SSA and anti-La/SSB autoantibodies production. These mice also develop other manifestations resembling those of human patients with systemic SS, such as arthritis, pneumonitis, cryoglobulinemia, and signs of lymphoproliferation (lymphadenopathies, splenomegaly).

Another model to be utilized is a chimeric human-mouse model, where PBMCs from healthy volunteers or patients with SS are transferred into immunodeficient NOD-scid IL-2r) mouse recipients to produce chimeric mice. While no difference is observed in the distribution of cells, chimeric mice transferred with PBMCs from SjS patients produce enhanced cytokine levels. Histological examination reveal enhanced inflammatory responses in the lacrimal and salivary glands of SjS chimeras. Infiltrates are primarily CD4+, with minimal detection of CD8+ T-cells and B-cells. (see, e.g., Young et al. A Chimeric human-mouse model of Sjogren's Syndrome, J. Immunol. 2015, which is also incorporated herein by reference)..

In short, there are over 20 murine models of Sjogren's disease developed and tested in past several decades, but none of them mimic the majority Sjogren's disease manifestations, pathogenic mechanisms, autoantibodies and other biomarker (chemokine/cytokine changes) or increased risk for B-cell lymphomas. The NOD H-2b CB57BL mice is the most commonly used murine model for Sjogren's and is best at characterizing the exocrine glandular dysfunction. However, these mice do not manifest the more systemic and serious extra glandular aspects involving other organs like kidney, lungs, GI tract and musculoskeletal system. Other murine models of Sjogren's target pathogenic mechanisms due to selected genetic knockout or transgenic modifications.

What differentiates Sjogren's from other autoimmune diseases is exocrine inflammatory infiltration (B, T, and dendritic cells) into salivary and lacrimal glands leading to reduced secretion or Sicca syndrome of dry eyes and mouth. Therefore, one of the NOD H-2b models is used. The C57BL-6 NOD-Aec1/Aec2 model is to be first used as these mice manifest infiltration in LG and SG with secretory dysfunction in both glands and broad spectrum of Sjogren's specific autoantibodies. The model also has early onset of inflammatory manifestations at 8-10 weeks with continual progression of disease over 24 weeks. TACI-Fc in NOD mice leads to reduced inflammatory foci in SG due to decrease in IgD+ and CD 138+ lymphocytes, reduction of IgG, IgM, and inflammatory cytokines. Rapamycin effect on mTOR in NOD mice leads to suppressed lymphocytic infiltration in the LG. CD40 via AAV2-CD40:Fc in NOD mice; infiltrating cell types, Ig levels, SG output not reduced compared to control. IL-17 via Ad5-IL17R:Fc in C57BL-6 NOD-Aec1/Aec2 mice leads to a reduction of IL-17 levels leading to reduction of SG infiltration, improved saliva flow and normalization of ANA in both early and late stages of disease. CTLA-4 via AAV2-CTLA4IgG in C57BL-6 NOD-Aec1/Aec2 mice leads to blocked B7 expression on macrophages, improved secretion, decreased cellular infiltration and cytokine levels. No reduction of anti-Ro or anti-La autoantibodies. CXCL13 via anti-CXCL13 mAb 5378 in both NOD Id3 −/− and MRL/Mpj mice leads to improved SG inflammation, and has no effect on diabetes. Mucosal protective agent treatment with rebamipide in NFS/sld mice leads to improved saliva secretion, suppressed CD4+ T-cells and Th1 cytokines. Inhibition of autoantibodies, IgG, and IgM.

In addition to the above, ALPN-101 is a Fc fusion protein of a human inducible T cell costimulatory ligand (ICOSL) variant immunoglobulin domain (vIgDTM) designed to inhibit simultaneously the CD28 and ICOS costimulatory pathways. ALPN-101 treatment inhibited antibody production in vivo in mouse immunization models and suppressed proliferation and antibody production in human B cell/T cell co-cultures. In anti-PD-L1-treated NOD mice, ALPN-101 suppressed sialadenitis with activity superior to abatacept. Analyses of SjS-related autoantibodies were pending. Compared to abatacept, prezalumab, or combination abatacept+prezalumab, ALPN-101 demonstrated superior suppression of proinflammatory cytokine (i.e., TNF, IL-2, IL-6, IL-17A, GM-CSF, etc.) release from stimulated healthy or SjS patient PBMCs. Also, previously reported after CP-25 treatment, the lymphocyte infiltration in the salivary glands is reduced, and the salivary flow in NOD/Ltj experimental Sjogren's syndrome mice (ESS) is also improved. CP-25 MOA is probably associated with inhibiting the JAK1-STAT1/2-CXCL13 signaling pathway in SGECs, and this signaling pathway is related to the migration of B cells to salivary glands

In summary of the human therapeutic targets tested in Sjogren's Disease murine models, the potential pathogenic mechanisms for Sjogren's Disease are many and involve B-cells, T-cells, regulatory T-cells, and antigen presenting dendritic cells as well as many cytokines (including BAFF, IFN) and T-cell co-stimulatory factors including IL-17, CTLA-4, ICOSL, CD40. From a review of human clinical and murine models, some of the strongest associations with Sjogren's disease pathogenesis are BAFF, IL-17 and ICOSL. However, other T-cell co-stimulatory and co-inhibitory factors have been implicated and are logical to test in dual receptor therapies. Also, in reading other PD-L1 and PD-L2 co-inhibitory factors interacting with PD1 checkpoint inhibitor and TLRs have been implicated in Sjogren's disease pathogenesis as well as SLE. BCDA-2 receptor on dendritic cells has been implicated in autoimmunity as signaling interacts with TLR7/9. Monoclonal antibodies to BCDA-2 were shown to inhibit receptor signaling and be effective in ph2 SLE and CLE clinical trials.

There is a significant increased risk of developing a B-cell lymphoma over lifetime in patients 5-10% with Sjogren's disease. Although etiology or identification of these risk factors is not well defined, increased levels of BAFF have been implicated. As such, the ability to block BAFF by BAFFR dual receptor therapy such as by using the exemplary fusion proteins described herein are believed to be useful in preventing or slowing growth of B-cell lymphomas associated with Sjogren's. In addition, it is believed that in Sjogren's patients with high BAFF level or BAFF-var, the use of the presently-disclosed fusion proteins will confer a clinical benefit in reducing risk of B-cell lymphoma and possibly may slow already present B-cell lymphoma growth.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. American College Of Rheumatology, November 2022, Session: Abstracts: Sjögren's Syndrome—Basic And Clinical Science (1625-1628).
2. Arthritis & Rheumatism. Vol. 65, No. 8, August 2013, Pp 2143-2153, Arthritis & Rheumatism Vol. 64, No. 7, July 2012, Pp 2328-2337, N Engl J Med 2017; 376:1615-26.
3. Li P, Jin Y, Zhao R, Xue Z, Ji J. Expression Of ICOS In The Salivary Glands Of Patients With Primary Sjogren's Syndrome And Its Molecular Mechanism. Mol Med Rep. 2022 November; 26(5):348. Doi: 10.3892/Mmr.2022.12864. Epub 2022 Sep. 30. PMID: 36177915; PMCID: PMC9551409.
4. Bernhard Valldorf, Steffen C. Hinz, Giulio Russo, Lukas Pekar, Laura Mohr, Janina Klemm, Achim Doerner, Simon Krah, Michael Hust And Stefan Zielonka. Antibody Display Technologies: Selecting The Cream Of The Crop Biol. Chem. 2022; 403(5-6): 455-477
5. Karnell J. L. Et Al., 2019. Targeting The CD40-CD40L Pathway In Autoimmune Diseases: Humoral Immunity And Beyond. Adv Drug Deliv Rev. 141:92-103. Laman J. D. Et Al., 2017. Functions Of CD40 And Its Ligand, Gp39 (CD40L). Crit Rev Immunol. 37(2-6):371-420.
6. Elgueta R. Et Al., 2009. Molecular Mechanism And Function Of CD40/CD40L Engagement In The Immune System. Immunol. Rev. 229, 152-172.
7. Seijkens T. Et Al., 2013. CD40-CD40L: Linking Pancreatic, Adipose Tissue And Vascular Inflammation In Type 2 Diabetes And Its Complications. Diabetes And Vascular Disease Research. 10: 115-122.
8. Daoussis D. Et Al., 2004. Targeting CD40L: A Promising Therapeutic Approach. Clin Diagn Lab Immunol. 11(4):635-41.

It will be understood that various details of the presently disclosed subject matter can be changed without departing from the scope of the subject matter disclosed herein. Furthermore, the foregoing description is for the purpose of illustration only, and not for the purpose of limitation.

SEQUENCE LISTING
LIGAND BINDING SEQUENCES; EXTRACELLULAR DOMAINS
SEQ ID NO: 1 - AMINO ACID SEQUENCE OF EXTRACELLULAR DOMAIN
OF IL-17A RECEPTOR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
SEQ ID NO: 2 - AMINO ACID SEQUENCE OF BINDING DOMAIN OF BAFF
RECEPTOR
PTPCVPAECFDLLVRHCVACGLLRTPRPKP
SEQ ID NO: 3 - AMINO ACID SEQUENCE OF SOLUBLE BAFF RECEPTORS
MRRGPRSLRG RDAPAPTPCV PAECFDLLVR HCVACGLLRT PRPKPAGASS
PAPRTALQPQ ESVGAGAGEA ALPLPG
SEQ ID NO: 4 - AMINO ACID SEQUENCE OF SOLUBLE BAFF RECEPTORS
RRGPRSLRG RDAPAPTPCV PAECFDLLVR HCVACGLLRT PRPKPAGASS
PAPRTALQPQ ESVGAGAGEA
SEQ ID NO: 5 - AMINO ACID SEQUENCE OF SOLUBLE BAFF RECEPTORS
RGPRSLRG RDAPAPTPCV PAECFDLLVR HCVACGLLRT PRPKPAGASS PAPRTALQPQ
ES
SEQ ID NO: 6 - AMINO ACID SEQUENCE OF SOLUBLE BAFF RECEPTORS
DAPAPTPCV PAECFDLLVR HCVACGLLRT PRPKPAGASS PAPRTALQPQ
SEQ ID NO: 7 - AMINO ACID SEQUENCE OF SOLUBLE BAFF RECEPTORS
DAPAPTPCV PAECFDLLVR HCVACGLLRT PRPKPAGASS
SEQ ID NO: 8 - AMINO ACID SEQUENCE OF TWO BAFF RECEPTOR
LIGAND BINDING DOMAINS WITH FLEXIBLE LINKER
RGPRSLRG RDAPAPTPCV PAECFDLLVR HCVACGLLRT PRPKPAGASS PAPRTALQPQ
ESGGGGSGGGGSGGGGSRGPRSLRG RDAPAPTPCV PAECFDLLVR HCVACGLLRT
PRPKPAGASS PAPRTALQPQESG
SEQ ID NO: 9 - AMINO ACID SEQUENCE OF EXTRACELLULAR BINDING
DOMAIN OF IFNGR1
EMGTADLGPSSVPTPTNVTIESYNMNPIVYWEYQIMPQVPVFTVEVKNYGVKNSEWIDC
INISHHYCNISDHVGDPSNSLWVRVKARVGQKESAYAKSEEFAVCRDGKIGPPKLDIRK
EEKQIMIDIFHPSVFVNGDEQEVDYDPETTCYIRVYNVYVRMNGSEIQYKILTQKEDDCD
EIQCQLAIPVSSLNSQYCVSAEGVLHVWGVTTEKSKEVCITIFNSSIKG
SEQ ID NO: 10 - AMINO ACID SEQUENCE OF EXTRACELLULAR DOMAIN
OF TNFR-I
LVPHLGDREKRDSVCPQGKYIHPQNNSICCTKCHKGTYLYNDCPGPGQDTDCRECESGS
FTASENHLRHCLSCSKCRKEMGQVEISSCTVDRDTVCGCRKNQYRHYWSENLFQCFNC
SLCLNGTVHLSCQEKQNTVCTCHAGFFLRENECVSCSNCKKSLECTKLCLPQIENVKGT
EDSGTT
SEQ ID NO: 11 - AMINO ACID SEQUENCE OF EXTRACELLULAR DOMAIN
OF TNFR-2
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
SEQ ID NO: 12 - AMINO ACID SEQUENCE OF EXTRACELLULAR DOMAIN
OF TNFR-5
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
SEQ ID NO: 13 - AMINO ACID SEQUENCE OF EXTRACELLULAR DOMAIN
OF TACI RECEPTOR
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 14 - AMINO ACID SEQUENCE OF EXTRACELLULAR DOMAIN
OF IFNAR1
KNLKSPQKVEVDIIDDNFILRWNRSDESVGNVTFSFDYQKTGMDNWIKLSGCQNITSTK
CNFSSLKLNVYEEIKLRIRAEKENTSSWYEVDSFTPFRKAQIGPPEVHLEAEDKAIVIHIS
PGTKDSVMWALDGLSFTYSLVIWKNSSGVEERIENIYSRHKIYKLSPETTYCLKVKAALL
TSWKIGVYSPVHCIKTTVENELPPPENIEVSVQNQNYVLKWDYTYANMTFQVQWLHAF
LKRNPGNHLYKWKQIPDCENVKTTQCVFPQNVFQKGIYLLRVQASDGNNTSFWSEEIKF
DTEIQAFLLPPVFNIRSLSDSFHIYIGAPKQSGNTPVIQDYPLIYEIIFWENTSNAERKIIEK
KTDVTVPNLKPLTVYCVKARAHTMDEKLNKSSVFSDAVCEKTKPGNTSK
SEQ ID NO: 15 - AMINO ACID SEQUENCE OF EXTRACELLULAR DOMAIN
OF IFNAR2
ISYDSPDYTDESCTFKISLRNFRSILSWELKNHSIVPTHYTLLYTIMSKPEDLKVVKNCAN
TTRSFCDLTDEWRSTHEAYVTVLEGFSGNTTLFSCSHNFWLAIDMSFEPPEFEIVGFTNHI
NVMVKFPSIVEEELQFDLSLVIEEQSEGIVKKHKPEIKGNMSGNFTYIIDKLIPNTNYCVS
VYLEHSDEQAVIKSPLKCTLLPPGQESESAESAK
SEQ ID NO: 16 - AMINO ACID SEQUENCE OF EXTRACELLULAR DOMAIN
OF IL-21 RECEPTOR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
SEQ ID NO: 17 - AMINO ACID SEQUENCE OF EXTRACELLULAR DOMAIN
OF CYTOKINE RECEPTOR COMMON GAMMA CHAIN (IL2RG)
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
ATQMLKLQNLVIPWAPENLTLHKLSESQLELNWNNRFLNHCLEHLVQYRTDWDHSWT
EQSVDYRHKFSLPSVDGQKRYTFRVRSRFNPLCGSAQHWSEWSHPIHWGSNTSKENPFL
FALEA
SEQ ID NO: 18 - AMINO ACID SEQUENCE OF CYTOTOXIC T-
LYMPHOCYTE PROTEIN 4 (CTLA-4)
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
SEQ ID NO: 19 - AMINO ACID SEQUENCE OF TUMOR NECROSIS FACTOR
RECEPTOR SUPERFAMILY MEMBER 4 (CD134, OX40L RECEPTOR)
LHCVGDTYPSNDRCCHECRPGNGMVSRCSRSQNTVCRPCGPGFYNDVVSSKPCKPCTW
CNLRSGSERKQLCTATQDTVCRCRAGTQPLDSYKPGVDCAPCPPGHFSPGDNQACKPW
TNCTLAGKHTLQPASNSSDAICEDRDPPATQPQETQGPPARPITVQPTEAWPRTSQGPST
RPVEVPGGRA
SEQ ID NO: 20 - AMINO ACID SEQUENCE OF INDUCIBLE T-CELL
COSTIMULATORY RECEPTOR (ICOS-R)(CD278)
EINGSANYEMFIFHNGGVQILCKYPDIVQQFKMQLLKGGQILCDLTKTKGSGNTVSIKSL
KFCHSQLSNNSVSFFLYNLDHSHANYYFCNLSIFDPPPFKVTLTGGYLHIYESQLCCQLK
SEQ ID NO: 21 - AMINO ACID SEQUENCE OF CD27 ANTIGEN (RECEPTOR)
ATPAPKSCPERHYWAQGKLCCQMCEPGTFLVKDCDQHRKAAQCDPCIPGVSFSPDHHT
RPHCESCRHCNSGLLVRNCTITANAECACRNGWQCRDKECTECDPLPNPSLTARSSQAL
SPHPQPTHLPYVSEMLEARTAGHMQTLADFRQLPARTLSTHWPPQRSLCSSDFIR
SEQ ID NO: 22 - AMINO ACID SEQUENCE OF IL23R_HUMAN (UniProtKB -
Q5VWK5)
GITNINCSGHIWVEPATIFKMGMNISIYCQAAIKNCQPRKLHFYKNGIKERFQITRINKT
TARLWYKNFLEPHASMYCTAECPKHFQETLICGKDISSGYPPDIPDEVTCVIYEYSGNMT
CTWNAGKLTYIDTKYVVHVKSLETEEEQQYLTSSYINISTDSLQGGKKYLVWVQAANA
LGMEESKQLQIHLDDIVIPSAAVISRAETINATVPKTIIYWDSQTTIEKVSCEMRYKATTN
QTWNVKEFDTNFTYVQQSEFYLEPNIKYVFQVRCQETGKRYWQPWSSLFFHKTPETVP
QVTSKAFQHDTWNSGLTVASISTGHLTSDNRGDIG
SEQ ID NO: 23 - AMINO ACID SEQUENCE OF IL12RB1_HUMAN (UniProtKB -
P42701)
CRTSECCFQDPPYPDADSGSASGPRDLRCYRISSDRYECSWQYEGPTAGVSHFLRCCLSS
GRCCYFAAGSATRLQFSDQAGVSVLYTVTLWVESWARNQTEKSPEVTLQLYNSVKYEP
PLGDIKVSKLAGQLRMEWETPDNQVGAEVQFRHRTPSSPWKLGDCGPQDDDTESCLCP
LEMNVAQEFQLRRRQLGSQGSSWSKWSSPVCVPPENPPQPQVRFSVEQLGQDGRRRLT
LKEQPTQLELPEGCQGLAPGTEVTYRLQLHMLSCPCKAKATRTLHLGKMPYLSGAAYN
VAVISSNQFGPGLNQTWHIPADTHTEPVALNISVGTNGTTMYWPARAQSMTYCIEWQP
VGQDGGLATCSLTAPQDPDPAGMATYSWSRESGAMGQEKCYYITIFASAHPEKLTLWS
TVLSTYHFGGNASAAGTPHHVSVKNHSLDSVSVDWAPSLLSTCPGVLKEYVVRCRDED
SKQVSEHPVQPTETQVTLSGLRAGVAYTVQVRADTAWLRGVWSQPQRFSIEVQVSD
SEQ ID NO: 24 - AMINO ACID SEQUENCE OF IL21R_HUMAN (UniProtKB -
Q9HBE5)
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
SEQ ID NO: 25 - AMINO ACID SEQUENCE OF TUMOR NECROSIS FACTOR
RECEPTOR SUPERFAMILY MEMBER 9
LQDPCSNCPAGTFCDNNRNQICSPCPPNSFSSAGGQRTCDICRQCKGVFRTRKECSSTSN
AECDCTPGFHCLGAGCSMCEQDCKQGQELTKKGCKDCCFGTFNDQKRGICRPWTNCSL
DGKSVLVNGTKERDVVCGPSPADLSPGASSVTPPAPAREPGHSPQ
Fc SEQUENCES
SEQ ID NO: 26 - AMINO ACID SEQUENCE OF Fc IgG1 VARIANT (Etanercept
Long)
GSTGDEPKSC DKTHTCPPCP APELLGGPSV FLFPPKPKDT LMISRTPEVT
CVVVDVSHED PEVKFNWYVD GVEVHNAKTK PREEQYNSTY RVVSVLTVLH
QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL
TVDKSRWQQG NVFSCSVMHE ALHNHYTQKS LSLSPGK
SEQ ID NO: 27 - AMINO ACID SEQUENCE OF Fc IgG1 VARIANT (Alfibercept)
DKTHTCPPCP APELLGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
SEQ ID NO: 28 - AMINO ACID SEQUENCE OF Fc IgG1 VARIANT (M-V, N-S)
DKTHTCPPCP APELLGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVLHE ALHSHYTQKS LSLSPGK
SEQ ID NO: 29 - AMINO ACID SEQUENCE OF Fc IgG1 VARIANT (ALA,
Alfibercept)
DKTHTCPPCP APEAAGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
SEQ ID NO: 30 - AMINO ACID SEQUENCE OF Fc IgG1 VARIANT (N-A,
Alfibercept)
DKTHTCPPCP APELLGGPSV FLFPPKPKDT LMISRTPEVT CVVVDVSHED
PEVKFNWYVD GVEVHAAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
SEQ ID NO: 31 - AMINO ACID SEQUENCE OF Fc IgG1 VARIANT (S-D, I-E,
Alfibercept)
DKTHTCPPCP APELLGGPDV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PEEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
SEQ ID NO: 32 - AMINO ACID SEQUENCE OF Fc IgG1 VARIANT (M-L, N-S,
Alfibercept)
DKTHTCPPCPAPELLGGPSVFLFPPKPKDTLMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVLHE ALHSHYTQKS LSLSPGK
SEQ ID NO: 33 - AMINO ACID SEQUENCE OF Fc IgG1 VARIANT (Y-M, E-T,
E-D, M-L, Nirvesimab)
DKTHTCPPCPAPELLGGPSVF LFPPKPKDTL YITREPEVTC VVVDVSHEDP
EVKFNWYVDG VEVHNAKTKPREEQYNSTYR VVSVLTVLHQ DWLNGKEYKC
KVSNKALPAP IEKTISKAKG QPREPQVYTLPPSREEMTKN QVSLTCLVKG
FYPSDIAVEW ESNGQPENNY KTTPPVLDSD GSFFLYSKLTVDKSRWQQGN
VFSCSVMHEA LHNHYTQKSL SLSPGK
SEQ ID NO: 34 - AMINO ACID SEQUENCE OF Fc IgG1 VARIANT (LALA,
Etanercept, Delta)
DKTHTCPPCPAPEAAGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAKGQPREPQVYT LPPSREEMTK NQVSLTCLVK
GFYPSDIAVE WESNGQPENN YKTTPPVLDSDGSFFLYSKL TVDKSRWQQG
NVFSCSVMHE ALHNHYTQKS LSLSPGK
SEQ ID NO: 35 - AMINO ACID SEQUENCE OF Fc IgG1 VARIANT
(Margetuximab)
DKTHTCP PCPAPELVGGPSVFLLPPKP KDTLMISRTP EVTCVVVDVS HEDPEVKFNW
YVDGVEVHNA KTKPPEEQYNSTLRVVSVLT VLHQDWLNGK EYKCKVSNKA
LPAPIEKTIS KAKGQPREPQ VYTLPPSRDELTKNQVSLTC LVKGFYPSDI
AVEWESNGQP ENNYKTTPLV LDSDGSFFLY SKLTVDKSRWQQGNVFSCSV
MHEALHNHYT QKSLSLSPGK
SEQ ID NO: 36 - AMINO ACID SEQUENCE OF Fc IgG1 VARIANT (S-D,
Alfibercept)
DKTHTCPPCP APELLGGPDV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PEEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
SEQ ID NO: 37 - AMINO ACID SEQUENCE OF Fc IgG4 VARIANT
(Pembrolizumab)
KYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
SEQ ID NO: 38 - AMINO ACID SEQUENCE OF Fc IgG1 VARIANT (LALA,
Etanercept)
DKTHTCPPCP APEAAGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCMVSHE ALHNHYTQKS LSLSPGK
SEQ ID NO: 39 - AMINO ACID SEQUENCE OF Fc IgG2 VARIANT
(Crizanlizumab Long)
NVDHK PSNTKVDKTV ERKCCVECPP CPAPPVAGPS VFLFPPKPKD TLMISRTPEV
TCVVVDVSHE DPEVQFNWYV DGMEVHNAKT KPREEQFNST FRVVSVLTVV
HQDWLNGKEY KCAVSNKGLP APIEKTISKT KGQPREPQVY TLPPSREEMT
KNQVSLTCLV KGFYPSDIAV EWESNGQPEN NYKTTPPMLD SDGSFFLYSK
LTVDKSRWQQ GNVFSCSVMH EALHNHYTQK SLSLSPGK
SEQ ID NO: 40 - AMINO ACID SEQUENCE OF Fc IgG2 VARIANT
(Cosebilimab)
DKTHTCP PCPAPELLGGPSVFLFPPKP KDTLMISRTP EVTCVVVDVS HEDPEVKFNW
YVDGVEVHNA KTKPREEQYNSTYRVVSVLT VLHQDWLNGK EYKCKVSNKA
LPAPIEKTISKAKGQPREPQVYTLPPSRDELTKNQVSLTCLVKGFYPSDIAVEWESNGQP
ENNYKTTPPV LDSDGSFFLY SKLTVDKSRWQQGNVFSCSV MHEALHNHYT
QKSLSLSPGK
SEQ ID NO: 41 - AMINO ACID SEQUENCE OF Fc IgG4 VARIANT
(Pembrolizumab Long)
NVDHKPS NTKVDKRVES KYGPPCPPCP APEFLGGPSV
FLFPPKPKDT LMISRTPEVT CVVVDVSQED PEVQFNWYVD GVEVHNAKTK
PREEQFNSTY RVVSVLTVLH QDWLNGKEYK CKVSNKGLPS SIEKTISKAK
GQPREPQVYT LPPSQEEMTK NQVSLTCLVK GFYPSDIAVE WESNGQPENN
YKTTPPVLDS DGSFFLYSRL TVDKSRWQEG NVFSCSVMHE ALHNHYTQKS LSLSLGK
SEQ ID NO: 42 - AMINO ACID SEQUENCE OF Fc IgG1 VARIANT (Abatacept)
DKTH TSPPSPAPEL LGGSSVFLFP PKPKDTLMIS RTPEVTCVVV DVSHEDPEVK
FNWYVDGVEV HNAKTKPREE QYNSTYRVVS VLTVLHQDWL NGKEYKCKVS
NKALPAPIEK TISKAKGQPR EPQVYTLPPS RDELTKNQVS LTCLVKGFYP
SDIAVEWESN GQPENNYKTT PPVLDSDGSF FLYSKLTVDK SRWQQGNVFS
CSVMHEALHN HYTQKSLSLS PGK
LINKER SEQUENCES
SEQ ID NO: 43 - AMINO ACID SEQUENCE OF FLEXIBLE LINKER PEPTIDE
GGGGS
SEQ ID NO: 44 - AMINO ACID SEQUENCE OF EXEMPLARY LINKER
PEPTIDE FOR USE BETWEEN FUNCTIONAL DOMAINS
LGGGGSGGGGSGGGGSAAA
SEQ ID NO: 45 - AMINO ACID SEQUENCE OF EXEMPLARY LINKER
PEPTIDE FOR USE BETWEEN FUNCTIONAL DOMAINS
LSGGGGSGGGGSGGGGSGGGGS
SEQ ID NO: 46 - AMINO ACID SEQUENCE OF EXEMPLARY LINKER
PEPTIDE FOR USE BETWEEN FUNCTIONAL DOMAINS (helical)
LAEAAAKEAAAKEAAAKE
SEQ ID NO: 47 - AMINO ACID SEQUENCE OF EXEMPLARY LINKER
PEPTIDE FOR USE BETWEEN FUNCTIONAL DOMAINS
LAEAAAKEAAAKEAAAKAAA
SEQ ID NO: 48 - AMINO ACID SEQUENCE OF EXEMPLARY LINKER
PEPTIDE FOR USE BETWEEN FUNCTIONAL DOMAINS
LAEAAAKEAAAKEAAAKEAAAKAAA
SEQ ID NO: 49 - AMINO ACID SEQUENCE OF EXEMPLARY LINKER
PEPTIDE FOR USE BETWEEN FUNCTIONAL DOMAINS
LAEAAAKEAAAKAAANGSLAEAAAKEAAAKAAA
SEQ ID NO: 50 - AMINO ACID SEQUENCE OF EXEMPLARY LINKER
PEPTIDE FOR USE BETWEEN FUNCTIONAL DOMAINS
AEAAAKEAAAKEAAAKEAAAKALEAEAAAKEAAAKEAAAKEAAAKA
SEQ ID NO: 51 - AMINO ACID SEQUENCE OF EXEMPLARY LINKER
PEPTIDE FOR USE BETWEEN FUNCTIONAL DOMAINS
DKTHTCPPCPAPELLGGPSVFLFPPKPKDT
SEQ ID NO: 52 - AMINO ACID SEQUENCE OF EXEMPLARY LINKER
PEPTIDE FOR USE BETWEEN FUNCTIONAL DOMAINS
DKTHTSPPSPAPELLGGPSV LFPPKPKDT
SEQ ID NO: 53 - AMINO ACID SEQUENCE OF EXEMPLARY LINKER
PEPTIDE FOR USE BETWEEN FUNCTIONAL DOMAINS
DKTHTCPPCPAPELLGGP
SEQ ID NO: 54 - AMINO ACID SEQUENCE OF EXEMPLARY LINKER
PEPTIDE FOR USE BETWEEN FUNCTIONAL DOMAINS
DKTHTSPPSPAPELLGGP
SEQ ID NO: 55 - AMINO ACID SEQUENCE OF EXEMPLARY LINKER
PEPTIDE FOR USE BETWEEN FUNCTIONAL DOMAINS
DKTHTCPPCPAPEL
SEQ ID NO: 56 - AMINO ACID SEQUENCE OF EXEMPLARY LINKER
PEPTIDE FOR USE BETWEEN FUNCTIONAL DOMAINS
DKTHTSPPSPAPEL
SEQ ID NO: 57 - AMINO ACID SEQUENCE OF EXEMPLARY LINKER
PEPTIDE FOR USE BETWEEN FUNCTIONAL DOMAINS
DKTHTSPPSPAPELLGGPNGSDKTHTSPPSPAPELLGGP
SEQ ID NO: 58 - AMINO ACID SEQUENCE OF EXEMPLARY LINKER
PEPTIDE FOR USE BETWEEN FUNCTIONAL DOMAINS
EPKSCDKTHTCPPCPAPELLGGP
SEQ ID NO: 59 - AMINO ACID SEQUENCE OF EXEMPLARY LINKER PEPTIDE FOR
USE BETWEEN FUNCTIONAL DOMAINS
EPKSSDKTHTSPPSPAPELLGGP
SEQ ID NO: 60 - AMINO ACID SEQUENCE OF EXEMPLARY LINKER
PEPTIDE FOR USE BETWEEN FUNCTIONAL DOMAINS
ESKYGPPCPSCPAPEFLGGP
SEQ ID NO: 61 - AMINO ACID SEQUENCE OF EXEMPLARY LINKER
PEPTIDE FOR USE BETWEEN FUNCTIONAL DOMAINS
ESKYGPPSPSSPAPEFLGGP
SEQ ID NO: 62 - AMINO ACID SEQUENCE OF EXEMPLARY LINKER
PEPTIDE FOR USE BETWEEN FUNCTIONAL DOMAINS
PAPAPAP PAPAPAPPAPAPAPPAPAPAP
SEQ ID NO: 63 - AMINO ACID SEQUENCE OF EXEMPLARY LINKER
PEPTIDE FOR USE BETWEEN FUNCTIONAL DOMAINS
APKPAPEPKPAPKPAPEPKPAPKPAPEPKPAPKPAPEPKP
SEQ ID NO: 64 - AMINO ACID SEQUENCE OF EXEMPLARY LINKER
PEPTIDE FOR USE BETWEEN FUNCTIONAL DOMAINS
GSAGSAAGSGEFGSAGSAAGSGEFGSAGSAAGSGEF
SEQ ID NO: 65 - AMINO ACID SEQUENCE OF EXEMPLARY LINKER
PEPTIDE FOR USE BETWEEN FUNCTIONAL DOMAINS
GGGGSLVPRGSGGGG
SEQ ID NO: 66 - AMINO ACID SEQUENCE OF EXEMPLARY LINKER
PEPTIDE FOR USE BETWEEN FUNCTIONAL DOMAINS
GGGSGGGSNTSGGGS
SEQ ID NO: 67 - AMINO ACID SEQUENCE OF EXEMPLARY LINKER
PEPTIDE FOR USE BETWEEN FUNCTIONAL DOMAINS
GGGSNTTGGGS
SEQ ID NO: 68 - AMINO ACID SEQUENCE OF EXEMPLARY LINKER
PEPTIDE FOR USE BETWEEN FUNCTIONAL DOMAINS
GGGSNASGGGS
SEQ ID NO: 69 - AMINO ACID SEQUENCE OF EXEMPLARY LINKER
PEPTIDE FOR USE BETWEEN FUNCTIONAL DOMAINS
GGGGSGGGGSNTTGGGGSGGGGS
SEQ ID NO: 70 - AMINO ACID SEQUENCE OF EXEMPLARY LINKER
PEPTIDE FOR USE BETWEEN FUNCTIONAL DOMAINS
GGGS
SEQ ID NO: 71 - AMINO ACID SEQUENCE OF EXEMPLARY LINKER
PEPTIDE FOR USE BETWEEN FUNCTIONAL DOMAINS
GGGGSGGGGSNTTGGGGSGGGGS
FUSION PROTEIN SEQUENCES (INCLUDING LIGAND
BINDING/EXTRACELLULAR DOMAINS)
SEQ ID NO: 72 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 73 - AMINO ACID SEQUENCE OF A FUSION PROTEIN
COMPRISING IL-17 RA-Fc/IgG1-LINKER-BAFFR-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 74 - AMINO ACID SEQUENCE OF A FUSION PROTEIN
COMPRISING IL-17 RA-Fc/IgG1-LINKER-BAFFR-LINKER-BAFFR-LINKER-
BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 75 - AMINO ACID SEQUENCE OF A FUSION PROTEIN
COMPRISING IL-17 AR-Fc/IgG1-LINKER-IFN gamma RI
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLWGSTGDEPKSC
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
EMGTADLGPSSVPTPTNVTIESYNMNPIVYWEYQIMPQVPVFTVEVKNYGVKNSEWIDC
INISHHYCNISDHVGDPSNSLWVRVKARVGQKESAYAKSEEFAVCRDGKIGPPKLDIRK
E
EKQIMIDIFHPSVFVNGDEQEVDYDPETTCYIRVYNVYVRMNGSEIQYKILTQKEDDCDE
IQCQLAIPVSSLNSQYCVSAEGVLHVWGVTTEKSKEVCITIFNSSIKG
SEQ ID NO: 76 - AMINO ACID SEQUENCE OF A FUSION PROTEIN
COMPRISING TNFR2-Fc/IgG1-LINKER-BAFFR-LINKER-BAFFR
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
GSTGDEPKSC DKTHTCPPCP APELLGGPSV FLFPPKPKDT LMISRTPEVT
CVVVDVSHED PEVKFNWYVD GVEVHNAKTK PREEQYNSTY RVVSVLTVLH
QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL
TVDKSRWQQG NVFSCSVMHE ALHNHYTQKS LSLSPGK
LAEAAAKEAAAKEAAAKE
RGPRSLRGRDAPAPTPCV PAECFDLLVR HCVACGLLRTPRPKPAGASS PAPRTALQPQ
ES LAEAAAKEAAAKEAAAKE
RGPRSLRG RDAPAPTPCVPAECFDLLVR HCVACGLLRTPRPKPAGASS PAPRTALQPQ
ES
SEQ ID NO: 77 - AMINO ACID SEQUENCE OF A FUSION PROTEIN
COMPRISING TNFR5-Fc/IgG1-LINKER-BAFFR-LINKER-BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLRGSTGD
EPKSC DKTHTCPPCP APELLGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSREEMTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
GGGGSGGGGS
RGPRSLRGRDAPAPTPCV PAECFDLLVR HCVACGLLRTPRPKPAGASS
PAPRTALQPQES
GGGGSGGGGS
RGPRSLRG RDAPAPTPCVPAECFDLLVR HCVACGLLRTPRPKPAGASS
PAPRTALQPQES
SEQ ID NO: 78 - AMINO ACID SEQUENCE OF A FUSION PROTEIN
COMPRISING IFN gammaR-Fc/IgG1-LINKER-BCMA
EMGTADLGPSSVPTPTNVTIESYNMNPIVYWEYQIMPQVPVFTVEVKNYGVKNSEWID
ACINISHHYCNISDHVGDPSNSLWVRVKARVGQKESAYAKSEEFAVCRDGKIGPPKLDI
RKEEKQIMIDIFHPSVFVNGDEQEVDYDPETTCYIRVYNVYVRMNGSEIQYKILTQKEDD
CDEIQCQLAIPVSSLNSQYCVSAEGVLHVWGVTTEKSKEVCITIFNSSIKG
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
LQMAGQCSQNEYFDSLLHACIPCQLRCSSNTPPLTCQRYCNASVTNSVKGTNA
SEQ ID NO: 79 - AMINO ACID SEQUENCE OF A FUSION PROTEIN
COMPRISING IL-17 RA-Fc/IgG1-LINKER-TACI
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 80 - AMINO ACID SEQUENCE OF A FUSION PROTEIN
COMPRISING IL-17 RA-Fc/IgG1-LINKER-BCMA
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
LQMAGQCSQNEYFDSLLHACIPCQLRCSSNTPPLTCQRYCNASVTNSVKGTNA
SEQ ID NO: 81 - AMINO ACID SEQUENCE OF A FUSION PROTEIN
COMPRISING CTLA-4-Fc/IgG1-LINKER-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 82 - AMINO ACID SEQUENCE OF A FUSION PROTEIN
COMPRISING CTLA-4-Fc/IgG1-LINKER-BAFFR-LINKER-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 83 - AMINO ACID SEQUENCE OF A FUSION PROTEIN
COMPRISING CTLA-4-Fc/IgG1-LINKER-BAFFR-LINKER-BAFFR-LINKER-
BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 84 - AMINO ACID SEQUENCE OF A FUSION PROTEIN
COMPRISING TNFR2-Fc/IgG1-LINKER-BAFFR
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 85 - AMINO ACID SEQUENCE OF A FUSION PROTEIN
COMPRISING Ox40R-Fc/IgG1-LINKER-BAFFR
LHCVGDTYPSNDRCCHECRPGNGMVSRCSRSQNTVCRPCGPGFYNDVVSSKPCKPCTW
CNLRSGSERKQLCTATQDTVCRCRAGTQPLDSYKPGVDCAPCPPGHFSPGDNQACKPW
TNCTLAGKHTLQPASNSSDAICEDRDPPATQPQETQGPPARPITVQPTEAWPRTSQGPST
RPVEVPGGRA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 86 - AMINO ACID SEQUENCE OF A FUSION PROTEIN
COMPRISING IFNGR1-Fc/IgG1-LINKER-BAFFR
EMGTADLGPSSVPTPTNVTIESYNMNPIVYWEYQIMPQVPVFTVEVKNYGVKNSEWID
ACINISHHYCNISDHVGDPSNSLWVRVKARVGQKESAYAKSEEFAVCRDGKIGPPKLDI
RKEEKQIMIDIFHPSVFVNGDEQEVDYDPETTCYIRVYNVYVRMNGSEIQYKILTQKEDD
CDEIQCQLAIPVSSLNSQYCVSAEGVLHVWGVTTEKSKEVCITIFNSSIKG
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 87 - AMINO ACID SEQUENCE OF A FUSION PROTEIN
COMPRISING ICOSR-Fc/IgG1-LINKER-BAFFR
EINGSANYEMFIFHNGGVQILCKYPDIVQQFKMQLLKGGQILCDLTKTKGSGNTVSIKSL
KFCHSQLSNNSVSFFLYNLDHSHANYYFCNLSIFDPPPFKVTLTGGYLHIYESQLCCQLK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 88 - AMINO ACID SEQUENCE OF A FUSION PROTEIN
COMPRISING IL21 RECEPTOR-LINKER-COMMON GAMMA CHAIN-
LINKER-Fc/IgG1-LINKER-BAFFR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
LAEAAAKEAAAKE
SYDSPDYTDESCTFKISLRNFRSILSWELKNHSIVPTHYTLLYTIMSKPEDLKVVKNCANT
TRSFCDLTDEWRSTHEAYVTVLEGFSGNTTLFSCSHNFWLAIDMSFEPPEFEIVGFTNHIN
VMVKFPSIVEEELQFDLSLVIEEQSEGIVKKHKPEIKGNMSGNFTYIIDKLIPNTNYCVSV
YLEHSDEQAVIKSPLKCTLLPPGQESESAESAKDKTHTCPPCPAPELLGGPSVFLFPPKPK
DTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFS
CSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 89 - AMINO ACID SEQUENCE OF A FUSION PROTEIN
COMPRISING IL21R-GAMMA CHAIN-LINKER-Fc/IgG1-LINKER-BAFFR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
LAEAAAKEAAAKEAAAKE
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
ATQMLKLQNLVIPWAPENLTLHKLSERQLELNWNNRFLNHCLEHLVQYRTDWDHSWT
EQSVDYRHKFSLPSVDGQKRYTFRVRSRFNPLCGSAQHWSEWSHPIHWGSNTSKENPFL
FALEA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 90 - AMINO ACID SEQUENCE OF A FUSION PROTEIN
COMPRISING IL-17 RA-Fc/IgG1-LINKER-BAFFR-LINKER-TACI
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 91 - AMINO ACID SEQUENCE OF A FUSION PROTEIN
COMPRISING CTLA-4-Fc/IgG1-LINKER-BAFFR-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPGASSPAPRTALQPQESV
GAGAGEAALPLPGLL
LAEAAAKEAAAKEAAAKE
LQMAGQCSQNEYFDSLLHACIPCQLRCSSNTPPLTCQRYCNASVTNSVKGTNA
SEQ ID NO: 92 - AMINO ACID SEQUENCE OF A FUSION PROTEIN
COMPRISING CTLA-4-Fc/IgG1-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKE
LQMAGQCSQNEYFDSLLHACIPCQLRCSSNTPPLTCQRYCNASVTNSVKGTNA
SEQ ID NO: 93 - AMINO ACID SEQUENCE OF A FUSION PROTEIN
COMPRISING CTLA-4-Fc/IgG1-linker-BCMA-linker-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKE
LQMAGQCSQNEYFDSLLHACIPCQLRCSSNTPPLTCQRYCNASVTNSVKGTNA
GGGGSGGGGS
LQMAGQCSQNEYFDSLLHACIPCQLRCSSNTPPLTCQRYCNASVTNSVKGTNA
SEQ ID NO: 94 - AMINO ACID SEQUENCE OF A FUSION PROTEIN
COMPRISING CTLA-4-Fc/IgG1-LINKER-BCMA-LINKER-BCMA-LINKER-
BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
LQMAGQCSQNEYFDSLLHACIPCQLRCSSNTPPLTCQRYCNASVTNSVKGTNA
GGGGSGGGGS
LQMAGQCSQNEYFDSLLHACIPCQLRCSSNTPPLTCQRYCNASVTNSVKGTNA
GGGGSGGGGS
LQMAGQCSQNEYFDSLLHACIPCQLRCSSNTPPLTCQRYCNASVTNSVKGTNA
SEQ ID NO: 95 - AMINO ACID SEQUENCE OF A FUSION PROTEIN
COMPRISING CTLA-4-Fc/IgG4-LINKER-BAFFR-LINKER-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
NVDHKPS NTKVDKRVES KYGPPCPPCP APEFLGGPSV
FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT
LPPSQEEMTK
NQVSLTCLVK GFYPSDIAVE WESNGQPENN YKTTPPVLDS DGSFFLYSRL
TVDKSRWQEG
NVFSCSVMHE ALHNHYTQKS LSLSLGKGGGGSGGGGS
RGPRSLRGRDAPAPTPCVPAECFDLLVRHCVACGLLRTPRPKPAGASSPAPRTALQPQES
VGAGAGEAALPLPGLLGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 96 - AMINO ACID SEQUENCE OF A FUSION PROTEIN
COMPRISING TNFR2-Fc/IgG1-LINKER-BAFFR-LINKER-BCMA
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKE
RGPRSLRGRDAPAPTPCVPAECFDLLVRHCVACGLLRTPRPKPGASSPAPRTALQPQESV
GAGAGEAALPLPGLL GGGGGGGGS
LQMAGQCSQNEYFDSLLHACIPCQLRCSSNTPPLTCQRYCNASVTNSVKGTNA
SEQ ID NO: 97 - AMINO ACID SEQUENCE OF A FUSION PROTEIN
COMPRISING Ox40R-Fc/IgG1-LINKER-BCMA
LHCVGDTYPSNDRCCHECRPGNGMVSRCSRSQNTVCRPCGPGFYNDVVSSKPCKPCTW
CNLRSGSERKQLCTATQDTVCRCRAGTQPLDSYKPGVDCAPCPPGHFSPGDNQACKPW
TNCTLAGKHTLQPASNSSDAICEDRDPPATQPQETQGPPARPITVQPTEAWPRTSQGPST
RPVEVPGGRA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKE
LQMAGQCSQNEYFDSLLHACIPCQLRCSSNTPPLTCQRYCNASVTNSVKGTNA
SEQ ID NO: 98 - AMINO ACID SEQUENCE OF A FUSION PROTEIN
COMPRISING IFNGR1-Fc/IgG1-LINKER-BCMA
EMGTADLGPSSVPTPTNVTIESYNMNPIVYWEYQIMPQVPVTVEVKNYGVKNSEWIDCI
NISHHYCNISDHVGDPSNSLWVRVKARVGQKESAYAKSEEFAVCRDGKIGPPKLDIRKE
EKQIMIDIFHPSVFVNGDEQEVDYDPETTCYIRVYNVYVRMNGSEIQYKILTQKEDDCDE
IQCQLAIPVSSLNSQYCVSAEGVLHVWGVTTEKSKEVCITIFNSSIKG
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
LQMAGQCSQNEYFDSLLHACIPCQLRCSSNTPPLTCQRYCNASVTNSVKGTNA
SEQ ID NO: 99 - AMINO ACID SEQUENCE OF A FUSION PROTEIN
COMPRISING ICOSR-Fc/IgG1-LINKER-BCMA-LINKER-BAFFR
EINGSANYEMFIFHNGGVQILCKYPDIVQQFKMQLLKGGQILCDLTKTKGSGNTVSIKSL
KFCHSQLSNNSVSFFLYNLDHSHANYYFCNLSIFDPPPFKVTLTGGYLHIYESQLCCQLK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
LQMAGQCSQNEYFDSLLHACIPCQLRCSSNTPPLTCQRYCNASVINSVKGTNA
LAEAAAKEAAAKEAAAKAAA
RGPRSLRGRDAPAPTPCVPAECFDLLVRHCVACGLLRTPRPKPAGASSPAPRTALQPQES
VGAGAGEAALPLPGLL
SEQ ID NO: 100 - AMINO ACID SEQUENCE OF A FUSION PROTEIN
COMPRISING IFNAR2 (high affinity)-Fc/IgG1-LINKER-BAFFR
ISYDSPDYTDESCTFKISLRNFRSILSWELKNHSIVPTHYTLLYTIMSKPEDLKVVKNCA
NTTRSFCDLTDEWRSTHEAYVTVLEGFSGNTTLFSCSHNFWLAIDMSFEPPEFEIVGFTN
HINVMVKFPSIVEEELQFDLSLVIEEQSEGIVKKHKPEIKGNMSGNFTYIIDKLIPNTNY
CVSVYLEHSDEQAVIKSPLKCTLLPPGQESESAESAK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKAAA
RGPRSLRGRDAPAPTPCVPAECFDLLVRHCVACGLLRTPRPKPAGASSPAPRTALQPQES
VGAGAGEAALPLPGLL
SEQ ID NO: 101 - AMINO ACID SEQUENCE OF A FUSION PROTEIN
COMPRISING IL21R-linker-gammachain-Fc/IgG1-LINKER-BAFFR-LINKER-
BAFFR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
GGGGSGGGGSGGGGS
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
ATQMLKLQNLVIPWAPENLTLHKLSERQLELNWNNRFLNHCLEHLVQYRTDWDHSWT
EQSVDYRHKFSLPSVDGQKRYTFRVRSRFNPLCGSAQHWSEWSHPIHWGSNTSKENPFL
FALEA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 102 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
EPKSSDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 103 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSRDELTK NQVSLTCLVK GFYPSDIAVE WESNGQPENN
YKTTPPVLDS
DGSFFLYSKL TVDKSRWQQG NVFSCSVLHE ALHSHYTQKS LSLSPGK
LAEAAAKEAAAKEAAAKAAA
RGPRSLRGRDAPAPTPCVPAECFDLLVRHCVACGLLRTPRPKPAGASSPAPRTALQPQES
VGAGAGEAALPLPGLL
SEQ ID NO: 104 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCP APELLGGPDV FLFPPKPKDT LMISRTPEVT 1DVSHED PEVKFNWYVD
GVEVHNAKTK PREEQYNSTY RVVSVLTVLH QDWLNGKEYK CKVSNKALPA
PEEKTISKAK
GQPREPQVYT LPPSRDELTK NQVSLTCLVK GFYPSDIAVE WESNGQPENN
YKTTPPVLDS
GGGGSGGGGSNTTGGGGSGGGGS
DGSFFLYSKL TVDKSRWQQG NVFSCSVMHE ALHNHYTQKS LSLSPGK
RGPRSLRGRDAPAPTPCVPAECFDLLVRHCVACGLLRTPRPKPAGASSPAPRTALQPQES
VGAGAGEAALPLPGLL
SEQ ID NO: 105 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPEAAGGPSVFLFPPKPKDTLMISRTPEVTCVVVDVSHEDPEVKFNWYV
D
GVEVHNAKTKPREEQYNSTYRVVSVLTVLHQDWLNGKEYKCKVSNKALPAPIEKTISK
AK
GQPREPQVYTLPPSRDELTKNQVSLTCLVKGFYPSDIAVEWESNGQPENNYKTTPPVLD
S
DGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 106 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
D
GVEVHAAKTKPREEQYNSTYRVVSVLTVLHQDWLNGKEYKCKVSNKALPAPIEKTISK
AK
GQPREPQVYTLPPSRDELTKNQVSLTCLVKGFYPSDIAVE
WESNGQPENNYKTTPPVLDS
DGSFFLYSKLTVDKSRWQQGNVFSCSVMHEALHNHYTQKSLSLSPGK
GGGGSGGGGSNTTGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 107 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
D
GVEVHAAKTKPREEQYNSTYRVVSVLTVLH
QDWLNGKEYKCKVSNKALPAPIEKTISKAK
GQPREPQVYTLPPSRDELTKNQVSLTCLVKGFYPSDIAVEWESNGQPENNYKTTPPVLS
DGSFFLYSKLTVDKSRWQQGNVFSCSVMHEALHNHYTQKSLSLSPGK
LAEAAAKEAAAKEAAAKAAA
RGPRSLRGRDAPAPTPCVPAECFDLLVRHCVACGLLRTPRPKPAGASSPAPRTALQPQES
VGAGAGEAALPLPGLL
SEQ ID NO: 108 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPDVFLFPPKPKDTLMISRTPEVT
CVVVDVSHEDPEVKFNWYVD
GVEVHNAKTK PREEQYNSTYRVVSVLTVLH QDWLNGKEYK
CKVSNKALPAPEEKTISKAK
GQPREPQVYTLPPSRDELTKNQVSLTCLVK GFYPSDIAVE
WESNGQPENNYKTTPPVLDS
DGSFFLYSKLTVDKSRWQQG NVFSCSVMHE ALHNHYTQKS LSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 109 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
EPKSSDKTHTCPSCSAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
LAEAAAKEAAAKEAAAKAAA
RGPRSLRGRDAPAPTPCVPAECFDLLVRHCVACGLLRTPRPKPAGASSPAPRTALQPQES
VGAGAGEAALPLPGLL
SEQ ID NO: 110 - AMINO ACID SEQUENCE OF A FUSION PROTEIN
COMPRISING IL-17 RA-Fc/IgG4-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
KYGPPCPPCPAPEFLGGPSVFLFPPKPKDTLMISRTPEVTCVVVDVSQED
PEVQFNWYVD GVEVHNAKTK PREEQFNSTYRVVSVLTVLH
QDWLNGKEYKCKVSNKGLPSSIEKTISKAK
GQPREPQVYTLPPSQEEMTKNQVSLTCLVK
GFYPSDIAVEWESNGQPENNYKTTPPVLDS
DGSFFLYSRLTVDKSRWQEGNVFSCSVMHEALHNHYTQKSLSLSLGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 111 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 112 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 113 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSNSTGGGGSGGGS
RGPRSLRGRDAPAPTPCVPAECFDLLVRHCVACGLLRTPRPKPAGASSPAPRTALQPQES
VGAGAGEAALPLPGLL
SEQ ID NO: 114 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
EPKSSDKTHTSPSSSAPEAAGGPSVFL
RGPRSLRGRDAPAPTPCVPAECFDLLVRHCVACGLLRTPRPKPAGASSPAPRTALQPQES
VGAGAGEAALPLPGLL
SEQ ID NO: 115 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
DKTHTSPPSPAPELLGGPSVFLFPPKPKD
RGPRSLRGRDAPAPTPCVPAECFDLLVRHCVACGLLRTPRPKPAGASSPAPRTALQPQES
VGAGAGEAALPLPGLL
SEQ ID NO: 116 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
EAEAAAKEAAAKEAAAKEAAAKA
RGPRSLRGRDAPAPTPCVPAECFDLLVRHCVACGLLRTPRPKPAGASSPAPRTALQPQES
VGAGAGEAALPLPGLL
SEQ ID NO: 117 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSLVPRGSGGGG
RGPRSLRGRDAPAPTPCVPAECFDLLVRHCVACGLLRTPRPKPAGASSPAPRTALQPQES
VGAGAGEAALPLPGLL
SEQ ID NO: 118 - AMINO ACID SEQUENCE OF A FUSION PROTEIN
COMPRISING IL-17 RA-Fc/IgG1-LINKER-BAFFR-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
GSTGDEPKSC DKTHTCPPCP APELLGGPSV FLFPPKPKDT LMISRTPEVT
CVVVDVSHED PEVKFNWYVD GVEVHNAKTK PREEQYNSTY RVVSVLTVLH
QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL
TVDKSRWQQG NVFSCSVMHE ALHNHYTQKS LSLSPGK
GGGGSGGGGSRGPRSLRGRDAPAPTPCVPAECFDLLVRHCVACGLLRTPRPKPAGASSP
APRTALQPQESVGAGAGEAALPLPGLLGGGGSLVPRGSGGGGRGPRSLRGRDAPAPTPC
VPAECFDLLVRHCVACGLLRTPRPKPAGASSPAPRTALQPQESVGAGAGEAALPLPGLL
SEQ ID NO: 119 - AMINO ACID SEQUENCE OF A FUSION PROTEIN
COMPRISING IL-17 RA-Fc/IgG1-LINKER-BAFFR-LINKER-BAFFR-LINKER
BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSLVPRGSGGGG
RGPRSLRGRDAPAPTPCVPAECFDLLVRHCVACGLLRTPRPKPAGASSPAPRTALQPQES
VGAGAGEAALPLPGLL
GGSGGHMGSGG
RGPRSLRGRDAPAPTPCVPAECFDLLVRHCVACGLLRTPRPKPAGASSPAPRTALQPQES
VGAGAGEAALPLPGLL
SEQ ID NO: 120 - AMINO ACID SEQUENCE OF A FUSION PROTEIN
COMPRISING BAFFR-Fc/IgG1-LINKER-BAFFR-LINKER-TACI
SLRGRDAPAPTPCVPAECFDLLVRHCVACGLLRTPRPKPAGASSPAPRTALQPQESVGA
GAGEAALPLPGLL
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSLVPRGSGGGG
RGPRSLRGRDAPAPTPCVPAECFDLLVRHCVACGLLRTPRPKPAGASSPAPRTALQPQES
VGAGAGEAALPLPGLL
GGSGGHMGSGG
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQV
SEQ ID NO: 121 - AMINO ACID SEQUENCE OF A FUSION PROTEIN
COMPRISING IL-21R-Fc(1)-LINKER-BAFFR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLTSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 122 - AMINO ACID SEQUENCE OF A FUSION PROTEIN
COMPRISING IL22R alpha -linker-IL10R beta-Fc-linker-BAFFR-linker-BAFFR
HAPEDPSDLLQHVKFQSSNFENILTWDSGPEGTPDTVYSIEYKTYGERDWVAKKGCQRI
TRKSCNLTVETGNLTELYYARVTAVSAGGRSATKMTDRFSSLQHTTLKPPDVTCISKVR
SIQMIVHPTPTPIRAGDGHRLTLEDIFHDLFYHLELQVNRTYQMHLGGKQREYEFFGLTP
DTEFLGTIMICVPTWAKESAPYMCRVKTLPDRTWTGGGGSGGGGSGGGGS
MVPPPENVRMNSVNFKNILQWESPAFAKGNLTFTAQYLSYRIFQDKCMNTTLTECDFSS
LSKYGDHTLRVRAEFADEHSDWVNITFCPVDDTIIGPPGMQVEVLADSLHMRFLAPKIE
NEYETWTMKNVYNSWTYNVQYWKNGTDEKFQITPQYDFEVLRNLEPWTTYCVQVRG
FLPDRNKAGEWSEPVCEQTTHDETVPS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLYCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 123 - AMINO ACID SEQUENCE OF A FUSION PROTEIN
COMPRISING PD-1-Fc/IgG4-LINKER-CTLA-4
FLDSPDRPWNPPTFSPALLVVTEGDNATFTCSFSNTSESFVLNWYRMSPSNQTDKLAAFP
EDRSQPGQDCRFRVTQLPNGRDFHMSVVRARRNDSGTYLCGAISLAPKAQIKESLRAEL
RVTERRAEVPTAHPSPSPRPAGQFQTLVDHKPS NTKVDKRVES KYGPPCPPCP
APEFLGGPSV
FLFPPKPKDT LMISRTPEVT CVVVDVSQED PEVQFNWYVD GVEVHNAKTK
PREEQFNSTYRVVSVLTVLH QDWLNGKEYK CKVSNKGLPS SIEKTISKAK
GQPREPQVYT LPPSQEEMTKNQVSLTCLVK GFYPSDIAVE
WESNGQPEGGGGSGGGGS
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
SEQ ID NO: 124 - AMINO ACID SEQUENCE OF A FUSION PROTEIN
COMPRISING LAG3-Fc/IgG4-LINKER-CTLA-4
LQPGAEVPVVWAQEGAPAQLPCSPTIPLQDLSLLRRAGVTWQHQPDSGPPAAAPGHPL
APGPHPAAPSSWGPRPRRYTVLSVGPGGLRSGRLPLQPRVQLDERGRQRGDFSLWLRPA
RRADAGEYRAAVHLRDRALSCRLRLRLGQASMTASPPGSLRASDWVILNCSFSRPDRPA
SVHWFRNRGQGRVPVRESPHHHLAESFLFLPQVSPMDSGPWGCILTYRDGFNVSIMYNL
TVLGLEPPTPLTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLGKLLCEVTPVSG
QERFVWSSLDTPSQRSFSGPWLEAQEAQLLSQPWQCQLYQGERLLGAAVYFTELSSPGA
QRSGRAPGALPAGHL
NVDHKPSNTK VDKRVESKYG PPCPPCPAPE FLGGPSVFLFPPKPKDTLMI
SRTPEVTCVV
VDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYT
QKSLSLSLGK
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
SEQ ID NO: 125 - AMINO ACID SEQUENCE OF A FUSION PROTEIN
COMPRISING TIM-3 EXTRACELLULAR DOMAIN-Fc-IgG4--LINKER-PD1
SEVEYRAEVGQNAYLPCFYTPAAPGNLVPVCWGKGACPVFECGNVVLRTDERDVNYW
TSRYWLNGDFRKGDVSLTIENVTLADSGIYCCRIQIPGIMNDEKFNLKLVIKPAKVTPAP
TRQRDFTAAFPRMLTTRGHGPAETQTLGSLPDINLTQISTLANELRDSRLANDLRDSGAT
IRIG
DHKPSNTKVDKRVESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHYTQK
GGGGSGGGGS
FLDSPDRPWNPPTFSPALLVVTEGDNATFTCSFSNTSESFVLNWYRMSPSNQTDKLAAFP
EDRSQPGQDCRFRVTQLPNGRDFHMSVVRARRNDSGTYLCGAISLAPKAQIKESLRAEL
RVTERRAEVPTAHPSPSPRPAGQFQTLV
SEQ ID NO: 126 - AMINO ACID SEQUENCE OF A FUSION PROTEIN
COMPRISING LAG-3-Fc/IgG4-LINKER-PD1
LQPGAEVPVVWAQEGAPAQLPCSPTIPLQDLSLLRRAGVTWQHQPDSGPPAAAPGHPL
AP
GPHPAAPSSWGPRPRRYTVLSVGPGGLRSGRLPLQPRVQLDERGRQRGDFSLWLRPARR
A
DAGEYRAAVHLRDRALSCRLRLRLGQASMTASPPGSLRASDWVILNCSFSRPDRPASVH
W
FRNRGQGRVPVRESPHHHLAESFLFLPQVSPMDSGPWGCILTYRDGFNVSIMYNLTVLG
L
EPPTPLTVYAGAGSRVGLPCRLPAGVGTRSFLTAKWTPPGGGPDLLVTGDNGDFTLRLE
D
VSQAQAGTYTCHIHLQEQQLNATVTLAIITVTPKSFGSPGSLGKLLCEVTPVSGQERFVW
SSLDTPSQRSFSGPWLEAQEAQLLSQPWQCQLYQGERLLGAAVYFTELSSPGAQRSGRA
P
GALPAGHL
NVDHKPS NTKVDKRVES KYGPPCPPCP APEFLGGPSVFLFPPKPKDT LMISRTPEVT
CVVVDVSQED PEVQFNWYVD GVEVHNAKTK PREEQFNSTYRVVSVLTVLH
QDWLNGKEYK CKVSNKGLPS SIEKTISKAK GQPREPQVYT
LPPSQEEMTKNQVSLTCLVK GFYPSDIAVE WESNGQPENN YKTTPPVLDS
DGSFFLYSRL TVDKSRWQEGNVFSCSVMHE ALHNHYTQKS LSLSLGKGGGGSGGGGS
FLDSPDRPWNPPTFSPALLVVTEGDNATFTCSFSNTSESFVLNWYRMSPSNQTDKLAAFP
EDRSQPGQDCRFRVTQLPNGRDFHMSVVRARRNDSGTYLCGAISLAPKAQIKESLRAEL
R VTERRAEVPTAHPSPSPRPAGQFQTLV
SEQ ID NO: 127 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 128 - AMINO ACID SEQUENCE OF A FUSION PROTEIN
COMPRISING IL-17 RA-Fc/IgG1-BAFFR-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIP APRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
RGPRSLRGRDAPAPTPCVPAECFDLLVRHCVACGLLRTPRPKPAGASSPAPRTALQPQES
VGAGAGEAALPLPGLL
GGGGS
RGPRSLRGRDAPAPTPCVPAECFDLLVRHCVACGLLRTPRPKPAGASSPAPRTALQPQES
VGAGAGEAALPLPGLL
SEQ ID NO: 129 - AMINO ACID SEQUENCE OF A FUSION PROTEIN
COMPRISING IL-17 RA-Fc/IgG1-LINKER-BAFFR-LINKER-BAFFR-LINKER-
BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
GGGGS
RGPRSLRGRDAPAPTPCVPAECFDLLVRHCVACGLLRTPRPKPAGASSPAPRTALQPQES
VGAGAGEAALPLPGLL
GGGGS
RGPRSLRGRDAPAPTPCVPAECFDLLVRHCVACGLLRTPRPKPAGASSPAPRTALQPQES
VGAGAGEAALPLPGLL
SEQ ID NO: 130 - AMINO ACID SEQUENCE OF A FUSION PROTEIN
COMPRISING IL-17 AR-Fc-LINKER-IFN gamma RI
LRLLDHRALVCSQPGLNCTVKNSTCLDD
SWIHPRNLTPSSPKDLQIQLHFAHTQQGDLFPVAHIEWTLQTDASILYLEGAELSVLQLN
TNERLCVRFEFLSKLRHHHRRWRFTFSHFVVDPDQEYEVTVHHLPKPIPDGDPNHQSKN
FLVPDCEHARMKVTTPCMSSGSLWDPNITVETLEAHQLRVSFTLWNESTHYQILLTSFP
HMENHSCFEHMHHIPAPRPEEFHQRSNVTLTLRNLKGCCRHQVQIQPFFSSCLNDCLRH
SATVSCPEMPDTPEPIPDYMPLWGSTGDEPKSC DKTHTCPPCP APELLGGPSV
FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK
GQPREPQVYT LPPSREEMTK NQVSLTCLVK GFYPSDIAVE WESNGQPENN
YKTTPPVLDS DGSFFLYSKL TVDKSRWQQG NVFSCSVMHE ALHNHYTQKS
LSLSPGK GGGGS
EMGTADLGPSSVPTPTNVTIESYNMNPIVYWEYQIMPQVPVFTVEVKNYGVKNSEWID
AC
INISHHYCNISDHVGDPSNSLWVRVKARVGQKESAYAKSEEFAVCRDGKIGPPKLDIRK
E
EKQIMIDIFHPSVFVNGDEQEVDYDPETTCYIRVYNVYVRMNGSEIQYKILTQKEDDCDE
IQCQLAIPVSSLNSQYCVSAEGVLHVWGVTTEKSKEVCITIFNSSIKG
SEQ ID NO: 131 - AMINO ACID SEQUENCE OF A FUSION PROTEIN
COMPRISING TNFR2-FC-LINKER-BAFFR-LINKER-BAFFR
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
GSTGDEPKSC DKTHTCPPCP APELLGGPSV FLFPPKPKDT LMISRTPEVT
CVVVDVSHED PEVKFNWYVD GVEVHNAKTK PREEQYNSTY RVVSVLTVLH
QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL
TVDKSRWQQG NVFSCSVMHE ALHNHYTQKS LSLSPGK GGGGS
RGPRSLRGRDAPAPTPCV PAECFDLLVR HCVACGLLRTPRPKPAGASS PAPRTALQPQ
ES GGGGSGGGGS
RGPRSLRG RDAPAPTPCVPAECFDLLVR HCVACGLLRTPRPKPAGASS PAPRTALQPQ
ES
SEQ ID NO: 132 - AMINO ACID SEQUENCE OF A FUSION PROTEIN
COMPRISING TNFR5-Fc-LINKER-BAFFR-LINKER-BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCP APELLGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSREEMTK NQVSLTCLVK
GFYPSDIAVE WESNGQPENN YKTTPPVLDS DGSFFLYSKL TVDKSRWQQG
NVFSCSVMHE ALHNHYTQKS LSLSPGK GGGGS
RGPRSLRGRDAPAPTPCV PAECFDLLVR HCVACGLLRTPRPKPAGASS
PAPRTALQPQES GGGGS
RGPRSLRG RDAPAPTPCVPAECFDLLVR HCVACGLLRTPRPKPAGASS
PAPRTALQPQES
SEQ ID NO: 133 - AMINO ACID SEQUENCE OF A FUSION PROTEIN
COMPRISING IFN AR2-Fc-LINKER-BCMA
ISYDSPDYTDESCTFKISLRNFRSILSWELKNHSIVPTHYTLLYTIMSKPEDLKVVKNCAN
TTRSFCDLTDEWRSTHEAYVTVLEGFSGNTTLFSCSHNFWLAIDMSFEPPEFEFVGFTN
HINVMVKFPSIVEEELQFDLSLVIEEQSEGIVKKHKPEIKGNMSGNFTYIIDKLIPNTNY
CVSVYLEHSDEQAVIKSPLKCTLLPPGQESESAESAKDKTHTCPPCPAPELLGGPSVFLFP
PKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQG
NVFSCSVMHEALHNHYTQKSLSLSPGK
GGGGS
LQMAGQCSQNEYFDSLLHACIPCQLRCSSNTPPLTCQRYCNASVTNSVKGTNA
SEQ ID NO: 134 - AMINO ACID SEQUENCE OF A FUSION PROTEIN
COMPRISING IL-17 RA-Fc/IgG1-LINKER-TACI
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 135 - AMINO ACID SEQUENCE OF A FUSION PROTEIN
COMPRISING IL-17 RA-Fc/IgG1-LINKER-BCMA
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVINSVKGTNA
SEQ ID NO: 136 - AMINO ACID SEQUENCE OF A FUSION PROTEIN
COMPRISING CTLA-4-Fc-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 137 - AMINO ACID SEQUENCE OF A FUSION PROTEIN
COMPRISING CTLA-4-Fc-LINKER-BAFFR-LINKER-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPGASSPAPRTALQPQESV
GAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 138 - AMINO ACID SEQUENCE OF A FUSION PROTEIN
COMPRISING CTLA-4-Fc-LINKER-BAFFR-LINKER-BAFFR-LINKER-BAFFR
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 139 - AMINO ACID SEQUENCE OF A FUSION PROTEIN
COMPRISING TNFR2-Fc-LINKER-BAFFR
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 140 - AMINO ACID SEQUENCE OF A FUSION PROTEIN
COMPRISING Ox40R-Fc-LINKER-BAFFR
LHCVGDTYPSNDRCCHECRPGNGMVSRCSRSQNTVCRPCGPGFYNDVVSSKPCKPCTW
CNLRSGSERKQLCTATQDTVCRCRAGTQPLDSYKPGVDCAPCPPGHFSPGDNQACKPW
TNCTLAGKHTLQPASNSSDAICEDRDPPATQPQETQGPPARPITVQPTEAWPRTSQGPST
RPVEVPGGRA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPGASSPAPRTALQPQESV
GAGAGEAALPLPGLL
SEQ ID NO: 141 - AMINO ACID SEQUENCE OF A FUSION PROTEIN
COMPRISING IFNGR1-Fc-LINKER-BAFFR
EMGTADLGPSSVPTPTNVTIESYNMNPIVYWEYQIMPQVPVFTVEVKNYGVKNSEWID
ACINISHHYCNISDHVGDPSNSLWVRVKARVGQKESAYAKSEEFAVCRDGKIGPPKLDI
RKEEKQIMIDIFHPSVFVNGDEQEVDYDPETTCYIRVYNVYVRMNGSEIQYKILTQKEDD
CDEIQCQLAIPVSSLNSQYCVSAEGVLHVWGVTTEKSKEVCITIFNSSIKG
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 142 - AMINO ACID SEQUENCE OF A FUSION PROTEIN
COMPRISING ICOSR-Fc-LINKER-BAFFR
EINGSANYEMFIFHNGGVQILCKYPDIVQQFKMQLLKGGQILCDLTKTKGSGNTVSIKSL
KFCHSQLSNNSVSFFLYNLDHSHANYYFCNLSIFDPPPFKVTLTGGYLHIYESQLCCQLK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 143 - AMINO ACID SEQUENCE OF A FUSION PROTEIN
COMPRISING IFNAR1-LINKER-IFNAR2-Fc-LINKER-BAFFR
KNLKSPQKVEVDIIDDNFILRWNRSDESVGNVTFSFDYQKTGMDNWIKLSGCQNITSTK
CNFSSLKLNVYEEIKLRIRAEKENTSSWYEVDSFTPFRKAQIGPPEVHLEAEDKAIVIHISP
GTKDSVMWALDGLSFTYSLVIWKNSSGVEERIENIYSRHKIYKLSPETTYCLKVKAALL
TSWKIGVYSPVHCIKTTVENELPPPENIEVSVQNQNYVLKWDYTYANMTFQVQWLHAF
LKRNPGNHLYKWKQIPDCENVKTTQCVFPQNVFQKGIYLLRVQASDGNNTSFWSEEIKF
DTEIQAFLLPPVENIRSLSDSFHIYIGAPKQSGNTPVIQDYPLIYEIIFWENTSNAERKIIEKK
TDVTVPNLKPLTVYCVKARAHTMDEKLNKSSVFSDAVCEKTKPGNTSKGGGGSGGGG
SGGGGSGGGGSISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNFWLAIDMSFE
PPEFEIVGFTNHINVMVKFPSIVEEELQFDLSLVIEEQSEGIVKKHKPEIKGNMSGNFTYII
DKLIPNTNYCVSVYLEHSDEQAVIKSPLKCTLLPPGQESESAESAKDKTHTCPPCPAPELL
GGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
GGGGS
RGPRSLRGRDAPAPTPCVPAECFDLLVRHCVACGLLRTPRPKPGASSPAPRTALQPQESV
GAGAGEAALPLPGLL
SEQ ID NO: 144 - AMINO ACID SEQUENCE OF A FUSION PROTEIN
COMPRISING IL21R-LINKER-GAMMA CHAIN-Fc-LINKER-BCMA
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
GGGGSGGGGS
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
ATQMLKLQNLVIPWAPENLTLHKLSERQLELNWNNRFLNHCLEHLVQYRTDWDHSWT
EQSVDYRHKFSLPSVDGQKRYTFRVRSRFNPLCGSAQHWSEWSHPIHWGSNTSKENPFL
FALEA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
SEQ ID NO: 145 - AMINO ACID SEQUENCE OF A FUSION PROTEIN
COMPRISING IL-17 RA-Fc/IgG1-LINKER-BAFFR-LINKER-TACI
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
GGGGSGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 146 - AMINO ACID SEQUENCE OF A FUSION PROTEIN
COMPRISING CTLA-4-Fc-LINKER-BAFFR-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
GGGGSGGGS
LQMAGQCSQNEYFDSLLHACIPCQLRCSSNTPPLTCQRYCNASVTNSVKGTNA
SEQ ID NO: 147 - AMINO ACID SEQUENCE OF A FUSION PROTEIN
COMPRISING CTLA-4-Fc-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
SEQ ID NO: 148 - AMINO ACID SEQUENCE OF A FUSION PROTEIN
COMPRISING CTLA-4-Fc-LINKER-BCMA-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
GGGGSGGGS
LQMAGQCSQNEYFDSLLHACIPCQLRCSSNTPPLTCQRYCNASVTNSVKGTNA
SEQ ID NO: 149 - AMINO ACID SEQUENCE OF A FUSION PROTEIN
COMPRISING CTLA-4-Fc-LINKER-BCMA-LINKER-BCMA-LINKER-BCMA
KAMHVAQPAVVLASSRGIASFVCEYASPGKATEVRVTVLRQADSQVTEVCAATYMMG
NELTFLDDSICTGTSSGNQVNLTIQGLRAMDTGLYICKVELMYPPPYYLGIGNGTQIYVI
DPEPCPDSD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
GGGGSGGGS
LQMAGQCSQNEYFDSLLHACIPCQLRCSSNTPPLTCQRYCNASVTNSVKGTNA
GGGGSGGGS
LQMAGQCSQNEYFDSLLHACIPCQLRCSSNTPPLTCQRYCNASVTNSVKGTNA
SEQ ID NO: 150 - AMINO ACID SEQUENCE OF A FUSION PROTEIN
COMPRISING TNFR2-Fc-LINKER-BAFFR-LINKER-BCMA
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGD
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
GGGGSGGGS
LQMAGQCSQNEYFDSLLHACIPCQLRCSSNTPPLTCQRYCNASVTNSVKGTNA
SEQ ID NO: 151 - AMINO ACID SEQUENCE OF A FUSION PROTEIN
COMPRISING Ox40R-Fc-LINKER-BCMA
LHCVGDTYPSNDRCCHECRPGNGMVSRCSRSQNTVCRPCGPGFYNDVVSSKPCKPCTW
CNLRSGSERKQLCTATQDTVCRCRAGTQPLDSYKPGVDCAPCPPGHFSPGDNQACKPW
TNCTLAGKHTLQPASNSSDAICEDRDPPATQPQETQGPPARPITVQPTEAWPRTSQGPST
RPVEVPGGRA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
SEQ ID NO: 152 - AMINO ACID SEQUENCE OF A FUSION PROTEIN
COMPRISING IFNGR1-Fc-LINKER-BCMA
EMGTADLGPSSVPTPTNVTIESYNMNPIVYWEYQIMPQVPVTVEVKNYGVKNSEWIDCI
NISHHYCNISDHVGDPSNSLWVRVKARVGQKESAYAKSEEFAVCRDGKIGPPKLDIRKE
EKQIMIDIFHPSVFVNGDEQEVDYDPETTCYIRVYNVYVRMNGSEIQYKILTQKEDDCDE
IQCQLAIPVSSLNSQYCVSAEGVLHVWGVTTEKSKEVCITIFNSSIKG
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
SEQ ID NO: 153 - AMINO ACID SEQUENCE OF A FUSION PROTEIN
COMPRISING ICOSR-Fc-LINKER-BCMA-LINKER-BAFFR
EINGSANYEMFIFHNGGVQILCKYPDIVQQFKMQLLKGGQILCDLTKTKGSGNTVSIKSL
KFCHSQLSNNSVSFFLYNLDHSHANYYFCNLSIFDPPPFKVTLTGGYLHIYESQLCCQLK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
LQMAGQCSQNEYFDSLLHACIPCQLRCSSNTPPLTCQRYCNASVTNSVKGTNA
GGGGSGGGS
RGPRSLRGRDAPAPTPCVPAECFDLLVRHCVACGLLRTPRPKPAGASSPAPRTALQPQES
VGAGAGEAALPLPGLL
SEQ ID NO: 154 - AMINO ACID SEQUENCE OF A FUSION PROTEIN
COMPRISING IFNAR2 (high affinity)-LINKER-BAFFR
ISYDSPDYTDESCTFKISLRNFRSILSWELKNHSIVPTHYTLLYTIMSKPEDLKVVKNCA
NTTRSFCDLTDEWRSTHEAYVTVLEGFSGNTTLFSCSHNFWLAIDMSFEPPEFEIVGFTN
HINVMVKFPSIVEEELQFDLSL VIEEQSEGIVKKHKPEIKGNMSGNFTYIIDKLIPNTNY
CVSVYLEHSDEQAVIKSPLKCTLLPPGQESESAESAK
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 155 - AMINO ACID SEQUENCE OF A FUSION PROTEIN
COMPRISING IL21R-LINKER-GAMMACHAIN-FC-LINKER-BAFFR
CPDLVCYTDYLQTVICILEMWNLHPSTLTLTWQDQYEELKDEATSCSLHRSAHNATHAT
YTCHMDVFHFMADDIFSVNITDQSGNYSQECGSFLLAESIKPAPPFNVTVTFSGQYNISW
RSDYEDPAFYMLKGKLQYELQYRNRGDPWAVSPRRKLISVDSRSVSLLPLEFRKDSSYE
LQVRAGPMPGSSYQGTWSEWSDPVIFQTQSEELKE
GGGGSGGGGSGGGS
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
ATQMLKLQNLVIPWAPENLTLHKLSERQLELNWNNRFLNHCLEHLVQYRTDWDHSWT
EQSVDYRHKFSLPSVDGQKRYTFR VRSRFNPLCGSAQHWSEWSHPIHWGSNTSKENPFL
FALEA
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 156 - AMINO ACID SEQUENCE OF A FUSION PROTEIN
COMPRISING IL-17 RA-Fc/IgG1-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
EPKSSDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 157 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCP APELLGGPSV FLFPPKPKDT LMISRTPEVT CVVVDVSHED
PEVKFNWYVDGVEVHNAKTK PREEQYNSTY RVVSVLTVLH QDWLNGKEYK
CKVSNKALPA PIEKTISKAKGQPREPQVYT LPPSRDELTK NQVSLTCLVK
GFYPSDIAVE WESNGQPENN YKTTPPVLDS DGSFFLYSKL TVDKSRWQQG
NVFSCSVLHE ALHSHYTQKS LSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 158 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCP APELLGGPDV FLFPPKPKDT LMISRTPEVT 1DVSHED PEVKFNWYVD
GVEVHNAKTK PREEQYNSTY RVVSVLTVLH QDWLNGKEYK CKVSNKALPA
PEEKTISKAKGQPREPQVYT LPPSRDELTK NQVSLTCLVK GFYPSDIAVE
WESNGQPENN YKTTPPVLDS DGSFFLYSKL TVDKSRWQQG NVFSCSVMHE
ALHNHYTQKS LSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 159 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPEAAGGPSVFLFPPKPKDTLMISRTPEVTCVVVDVSHEDPEVKFNWYV
D
GVEVHNAKTKPREEQYNSTYRVVSVLTVLHQDWLNGKEYKCKVSNKALPAPIEKTISK
AK
GQPREPQVYTLPPSRDELTKNQVSLTCLVKGFYPSDIAVEWESNGQPENNYKTTPPVLD
S
DGSFFLYSKLTVDKSRWQQGNVFSCSVMHEALHNHYTQKSLSLSPGK
LGGGGSGGGGSGGGGSAAA
RGPRSLRGRDAPAPTPCVPAECFDLLVRHCVACGLLRTPRPKPAGASSPAPRTALQPQES
VGAGAGEAALPLPGLL
SEQ ID NO: 160 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
DGVEVHAAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYT
QKSLSLSPGK
GGGGSRGPRSLRGRDAPAPTPCVPAECFDLLVRHCVACGLLRTPRPKPAGASSPAPRTA
LQPQESVGAGAGEAALPLPGLL
SEQ ID NO: 161 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPSVFLFPPKPKDTLMISRTPEVTCVVVDVSHEDPEVKFNWYV
D
GVEVHAAKTKPREEQYNSTYRVVSVLTVLH
QDWLNGKEYKCKVSNKALPAPIEKTISKAK
GQPREPQVYTLPPSRDELTKNQVSLTCLVKGFYPSDIAVEWESNGQPENNYKTTPPVLD
S
DGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 162 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
DKTHTCPPCPAPELLGGPDVFLFPPKPKDTLMISRTPEVT
CVVVDVSHEDPEVKFNWYVD
GVEVHNAKTK PREEQYNSTYRVVSVLTVLH QDWLNGKEYK
CKVSNKALPAPEEKTISKAK
GQPREPQVYTLPPSRDELTKNQVSLTCLVK GFYPSDIAVE
WESNGQPENNYKTTPPVLDS
DGSFFLYSKLTVDKSRWQQG NVFSCSVMHE ALHNHYTQKS LSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 163 - AMINO ACID SEQUENCE OF A FUSION PROTEIN
COMPRISING IL-17 RA-Fc/IgG1-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
EPKSSDKTHTCPSCSAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 164 - AMINO ACID SEQUENCE OF A FUSION PROTEIN
COMPRISING IL-17 RA-Fc/IgG4-LINKER-BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
KYGPPCPPCPAPEFLGGPSVFLFPPKPKDTLMISRTPEVTCVVVDVSQED
PEVQFNWYVD GVEVHNAKTK PREEQFNSTYRVVSVLTVLH
QDWLNGKEYKCKVSNKGLPSSIEKTISKAK
GQPREPQVYTLPPSQEEMTKNQVSLTCLVK
GFYPSDIAVEWESNGQPENNYKTTPPVLDS
DGSFFLYSRLTVDKSRWQEGNVFSCSVMHEALHNHYTQKSLSLSLGK
GGGGS
RGPRSLRGRDAPAPTPCVPAECFDLLVRHCVACGLLRTPRPKPAGASSPAPRTALQPQES
VGAGAGEAALPLPGLL
SEQ ID NO: 165 - AMINO ACID SEQUENCE OF A FUSION PROTEIN
COMPRISING BCMA-LINKER-BCMA-LINKER-BCMA-Fc/IgG4-LINKER-
BAFFR-LINKER-BAFFR-LINKER-BAFFR
LQMAGQCSQNEYFDSLLHACIPCQLRCSSNTPPLTCQRYCNASVTNSVKGTNAG
GGGSLVPRGSGGGG
LQMAGQCSQNEYFDSLLHACIPCQLRCSSNTPPLTCQRYCNASVINSVKGTNAG
GGGSGGGGS
LQMAGQCSQNEYFDSLLHACIPCQLRCSSNTPPLTCQRYCNASVTNSVKGTNA
DHKPSNTKVDKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNH
YTQKSLSLSLGK
GGGGSGGGGSRGPRSLRGRDAPAPTPCVPAECFDLLVRHCVACGLLRTPRPKPAGASSP
APRTALQPQESVGAGAGEAALPLPGLLGGGGSGGGGSRGPRSLRGRDAPAPTPCVPAEC
FDLLVRHCVACGLLRTPRPKPAGASSPAPRTALQPQESVGAGAGEAALPLPGLLGGGGS
GGGGSRGPRSLRGRDAPAPTPCVPAECFDLLVRHCVACGLLRTPRPKPAGASSPAPRTA
LQPQESVGAGAGEAALPLPGLL
SEQ ID NO: 166 - AMINO ACID SEQUENCE OF A FUSION PROTEIN
COMPRISING BCMA-LINKER-BCMA-Fc/IgG1-BAFFR-LINKER-BAFFR-
LINKER-BAFFR
AQQCFHSEYFDSLLHACKPCHLRCSNPPATCQPYCDPSVTSSVKGTYT
GGGGSLVPRGSGGGG
AQQCFHSEYFDSLLHACKPCHLRCSNPPATCQPYCDPSVTSSVKGTYTGGGGSL
VPRGSGGGG
AQQCFHSEYFDSLLHACKPCHLRCSNPPATCQPYCDPSVTSSVKGTYT
DKTHTCPPCP APELLGGPDV FLFPPKPKDT LMISRTPEVT CVVVDVSHED
PEVKFNWYVDGVEVHNAKTK PREEQYNSTY RVVSVLTVLH QDWLNGKEYK
CKVSNKALPA PEEKTISKAKGQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK GGGGSGGGGS
RGPRSLRGRDAPAPTPCVPAECFDLLVRHCVACGLLRTPRPKPAGASSPAPRTALQPQES
VGAGAGEAALPLPGLLGGGGSGGGGSRGPRSLRGRDAPAPTPCVPAECFDLLVRHCVA
CGLLRTPRPKPAGASSPAPRTALQPQESVGAGAGEAALPLPGLLGGGGSGGGGSRGPRS
LRGRDAPAPTPCVPAECFDLLVRHCVACGLLRTPRPKPAGASSPAPRTALQPQESVGAG
AGEAALPLPGLL
SEQ ID NO: 167 - AMINO ACID SEQUENCE OF A FUSION PROTEIN
COMPRISING CD40-Fc-LINKER-BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLL
SEQ ID NO: 168 - AMINO ACID SEQUENCE OF A FUSION PROTEIN
COMPRISING CD40-Fc-LINKER-BAFFR-LINKER-BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLL
GGGGSGGGGSRGPRSLRGRDAPAPTPCVPAECFDLLVRHCVACGLLRTPRPKPAGASSP
APRTALQPQESVGAGAGEAALPLPGLL
SEQ ID NO: 169 - AMINO ACID SEQUENCE OF A FUSION PROTEIN
COMPRISING CD40-Fc-LINKER-BAFFR-LINKER-BAFFR-LINKER-BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLL
GGGGSGGGGSRGPRSLRGRDAPAPTPCVPAECFDLLVRHCVACGLLRTPRPKPAGASSP
APRTALQPQESVGAGAGEAALPLPGLL
GGGGSGGGGSRGPRSLRGRDAPAPTPCVPAECFDLLVRHCVACGLLRTPRPKPAGASSP
APRTALQPQESVGAGAGEAALPLPGLL
SEQ ID NO: 170 - AMINO ACID SEQUENCE OF A FUSION PROTEIN
COMPRISING CD40-Fc-LINKER-BAFFR-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGS
RGPRSLRGRDAPAPTPCVPAECFDLLVRHCVACGLLRTPRPKPAGASSPAPRTALQPQES
VGAGAGEAALPLPGLLLAEAAAKEAAAKEAAAKELQMAGQCSQNEYFDSLLHACIPC
QLRCSSNTPPLTCQRYCNASVTNSVKGTNA
SEQ ID NO: 171 - AMINO ACID SEQUENCE OF A FUSION PROTEIN
COMPRISING CD40-Fc-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKE
LQMAGQCSQNEYFDSLLHACIPCQLRCSSNTPPLTCQRYCNASVTNSVKGTNA
SEQ ID NO: 172 - AMINO ACID SEQUENCE OF A FUSION PROTEIN
COMPRISING CD40-Fc-LINKER-BCMA-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
LAEAAAKEAAAKEAAAKELQMAGQCSQNEYFDSLLHACIPCQLRCSSNTPPLTCQRYC
NASVTNSVKGTNA
GGGGSGGGGSLQMAGQCSQNEYFDSLLHACIPCQLRCSSNTPPLTCQRYCNASVTNSV
KGTNA
SEQ ID NO: 173 - AMINO ACID SEQUENCE OF A FUSION PROTEIN
COMPRISING CD40-Fc-LINKER-BCMA-LINKER-BCMA-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSGGGGSLQMAGQCSQNEYFDSLLHACIPCQLRCSSNTPPLTCQRYCNASVTNSV
KGTNA
GGGGSGGGGSLQMAGQCSQNEYFDSLLHACIPCQLRCSSNTPPLTCQRYCNASVINSV
KGTNA
GGGGSGGGGSLQMAGQCSQNEYFDSLLHACIPCQLRCSSNTPPLTCQRYCNASVINSV
KGTNA
SEQ ID NO: 174 - AMINO ACID SEQUENCE OF A FUSION PROTEIN
COMPRISING CD40-Fc-LINKER-BAFFR-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSRGPRSLRGRDAPAPTPCVPAECFDLLVRHCVACGLLRTPRPKPAGASSPAPRTA
LQPQESVGAGAGEAALPLPGLL
GGGGSLQMAGQCSQNEYFDSLLHACIPCQLRCSSNTPPLTCQRYCNASVTNSVKGTNA
SEQ ID NO: 175 - AMINO ACID SEQUENCE OF A FUSION PROTEIN
COMPRISING CD40-Fc-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSLQMAGQCSQNEYFDSLLHACIPCQLRCSSNTPPLTCQRYCNASVTNSVKGTNA
SEQ ID NO: 176 - AMINO ACID SEQUENCE OF A FUSION PROTEIN
COMPRISING CD40-Fc-LINKER-BCMA-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSLQMAGQCSQNEYFDSLLHACIPCQLRCSSNTPPLTCQRYCNASVTNSVKGTNA
GGGGSLQMAGQCSQNEYFDSLLHACIPCQLRCSSNTPPLTCQRYCNASVTNSVKGTNA
SEQ ID NO: 177 - AMINO ACID SEQUENCE OF A FUSION PROTEIN
COMPRISING CD40-Fc-LINKER-BCMA-LINKER-BCMA-LINKER-BCMA
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
GGGGSLQMAGQCSQNEYFDSLLHACIPCQLRCSSNTPPLTCQRYCNASVTNSVKGTNA
GGGGSLQMAGQCSQNEYFDSLLHACIPCQLRCSSNTPPLTCQRYCNASVTNSVKGTNA
GGGGSLQMAGQCSQNEYFDSLLHACIPCQLRCSSNTPPLTCQRYCNASVTNSVKGTNA
SEQ ID NO: 178 - AMINO ACID SEQUENCE OF A FUSION PROTEIN
COMPRISING COMMON GAMMA CHAIN and IL21R-LINKER-Fc-LINKER-
BAFFR
LNTTILTPNGNEDTTADFFLTTMPTDSLSVSTLPLPEVQCFVFNVEYMNCTWNSSSEPQP
TNLTLHYWYKNSDNDKVQKCSHYLFSEEITSGCQLQKKEIHLYQTFVVQLQDPREPRRQ
A
TQMLKLQNLVIPWAPENLTLHKLSESQLELNWNNRFLNHCLEHLVQYRTDWDHSWTE
QSV
DYRHKFSLPSVDGQKRYTFRVRSRFNPLCGSAQHWSEWSHPIHWGSNTSKENPFLFALE
A
GGGGSGGGGSGGGGSCPDLVCYTDYLQTVICILEMWNLHPSTLTLTWQDQYEELKDEA
TSCSLHRSAHNATHATYTCHMDVFHFMADDIFSVNITDQSGNYSQECGSFLLAESIKPAP
PFNVTVTFSGQYNISWRSDYEDPAFYMLKGKLQYELQYRNRGDPWAVSPRRKLISVDS
RSVSLLPLEFRKDSSYELQ
VRAGPMPGSSYQGTWSEWSDPVIFQTQSEELKEDKTHTCPPCPAPELLGGPSVFLFPPKP
KDTLMISRTPEVTCVVVD VSHEDPEVKF NWYVDGVEVH NAKTKPREEQ
YNSTYRVVSV LTVLHQDWLNGKEYKCKVSN KALPAPIEKT ISKAKGQPRE
PQVYTLPPSR DELTKNQVSL TCLVKGFYPSDIAVEWESNG QPENNYKTTP
PVLDSDGSFF LYSKLTVDKS RWQQGNVFSC
SVMHEALHNHYTQKSLSLSPGKGGGGSGGGGS
RGPRSLRG RDAPAPTPCV PAECFDLLVR HCVACGLLRT PRPKPAGASS PAPRTALQPQ
ESVGAGAGEA
SEQ ID NO: 179 - AMINO ACID SEQUENCE OF A FUSION PROTEIN
COMPRISING IL23R-LINKER-IL12 R Beta-1-Fc-BAFFR-LINKER-BAFFR
GITNINCSGHIWVEPATIFKMGMNISIYCQAAIKNCQPRKLHFYKNGIKERFQITRINKT
TARLWYKNFLEPHASMYCTAECPKHFQETLICGKDISSGYPPDIPDEVTCVIYEYSGNMT
CTWNAGKLTYIDTKYVVHVKSLETEEEQQYLTSSYINISTDSLQGGKKYLVWVQAANA
LGMEESKQLQIHLDDIVIPSAAVISRAETINATVPKTIIYWDSQTTIEKVSCEMRYKATTN
QTWNVKEFDTNFTYVQQSEFYLEPNIKYVFQVRCQETGKRYWQPWSSLFFHKTPETVP
QVTSKAFQHDTWNSGLTVASISTGHLTSDNRGDIG
LAEAAAKEAAAKEAAAKEAAAKAAA
CRTSECCFQDPPYPDADSGSASGPRDLRCYRISSDRYECSWQYEGPTAGVSHFLRCCLSS
GRCCYFAAGSATRLQFSDQAGVSVLYTVTLWVESWARNQTEKSPEVTLQLYNSVKYEP
PLGDIKVSKLAGQLRMEWETPDNQVGAEVQFRHRTPSSPWKLGDCGPQDDDTESCLCP
LEMNVAQEFQLRRRQLGSQGSSWSKWSSPVCVPPENPPQPQVRFSVEQLGQDGRRRLT
LKEQPTQLELPEGCQGLAPGTEVTYRLQLHMLSCPCKAKATRTLHLGKMPYLSGAAYN
VAVISSNQFGPGLNQTWHIPADTHTEPVALNISVGTNGTTMYWPARAQSMTYCIEWQP
VGQDGGLATCSLTAPQDPDPAGMATYSWSRESGAMGQEKCYYITIFASAHPEKLTLWS
TVLSTYHFGGNASAAGTPHHVSVKNHSLDSVSVDWAPSLLSTCPGVLKEYVVRCRDED
SKQVSEHPVQPTETQVTLSGLRAGVAYTVQVRADTAWLRGVWSQPQRFSIEVQVSD
DKTHT CPPCPAPELL GGPSVFLFPP KPKDTLMISRTPEVTCVVVD VSHEDPEVKF
NWYVDGVEVH NAKTKPREEQ YNSTYRVVSV LTVLHQDWLNGKEYKCKVSN
KALPAPIEKT ISKAKGQPRE PQVYTLPPSR DELTKNQVSL TCLVKGFYPS
DIAVEWESNG QPENNYKTTP PVLDSDGSFF LYSKLTVDKS RWQQGNVFSC
SVMHEALHNHYTQKSLSLSPGKGGGGSGGGGS
RGPRSLRGRDAPAPTPCVPAECFDLLVRHCVACGLLRTPRPKPAGASSPAPRTALQPQES
VGAGAGEAALPLPGLLGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 180 OF A FUSION PROTEIN COMPRISING EXTRACELLULAR
DOMAINS OF SOLUBLE PD-1 RECEPTOR -FCIGG4 -LINKER
ALFIBERCEPT
FLDSPDRPWNPPTFSPALLVVTEGDNATFTCSFSNTSESFVLNWYRMSPSNQTDKLAAFP
EDRSQPGQDCRFRVTQLPNGRDFHMSVVRARRNDSGTYLCGAISLAPKAQIKESLRAEL
R
VTERRAEVPTAHPSPSPRPAGQFQTLVDKTHTCPPCPAPEAAGGPSV FLFPPKPKDT
NVDHKPS NTKVDKRVES KYGPPCPPCP APEFLGGPSV
FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT
LPPSQEEMTK
NQVSLTCLVK GFYPSDIAVE WESNGQPENN YKTTPPVLDS DGSFFLYSRL
TVDKSRWQEG
NVFSCSVMHE ALHNHYTQKS LSLSLGKGGGGSGGGGS
SDTGRPFVEM YSEIPEIIHM TEGRELVIPC RVTSPNITVT LKKFPLDTLI PDGKRIIWDS
RKGFIISNAT YKEIGLLTCE ATVNGHLYKT NYLTHRQTNT IIDVVLSPSH
GIELSVGEKL
VLNCTARTEL NVGIDENWEY PSSKHQHKKL VNRDLKTQSG SEMKKFLSTL
TIDGVTRSDQ
GLYTCAASSG LMTKKNSTFV RVHEK
SEQ ID NO: 181 - AMINO ACID SEQUENCE OF A FUSION PROTEIN
COMPRISING AN EXTRACELLULAR DOMAIN OF BDCA2-FCIGG4-
LINKER- RECOMBINANT BAFFR
HNFMYSKTVKRLSKLREYQQYHPSLTCVMEGKDIEDWSCCPTPWTSFQSSCYFISTG
MQSWTKSQKNCSVMGADLVVINTREEQDFIIQNLKRNSSYFLGLSDPGGRRHWQWV
DQTPYNENVTFWHSGEPNNLDERCAIINFRSSEEWGWNDIHCHVPQKSICKMKKIYI
DHKPS NTKVDKRVES KYGPPCPPCP APEFLGGPSV
FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT
LPPSQEEMTK
NQVSLTCLVK GFYPSDIAVE WESNGQPENN YKTTPPVLDS DGSFFLYSRL
TVDKSRWQEG
NVFSCSVMHE ALHNHYTQKS LSLSLGK
GGGGSGGGGSGGGGS
RGPRSLRG RDAPAPTPCV PAECFDLLVR HCVACGLLRT PRPKPAGASS
PAPRTALQPQ ESVGAGAGEA ALPLPG
SEQ ID NO: 182 - AMINO ACID SEQUENCE OF A FUSION PROTEIN
COMPRISING AN EXTRACELLULAR DOMAIN OF BDCA2-FCIGG4-
LINKER- RECOMBINANT CD40
HNFMYSKTVKRLSKLREYQQYHPSLTCVMEGKDIEDWSCCPTPWTSFQSSCYFISTGMQ
SWTKSQKNCSVMGADLVVINTREEQDFIIQNLKRNSSYFLGLSDPGGRRHWQWVDQTP
YNENVTFWHSGEPNNLDERCAIINFRSSEEWGWNDIHCHVPQKSICKMKKIYI
DHKPS NTKVDKRVES KYGPPCPPCP APEFLGGPSV
FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT
LPPSQEEMTK
NQVSLTCLVK GFYPSDIAVE WESNGQPENN YKTTPPVLDS DGSFFLYSRL
TVDKSRWQEG
NVFSCSVMHE ALHNHYTQKS LSLSLGK
GGGGSGGGGSGGGGS
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
ANTIBODY- SOLUBLE RECEPTOR FUSION PROTEINS and ANTIBODY-
CYTOKINE FUSIONS
SEQ ID NOS: 183-184 - AMINO ACID SEQUENCES OF A FUSION PROTEIN
COMPRISING SECUKINUMAB (COSENTYX)-LINKER-BAFFR-LINKER-
BAFFR
SEQ ID NO: 183 - HEAVY CHAIN FUSION
EVQLVESGGG LVQPGGSLRL SCAASGFTFS NYWMNWVRQA PGKGLEWVAA
INQDGSEKYYVGSVKGRFTI SRDNAKNSLY LQMNSLRVED TAVYYCVRDY
YDILTDYYIH YWYFDLWGRGTLVTVSSAST KGPSVFPLAP SSKSTSGGTA
ALGCLVKDYF PEPVTVSWNS GALTSGVHTFPAVLQSSGLY SLSSVVTVPS
SSLGTQTYIC NVNHKPSNTK VDKRVEPKSC DKTHTCPPCPAPELLGGPSV
FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK
GQPREPQVYTLPPSREEMTK NQVSLTCLVK GFYPSDIAVE WESNGQPENN
YKTTPPVLDS DGSFFLYSKLTVDKSRWQQG NVFSCSVMHE ALHNHYTQKS L
SLSPGK GGGGGGGGS
RGPRSLRGRDAPAPTPCVPAECFDLLVRHCVACGLLRTPRPKPAGASSPAPRTALQPQES
VGAGAGEAALPLPGLLGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 184 - LIGHT CHAIN
EIVLTQSPGT LSLSPGERAT LSCRASQSVS SSYLAWYQQK PGQAPRLLIY
GASSRATGIPDRFSGSGSGT DFTLTISRLE PEDFAVYYCQ QYGSSPCTFG QGTRLEIKRT
VAAPSVFIFPPSDEQLKSGT ASVVCLLNNF YPREAKVQWK VDNALQSGNS
QESVTEQDSK DSTYSLSSTLTLSKADYEKH KVYACEVTHQ GLSSPVTKSF NRGEC
SEQ ID NOS: 185-186 - AMINO ACID SEQUENCES OF A FUSION PROTEIN
COMPRISING SECUKINUMAB (COSENTYX)-LINKER-BCMA LINKER-
BCMA -LINKER-BCMA
SEQ ID NO: 185 - HEAVY CHAIN FUSION
EVQLVESGGG LVQPGGSLRL SCAASGFTFS NYWMNWVRQA PGKGLEWVAA
INQDGSEKYYVGSVKGRFTI SRDNAKNSLY LQMNSLRVED TAVYYCVRDY
YDILTDYYIH YWYFDLWGRGTLVTVSSAST KGPSVFPLAP SSKSTSGGTA
ALGCLVKDYF PEPVTVSWNS GALTSGVHTFPAVLQSSGLY SLSSVVTVPS
SSLGTQTYIC NVNHKPSNTK VDKRVEPKSC DKTHTCPPCPAPELLGGPSV
FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK
GQPREPQVYTLPPSREEMTK NQVSLTCLVK GFYPSDIAVE WESNGQPENN
YKTTPPVLDS DGSFFLYSKLTVDKSRWQQG NVFSCSVMHE ALHNHYTQKS L
SLSPGK GGGGSGGGGS
LQMAGQCSQNEYFDSLLHACIPCQLRCSSNTPPLTCQRYCNASVTNSVKGTNAGGGGS
GGGGS LQMAGQCSQNEYFDSLLHACIPCQLRCSSNTPPLTCQRYCNASVTNSVKGTNA
GGGGSGGGGSLQMAGQCSQNEYFDSLLHACIPCQLRCSSNTPPLTCQRYCNASVINSV
KGTNA
SEQ ID NO: 186 - LIGHT CHAIN
EIVLTQSPGT LSLSPGERAT LSCRASQSVS SSYLAWYQQK PGQAPRLLIY
GASSRATGIPDRFSGSGSGT DFTLTISRLE PEDFAVYYCQ QYGSSPCTFG QGTRLEIKRT
VAAPSVFIFPPSDEQLKSGT ASVVCLLNNF YPREAKVQWK VDNALQSGNS
QESVTEQDSK DSTYSLSSTLTLSKADYEKH KVYACEVTHQ GLSSPVTKSF NRGEC
SEQ ID NOS: 187 - 188 - AMINO ACID SEQUENCE OF A FUSION PROTEIN
COMPRISING ADALIMUMAB /HUMIRA-LINKER-TACI-LINKER-BAFF
SEQ ID NO: 187 - HEAVY CHAIN FUSION
EVQLVESGGGLVQPGRSLRLSCAASGFTFDDYAMHWVRQAPGKGLEWVSAITWNSGH
IDY
ADSVEGRFTISRDNAKNSLYLQMNSLRAEDTAVYYCAKVSYLSTASSLDYWGQGTLVT
VS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREE
QY
NSTYRVVSVLTVLHQDWLNGKEYKCKVSNKALPAPIEKTISKAKGQPREPQVYTLPPSR
D
ELTKNQVSLTCLVKGFYPSDIAVEWESNGQPENNYKTTPPVLDSDGSFFLYSKLTVDKS
R
WQQGNVFSCSVMHEALHNHYTQKSLSLSPGKGGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYSGGGGSGGGGSGG
GSRGPRSLRGRDAPAPTPCVPAECFDLLVRHCVACGLLRTPRPKPAGASSPAPRTALQPQ
ESVGAGAGEAALPLPGLL
SEQ ID NO: 188 - LIGHT CHAIN
DIQMTQSPSSLSASVGDRVTITCRASQGIRNYLAWYQQKPGKAPKLLIYAASTLQSGVPS
RFSGSGSGTDFTLTISSLQPEDVATYYCQRYNRAPYTFGQGTKVEIKRTVAAPSVFIFPP
SDEQLKSGTASVVCLLNNFYPREAKVQWKVDNALQSGNSQESVTEQDSKDSTYSLSST
LT
LSKADYEKHKVYACEVTHQGLSSPVTKSFNRGEC
SEQ ID NOS: 189 - 190 - AMINO ACID SEQUENCES OF A FUSION PROTEIN
COMPRISING PEMOBROLIZUMAB-LINKER-SOLUBLE RECOMBINANT
VEGFR
SEQ ID NO: 189 - HEAVY CHAIN FUSION
QVQLVQSGVE VKKPGASVKV SCKASGYTFT NYYMYWVRQA PGQGLEWMGG
INPSNGGTNF
NEKFKNRVTL TTDSSTTTAY MELKSLQFDD TAVYYCARRD YRFDMGFDYW
GQGTTVTVSS
ASTKGPSVFP LAPCSRSTSE STAALGCLVK DYFPEPVTVS WNSGALTSGV
HTFPAVLQSS
GLYSLSSVVT VPSSSLGTKT YTCNVDHKPS NTKVDKRVES KYGPPCPPCP
APEFLGGPSV
FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT
LPPSQEEMTK
NQVSLTCLVK GFYPSDIAVE WESNGQPENN YKTTPPVLDS DGSFFLYSRL
TVDKSRWQEG
NVFSCSVMHE ALHNHYTQKS LSLSLGK YKTTPPVLDS DGSFFLYSRL TVDKSRWQEG
NVFSCSVMHE ALHNHYTQKS LSLSLGKGGGGSGGGGSGGGGS
SDTGRPFVEM YSEIPEIIHM TEGRELVIPC RVTSPNITVT LKKFPLDTLI PDGKRIIWDS
RKGFIISNAT YKEIGLLTCE ATVNGHLYKT NYLTHRQTNT IIDVVLSPSH
GIELSVGEKL
VLNCTARTEL NVGIDENWEY PSSKHQHKKL VNRDLKTQSG SEMKKFLSTL
TIDGVTRSDQ
GLYTCAASSG LMTKKNSTFV RVHEK
SEQ ID NO: 190 - LIGHT CHAIN
EIVLTQSPAT LSLSPGERAT LSCRASKGVS TSGYSYLHWY QQKPGQAPRL
LIYLASYLES GVPARFSGSG SGTDFTLTIS SLEPEDFAVY YCQHSRDLPL
TFGGGTKVEI KRTVAAPSVFIFPPSDEQLK SGTASVVCLL NNFYPREAKV
QWKVDNALQS GNSQESVTEQ DSKDSTYSLSSTLTLSKADY EKHKVYACEV
THQGLSSPVT KSFNRGEC
SEQ ID NOS: 191-194 AMINO ACID SEQUENCESOF A FUSION PROTEIN
COMPRISING MOSUNETUZUMAB, A BISPECIFIC CD20/CD3 ANTIBODY-
LINKER-BAFFR-LINKER-BAFFR
SEQ ID NO: 191 - A CHAIN FUSION
EVQLVQSGAE VKKPGASVKV SCKASGYTFT NYYIHWVRQA PGQGLEWIGW
IYPGDGNTKY
NEKFKGRATL TADTSTSTAY LELSSLRSED TAVYYCARDS YSNYYFDYWG
QGTLVTVSSA
STKGPSVFPL APSSKSTSGG TAALGCLVKD YFPEPVTVSW NSGALTSGVH
TFPAVLQSSG
LYSLSSVVTV PSSSLGTQTY ICNVNHKPSN TKVDKKVEPK SCDKTHTCPP
CPAPELLGGP
SVFLFPPKPK DTLMISRTPE VTCVVVDVSH EDPEVKFNWY VDGVEVHNAK
TKPREEQYGS
TYRVVSVLTV LHQDWLNGKE YKCKVSNKAL PAPIEKTISK AKGQPREPQV
YTLPPSREEM
TKNQVSLSCA VKGFYPSDIA VEWESNGQPE NNYKTTPPVL DSDGSFFLVS
KLTVDKSRWQ
QGNVFSCSVM HEALHNHYTQ KSLSLSPGK
GGGGSGGGGSGGGGSRGPRSLRGRDAPAPTPCVPAECFDLLVRHCVACGLLRTPRPKP
AGASSPAPRTALQPQESVGAGAGEAALPLPGLLGGGGSGGGGSGGGGSRGPRSLRGRD
APAPTPCVPAECFDLLVRHCVACGLLRTPRPKPAGASSPAPRTALQPQESVGAGAGEAA
LPLPGLL
SEQ ID NO: 192 - B CHAIN FUSION
EVQLVESGGG LVQPGGSLRL SCAASGYTFT SYNMHWVRQA PGKGLEWVGA
IYPGNGDTSY
NQKFKGRFTI SVDKSKNTLY LQMNSLRAED TAVYYCARVV YYSNSYWYFD
VWGQGTLVTV
SSASTKGPSV FPLAPSSKST SGGTAALGCL VKDYFPEPVT VSWNSGALTS
GVHTFPAVLQ
SSGLYSLSSV VTVPSSSLGT QTYICNVNHK PSNTKVDKKV EPKSCDKTHT
CPPCPAPELL
GGPSVFLFPP KPKDTLMISR TPEVTCVVVD VSHEDPEVKF NWYVDGVEVH
NAKTKPREEQ
YGSTYRVVSV LTVLHQDWLN GKEYKCKVSN KALPAPIEKT ISKAKGQPRE
PQVYTLPPSR
EEMTKNQVSL WCLVKGFYPS DIAVEWESNG QPENNYKTTP PVLDSDGSFF
LYSKLTVDKS
RWQQGNVFSC SVMHEALHNH YTQKSLSLSP GK
GGGGSGGGGSGGGGSRGPRSLRGRDAPAPTPCVPAECFDLLVRHCVACGLLRTPRPKP
AGASSPAPRTALQPQESVGAGAGEAALPLPGLLGGGGSGGGGSGGGGSRGPRSLRGRD
APAPTPCVPAECFDLLVRHCVACGLLRTPRPKPAGASSPAPRTALQPQESVGAGAGEAA
LPLPGLL
SEQ ID NO: 193 - LIGHT CHAIN A
DIVMTQSPDS LAVSLGERAT INCKSSQSLL NSRTRKNYLA WYQQKPGQPP
KLLIYWASTR
ESGVPDRFSG SGSGTDFTLT ISSLQAEDVA VYYCTQSFIL RTFGQGTKVE
IKRTVAAPSV
FIFPPSDEQL KSGTASVVCL LNNFYPREAK VQWKVDNALQ SGNSQESVTE
QDSKDSTYSL
SSTLTLSKAD YEKHKVYACE VTHQGLSSPV TKSFNRGEC
SEQ ID NO: 194 - LIGHT CHAIN B
DIQMTQSPSS LSASVGDRVT ITCRASSSVS YMHWYQQKPG KAPKPLIYAP
SNLASGVPSR
FSGSGSGTDF TLTISSLQPE DFATYYCQQW SFNPPTFGQG TKVEIKRTVA APSVFIFPPS
DEQLKSGTAS VVCLLNNFYP REAKVQWKVD NALQSGNSQE SVTEQDSKDS
TYSLSSTLTL
SKADYEKHKV YACEVTHQGL SSPVTKSFNR GEC
SEQ ID NOS: 195-196 AMINO ACID SEQUENCES OF A FUSION PROTEIN
COMPRISING ANTI-IFN-TYPE 1 RECEPTOR ANTIBODY-LINKER-BAFFR-
LINKER-BAFFR-LINKER-BAFFR
SEQ ID NO: 195 - HEAVY CHAIN FUSION
EVQLVQSGAE VKKPGESLKI SCKGSGYIFT NYWIAWVRQM PGKGLESMGI
IYPGDSDIRY
SPSFQGQVTI SADKSITTAY LQWSSLKASD TAMYYCARHD IEGFDYWGRG
TLVTVSSAST
KGPSVFPLAP SSKSTSGGTA ALGCLVKDYF PEPVTVSWNS GALTSGVHTF
PAVLQSSGLY
SLSSVVTVPS SSLGTQTYIC NVNHKPSNTK VDKRVEPKSC DKTHTCPPCP
APEFEGGPSV
FLFPPKPKDT LMISRTPEVT CVVVDVSHED PEVKFNWYVD GVEVHNAKTK
PREEQYNSTY
RVVSVLTVLH QDWLNGKEYK CKVSNKALPA SIEKTISKAK GQPREPQVYT
LPPSREEMTK
NQVSLTCLVK GFYPSDIAVE WESNGQPENN YKTTPPVLDS DGSFFLYSKL
TVDKSRWQQG
NVFSCSVMHE ALHNHYTQKS LSLSPGK
GGGGSGGGGSGGGGSRGPRSLRGRDAPAPTPCVPAECFDLLVRHCVACGLLRTPRPKP
AGASSPAPRTALQPQESVGAGAGEAALPLPGLLGGGGSGGGGSGGGGSRGPRSLRGRD
APAPTPCVPAECFDLLVRHCVACGLLRTPRPKPAGASSPAPRTALQPQESVGAGAGEAA
LPLPGLL
GGGGSGGGGSGGGGSRGPRSLRGRDAPAPTPCVPAECFDLLVRHCVACGLLRTPRPKP
AGASSPAPRTALQPQESVGAGAGEAALPLPGLLGGGGSGGGGSGGGGSRGPRSLRGRD
APAPTPCVPAECFDLLVRHCVACGLLRTPRPKPAGASSPAPRTALQPQESVGAGAGEAA
LPLPGLL
SEQ ID NO: 196 - (LIGHT CHAIN)
EIVLTQSPGT LSLSPGERAT LSCRASQSVS SSFFAWYQQK PGQAPRLLIY
GASSRATGIP
DRLSGSGSGT DFTLTITRLE PEDFAVYYCQ QYDSSAITFG QGTRLEIKRT VAAPSVFIFP
PSDEQLKSGT ASVVCLLNNF YPREAKVQWK VDNALQSGNS QESVTEQDSK
DSTYSLSSTL
TLSKADYEKH KVYACEVTHQ GLSSPVTKSF NRGEC
SEQ ID NOS: 197-198 - AMINO ACID SEQUENCES OF A FUSION PROTEIN
COMPRISING RITUXIMAB (ANTI CD20)-LINKER-BAFFR-LINKER-BAFFR
SEQ ID NO: 197 - HEAVY CHAIN FUSION
QVQLQQPGAELVKPGASVKMSCKASGYTFTSYNMHWVKQTPGRGLEWIGAIYPGNGD
TSYNQKFKGKATLTADKSSSTAYMQLSSLTSEDSAVYYCARSTYYGGDWYFNVWGAG
TTVTVSAASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
GGGGSGGGGSRGPRSLRG RDAPAPTPCV
PAECFDLLVRHCVACGLLRTPRPKPAGASSPAPRTALQPQ
ESVGAGAGEAALPLPGGGGGSGGGGSRGPRSLRGRDAPAPTPCVPAECFDLLVRHCVA
CGLLRT PRPKPAGASS PAPRTALQPQ ESVGAGAGEA ALPLPG
SEQ ID NO: 198 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NOS: 199-200 - AMINO ACID SEQUENCES OF A FUSION PROTEIN
COMPRISING SARILUMAZB (ANTI IL-6R ALPHA)-LINKER-BAFFR-
LINKER-BAFFR
SEQ ID NO: 199 - HEAVY CHAIN FUSION
EVQLVESGGG LVQPGRSLRL SCAASRFTFD DYAMHWVRQA PGKGLEWVSG
ISWNSGRIGY
ADSVKGRFTI SRDNAENSLF LQMNGLRAED TALYYCAKGR DSFDIWGQGT
MVTVSSASTK
GPSVFPLAPS SKSTSGGTAA LGCLVKDYFP EPVTVSWNSG ALTSGVHTFP
AVLQSSGLYS
LSSVVTVPSS SLGTQTYICN VNHKPSNTKV DKKVEPKSCD KTHTCPPCPA
PELLGGPSVF
LFPPKPKDTL MISRTPEVTC VVVDVSHEDP EVKFNWYVDG VEVHNAKTKP
REEQYNSTYRVVSVLTVLHQ DWLNGKEYKC KVSNKALPAP IEKTISKAKG
QPREPQVTYL PPSRDELTKNQVSLTCLVKG FYPSDIAVEW ESNGQPENNY
KTTPPVLDSD GSFFLYSKLT VDKSRWQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLLGGGGSGGGGSGGGGSRGPRSLRGRDAPAPTP
CVPAECFDLLVRHCVACGLLRTPRPKPAGASSPAPRTALQPQESVGAGAGEAALPLPGL
L
SEQ ID NO: 200 - LIGHT CHAIN
DIQMTQSPSS VSASVGDRVT ITCRASQGIS SWLAWYQQKP GKAPKLLIYG
ASSLESGVPSRFSGSGSGTD FTLTISSLQP EDFASYYCQQ ANSFPYTFGQ GTKLEIKRTV
AAPSVFIFPPSDEQLKSGTA SVVCLLNNFY PREAKVQWKV DNALQSGNSQ
ESVTEQDSKD STYSLSSTLTLSKADYEKHK VYACEVTHQG LSSPVTKSFN RGEC
SEQ ID NO: 201-202 - AMINO ACID SEQUENCES OF A FUSION PROTEIN
COMPRISING TAFASITAMAB (ANTI-CD19)-LINKER-BAFFR-LINKER-
BAFFR
SEQ ID NO: 201 - HEAVY CHAIN FUSION
EVQLVESGGG LVKPGGSLKL SCAASGYTFT SYVMHWVRQA PGKGLEWIGY
INPYNDGTKYNEKFQGRVTI SSDKSISTAY MELSSLRSED TAMYYCARGT
YYYGTRVFDY WGQGTLVTVSSASTKGPSVF PLAPSSKSTS GGTAALGCLV
KDYFPEPVTV SWNSGALTSG VHTFPAVLQS
SGLYSLSSVV TVPSSSLGTQ TYICNVNHKP SNTKVDKKVE PKSCDKTHTC
PPCPAPELLGGPDVFLFPPK PKDTLMISRT PEVTCVVVDV SHEDPEVQFN
WYVDGVEVHN AKTKPREEQFNSTFRVVSVL TVVHQDWLNG KEYKCKVSNK
ALPAPEEKTI SKTKGQPREP QVYTLPPSREEMTKNQVSLT CLVKGFYPSD
IAVEWESNGQ PENNYKTTPP MLDSDGSFFL YSKLTVDKSR
WQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLLGGGGSGGGGSGGGGSRGPRSLRGRDAPAPTP
CVPAECFDLLVRHCVACGLLRTPRPKPAGASSPAPRTALQPQESVGAGAGEAALPLPGL
L
SEQ ID NO: 202 - LIGHT CHAIN
DIVMTQSPAT LSLSPGERAT LSCRSSKSLQ NVNGNTYLYW FQQKPGQSPQ
LLIYRMSNLNSGVPDRFSGS GSGTEFTLTI SSLEPEDFAV YYCMQHLEYP
ITFGAGTKLE IKRTVAAPSVFIFPPSDEQL KSGTASVVCL LNNFYPREAK
VQWKVDNALQ SGNSQESVTE QDSKDSTYSLSSTLTLSKAD YEKHKVYACE
VTHQGLSSPV TKSFNRGEC
SEQ ID NO: 203-204 - AMINO ACID SEQUENCES OF A FUSION PROTEIN
COMPRISING BELIMUMAB -LINKER-BAFFR-LINKER-BAFFR
SEQ ID NO: 203 - HEAVY CHAIN
QVQLQQSGAE VKKPGSSVRV SCKASGGTFN NNAINWVRQA PGQGLEWMGGI
IPMFGTAKY
SQNFQGRVAITADESTGTAS MELSSLRSED TAVYYCARSR DLLLFPHHAL
SPWGRGTMVT
VSSASTKGPS VFPLAPSSKS TSGGTAALGC LVKDYFPEPV TVS NSGALT
SGVHTFPAVL
QSSGLYSLSS VVTVPSSSLG TQTYICNVNH KPSNTKVDKK VEPKSCDKTH
TCPPCPAPEL
LGGPSVFLFP PKPKDTLMI S RTPEVTCVVV DVSHEDPEVK FNWYVDGVEV
HNAKTKPREE
QYNSTYRVVS VLTVLHQDWL NGKEYKCKVS NKALPAPIEK TI SKAKGQPR
EPQVYTLPPS
RDELTKNQVS LTCLVKGFYP SDIAVEWESN GQPENNYKTT PPVLDSDGSF
FLYSKLTVDK
SRWQQGNVFS CSVMHEALHN HYTQKSLSLS PGKGGGGGGGGS
RGPRSLRGRDAPAPTPCVPAECFDLLVRHCVACGLLRTPRPKPAGASSPAPRTALQPQ
ESVGAGAGEAALPLPGLL GGGGSGGGGS
RGPRSLRGRDAPAPTPCVPAECFDLLVRHCVACGLLRTPRPKPAGASSPAPRTALQPQ
ESVGAGAGEAALPLPGLL
SEQ ID NO: 204 - Light Chain
SSELTQDPAV SVALGQTVRV TCQGDSLRSY YASWYQQKPG QAPVLVIYGK NNRPSGI
PDR FSGSSSGNTA SLTITGAQAE DEADYYCSSR DSSGNHWVFG GGTELTVLGQ
PKAAPSVTLF PPSSEELQAN KATLVCLISD FYPGAVTVAW KADSSPV AG
VETTTPSKQS NNKYAASSYL SLTPEQWKSH RSYSCQVTHE GSTVEKTVAP TECS
SEQ ID NO: 205-206 - AMINO ACID SEQUENCES OF A FUSION PROTEIN
COMPRISING RITUXIMAB (ANTI CD20)-LINKER-BAFFR
SEQ ID NO: 205 - HEAVY CHAIN FUSION
QVQLQQPGAELVKPGASVKMSCKASGYTFTSYNMHWVKQTPGRGLEWIGAIYPGNGD
TSYNQKFKGKATLTADKSSSTAYMQLSSLTSEDSAVYYCARSTYYGGDWYFNVWGAG
TTVTVSAASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
GGGGSGGGGS
RGPRSLRG RDAPAPTPCV PAECFDLLVR HCVACGLLRT PRPKPAGASS PAPRTALQPQ
ESVGAGAGEA ALPLPG
SEQ ID NO: 206 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 207-208 - AMINO ACID SEQUENCES OF A FUSION PROTEIN
COMPRISING RITUXIMAB (ANTI CD20)-LINKER-BAFFR-LINKER-BAFFR-
LINKER-BAFFR
SEQ ID NO: 207 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGSRGPRSLRG RDAPAPTPCV PAECFDLLVR HCVACGLLRT
PRPKPAGASS PAPRTALQPQ ESVGAGAGEA ALPLPG GGGGSGGGGSRGPRSLRG
RDAPAPTPCV PAECFDLLVR HCVACGLLRT PRPKPAGASS PAPRTALQPQ
ESVGAGAGEA ALPLPG GGGGSGGGGS
RGPRSLRG RDAPAPTPCV PAECFDLLVR HCVACGLLRT PRPKPAGASS PAPRTALQPQ
ESVGAGAGEA ALPLPG
SEQ ID NO: 208 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 209-210 - AMINO ACID SEQUENCES OF A FUSION PROTEIN
COMPRISING RITUXIMAB (ANTI CD20)-LINKER-TACI
SEQ ID NO: 209 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 210 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 211-212 - AMINO ACID SEQUENCES OF A FUSION PROTEIN
COMPRISING RITUXIMAB (ANTI CD20)-LINKER-TACI-LINKER-TACI
SEQ ID NO: 211 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYSGGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 212 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 213-214 - AMINO ACID SEQUENCES OF A FUSION PROTEIN
COMPRISING RITUXIMAB (ANTI CD20)-LINKER-TACI-LINKER-TACI-
LINKER-TACI
SEQ ID NO: 213 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYSGGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 214 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 215-216 - AMINO ACID SEQUENCES OF A FUSION PROTEIN
COMPRISING RITUXIMAB (ANTI CD20)-LINKER-BCMA
SEQ ID NO: 215 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSSLQMAGQCS
QNEYFDSLLHACIPCQLRCSSNTPPLTCQRYCNASVTNSVKGTNA
SEQ ID NO: 216 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 217-218 - AMINO ACID SEQUENCES OF A FUSION PROTEIN
COMPRISING RITUXIMAB (ANTI CD20)-LINKER-BCMA-LINKER-BCMA
SEQ ID NO: 217 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGSSLQMAGQCSQNEYFDSLLHACIPCQLRCSSNTPPLTCQRYCNASVTNSV
KGTNA
GGGGSGGGGSSLQMAGQCSQNEYFDSLLHACIPCQLRCSSNTPPLTCQRYCNASVINSV
KGTNA
SEQ ID NO: 218 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 219-220 - AMINO ACID SEQUENCES OF A FUSION PROTEIN
COMPRISING RITUXIMAB (ANTI CD20)-LINKER-BCMA-LINKER-BCMA-
LINKER-BCMA
SEQ ID NO: 219 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGSSLQMAGQCSQNEYFDSLLHACIPCQLRCSSNTPPLTCQRYCNASVTNSV
KGTNAGGGGSGGGGSSLQMAGQCSQNEYFDSLLHACIPCQLRCSSNTPPLTCQRYCNA
SVTNSVKGTNAGGGGSGGGGSSLQMAGQCSQNEYFDSLLHACIPCQLRCSSNTPPLTCQ
RYCNASVTNSVKGTNA
SEQ ID NO: 220 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 221-222 - AMINO ACID SEQUENCES OF A FUSION PROTEIN
COMPRISING RITUXIMAB (ANTI CD20)-LINKER-BAFFR-LINKER-TACI
SEQ ID NO: 221 - HEAVY CHAIN FUSION
QKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
GGGGSGGGGSRGPRSLRGRDAPAPTPCVPAECFDLLVRHCVACGLLRTPRPKPAGASSP
APRTALQPQESVGAGAGEAALPLPGLL
GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 222 - LIGHT CHAIN
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NOS: 223-224 - AMINO ACID SEQUENCES OF A FUSION PROTEIN
COMPRISING BELIMUMAB-BAFFR
SEQ ID NO: 223 - HEAVY CHAIN
QVQLQQSGAE VKKPGSSVRV SCKASGGTFN NNAINWVRQA PGQGLEWMGG I
IPMFGTAKY
SQNFQGRVAI TADESTGTAS MELSSLRSED TAVYYCARSR DLLLFPHHAL
SPWGRGTMVT
VSSASTKGPS VFPLAPSSKS TSGGTAALGC LVKDYFPEPV TVS NSGALT
SGVHTFPAVL
QSSGLYSLSS VVTVPSSSLG TQTYICNVNH KPSNTKVDKK VEPKSCDKTH
TCPPCPAPEL
LGGPSVFLFP PKPKDTLMI S RTPEVTCVVV DVSHEDPEVK FNWYVDGVEV
HNAKTKPREE
QYNSTYRVVS VLTVLHQDWL NGKEYKCKVS NKALPAPIEK TI SKAKGQPR
EPQVYTLPPS
RDELTKNQVS LTCLVKGFYP SDIAVEWESN GQPENNYKTT PPVLDSDGSF
FLYSKLTVDK
SRWQQGNVFS CSVMHEALHN HYTOKSLSLS PGKGGGGSGGGGS
RGPRSLRGRDAPAPTPCVPAECFDLLVRHCVACGLLRTPRPKPAGASSPAPRTALQPQ
ESVGAGAGEAALPLPGLL
SEQ ID NO: 224 - LIGHT CHAIN
SSELTQDPAV SVALGQTVRV TCQGDSLRSY YASWYQQKPG QAPVLVIYGK NNRPSGI
PDR FSGSSSGNTA SLTITGAQAE DEADYYCSSR DSSGNHWVFG GGTELTVLGQ
PKAAPSVTLF PPSSEELQAN KATLVCLISD FYPGAVTVAW KADSSPV AG
VETTTPSKQS NNKYAASSYL SLTPEQWKSH RSYSCQVTHE GSTVEKTVAP TECS
SEQ ID NOS: 225-226 - AMINO ACID SEQUENCES OF A FUSION PROTEIN
COMPRISING BELIMUMAB-TACI
SEQ ID NO: 225 - HEAVY CHAIN
QVQLQQSGAE VKKPGSSVRV SCKASGGTFN NNAINWVRQA PGQGLEWMGG I
IPMFGTAKY
SQNFQGRVAI TADESTGTAS MELSSLRSED TAVYYCARSR DLLLFPHHAL
SPWGRGTMVT
VSSASTKGPS VFPLAPSSKS TSGGTAALGC LVKDYFPEPV TVS NSGALT
SGVHTFPAVL
QSSGLYSLSS VVTVPSSSLG TQTYICNVNH KPSNTKVDKK VEPKSCDKTH
TCPPCPAPEL
LGGPSVFLFP PKPKDTLMI S RTPEVTCVVV DVSHEDPEVK FNWYVDGVEV
HNAKTKPREE
QYNSTYRVVS VLTVLHQDWL NGKEYKCKVS NKALPAPIEK TI SKAKGQPR
EPQVYTLPPS
RDELTKNQVS LTCLVKGFYP SDIAVEWESN GQPENNYKTT PPVLDSDGSF
FLYSKLTVDK
SRWQQGNVFS CSVMHEALHN HYTQKSLSLS PGKGGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 226 - LIGHT CHAIN
SSELTQDPAV SVALGQTVRV TCQGDSLRSY YASWYQQKPG QAPVLVIYGK NNRPSGI
PDR FSGSSSGNTA SLTITGAQAE DEADYYCSSR DSSGNHWVFG GGTELTVLGQ
PKAAPSVTLF PPSSEELQAN KATLVCLISD FYPGAVTVAW KADSSPV AG
VETTTPSKQS NNKYAASSYL SLTPEQWKSH RSYSCQVTHE GSTVEKTVAP TECS
SEQ ID NOS: 227-228 - AMINO ACID SEQUENCES OF A FUSION PROTEIN
COMPRISING BELIMUMAB-BCMA
SEQ ID NO: 227 - HEAVY CHAIN
QVQLQQSGAE VKKPGSSVRV SCKASGGTFN NNAINWVRQA PGQGLEWMGGI
IPMFGTAKY
SQNFQGRVAI TADESTGTAS MELSSLRSED TAVYYCARSR DLLLFPHHAL
SPWGRGTMVT
VSSASTKGPS VFPLAPSSKS TSGGTAALGC LVKDYFPEPV TVS NSGALT
SGVHTFPAVL
QSSGLYSLSS VVTVPSSSLG TQTYICNVNH KPSNTKVDKK VEPKSCDKTH
TCPPCPAPEL
LGGPSVFLFP PKPKDTLMI S RTPEVTCVVV DVSHEDPEVK FNWYVDGVEV
HNAKTKPREE
QYNSTYRVVS VLTVLHQDWL NGKEYKCKVS NKALPAPIEK TI SKAKGQPR
EPQVYTLPPS
RDELTKNQVS LTCLVKGFYP SDIAVEWESN GQPENNYKTT PPVLDSDGSF
FLYSKLTVDK
SRWQQGNVFS CSVMHEALHN HYTQKSLSLS PGKGGGGSGGGGS
LQMAGQCSQNEYFDSLLHACIPCQLRCSSNTPPLTCQRYCNASVTNSVKGTNA
SEQ ID NO: 228 - LIGHT CHAIN
SSELTQDPAV SVALGQTVRV TCQGDSLRSY YASWYQQKPG QAPVLVIYGK NNRPSGI
PDR FSGSSSGNTA SLTITGAQAE DEADYYCSSR DSSGNHWVFG GGTELTVLGQ
PKAAPSVTLF PPSSEELQAN KATLVCLISD FYPGAVTVAW KADSSPV AG
VETTTPSKQS NNKYAASSYL SLTPEQWKSH RSYSCQVTHE GSTVEKTVAP TECS
SEQ ID NOS: 229-230 - AMINO ACID SEQUENCES OF A FUSION PROTEIN
COMPRISING BELIMUMAB-BAFFR-TACI
SEQ ID NO: 229 - HEAVY CHAIN
QVQLQQSGAE VKKPGSSVRV SCKASGGTFN NNAINWVRQA PGQGLEWMGG I
IPMFGTAKY
SQNFQGRVAI TADESTGTAS MELSSLRSED TAVYYCARSR DLLLFPHHAL
SPWGRGTMVT
VSSASTKGPS VFPLAPSSKS TSGGTAALGC LVKDYFPEPV TVS NSGALT
SGVHTFPAVL
QSSGLYSLSS VVTVPSSSLG TQTYICNVNH KPSNTKVDKK VEPKSCDKTH
TCPPCPAPEL
LGGPSVFLFP PKPKDTLMI S RTPEVTCVVV DVSHEDPEVK FNWYVDGVEV
HNAKTKPREE
QYNSTYRVVS VLTVLHQDWL NGKEYKCKVS NKALPAPIEK TI SKAKGOPR
EPQVYTLPPS
RDELTKNQVS LTCLVKGFYP SDIAVEWESN GQPENNYKTT PPVLDSDGSF
FLYSKLTVDK
SRWQQGNVFS CSVMHEALHN HYTQKSLSLS PGKGGGGSGGGGS
RGPRSLRGRDAPAPTPCVPAECFDLLVRHCVACGLLRTPRPKPAGASSPAPRTALQPQ
ESVGAGAGEAALPLPGLLGGGGSGGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 230 - LIGHT CHAIN
SSELTQDPAV SVALGQTVRV TCQGDSLRSY YASWYQQKPG QAPVLVIYGK NNRPSGI
PDR FSGSSSGNTA SLTITGAQAE DEADYYCSSR DSSGNHWVFG GGTELTVLGQ
PKAAPSVTLF PPSSEELQAN KATLVCLISD FYPGAVTVAW KADSSPV AG
VETTTPSKQS NNKYAASSYL SLTPEQWKSH RSYSCQVTHE GSTVEKTVAP TECS
SEQ ID NOS: 231-232 - AMINO ACID SEQUENCES OF A FUSION PROTEIN
COMPRISING SECUKINUMAB-SOLUBLE RECEPTOR IL-17RC
SEQ ID NO: 231 - HEAVY CHAIN FUSION
EVQLVESGGG LVQPGGSLRL SCAASGFTFS NYWMNWVRQA PGKGLEWVAA
INQDGSEKYY
VGSVKGRFTI SRDNAKNSLY LQMNSLRVED TAVYYCVRDY YDILTDYYIH
YWYFDLWGRG
TLVTVSSAST KGPSVFPLAP SSKSTSGGTA ALGCLVKDYF PEPVTVSWNS
GALTSGVHTF
PAVLQSSGLY SLSSVVTVPS SSLGTQTYIC NVNHKPSNTK VDKRVEPKSC
DKTHTCPPCP
APELLGGPSV FLFPPKPKDT LMISRTPEVT CVVVDVSHED PEVKFNWYVD
GVEVHNAKTK
PREEQYNSTY RVVSVLTVLH QDWLNGKEYK CKVSNKALPA PIEKTISKAK
GQPREPQVYT
LPPSREEMTK NQVSLTCLVK GFYPSDIAVE WESNGQPENN YKTTPPVLDS
DGSFFLYSKL
TVDKSRWQQG NVFSCSVMHE ALHNHYTQKSGGGGSGGGGS
LERLVGPQDATHCSPVSLEPWGDEERLRVQFLAQQSLSLAPVTAATARTALSGLSGADG
RREERGRGKSWVCLSLGGSGNTEPQKKGLSCRLWDSDILCLPGDIVPAPGPVLAPTHLQ
TELVLRCQKETDCDLCLRVAVHLAVHGHWEEPEDEEKFGGAADSGVEEPRNASLQAQ
VVLSFQAYPTARCVLLEVQVPAALVQFGQSVGSVVYDCFEAALGSEVRIWSYTQPRYE
KELNHTQQLPDCRGLEVWNSIPSCWALPWLNVSADGDNVHLVLNVSEEQHFGLSLYW
NQVQGPPKPRWHKNLTGPQIITLNHTDLVPCLCIQVWPLEPDSVRTNICPFREDPRAHQN
LWQAARLQLLTLQSWLLDAPCSLPAEAALCWRAPGGDPCQPLVPPLSWENVTVDKVL
EFPLLKGHPNLCVQVNSSEKLQLQECLWADSLGPLKDDVLLLETRGPQDNRSLCALEPS
GCTSLPSKASTRAARLGEYLLQDLQSGQCLQLWDDDLGALWACPMDKYIHKR
SEQ ID NO: 232 - LIGHT CHAIN
EIVLTQSPGT LSLSPGERAT LSCRASQSVS SSYLAWYQQK PGQAPRLLIY
GASSRATGIP
DRFSGSGSGT DFTLTISRLE PEDFAVYYCQ QYGSSPCTFG QGTRLEIKRT VAAPSVFIFP
PSDEQLKSGT ASVVCLLNNF YPREAKVQWK VDNALQSGNS QESVTEQDSK
DSTYSLSSTL
TLSKADYEKH KVYACEVTHQ GLSSPVTKSF NRGEC
SEQ ID NOS: 233-234 AMINO ACID SEQUENCES OF A FUSION PROTEIN
COMPRISING TEPLIZUMAB-LINKER-TACI
SEQ ID NO: 233 - HEAVY CHAIN FUSION
QVQLVQSGGG VVQPGRSLRL SCKASGYTFT RYTMHWVRQA PGKGLEWIGY
INPSRGYTNY
NQKVKDRFTI SRDNSKNTAF LQMDSLRPED TGVYFCARYY DDHYCLDYWG
QGTPVTVSSA
STKGPSVFPL APSSKSTSGG TAALGCLVKD YFPEPVTVSW NSGALTSGVH
TFPAVLQSSG
LYSLSSVVTV PSSSLGTQTY ICNVNHKPSN TKVDKKVEPK SCDKTHTCPP
CPAPEAAGGP
SVFLFPPKPK DTLMISRTPE VTCVVVDVSH EDPEVKFNWY VDGVEVHNAK
TKPREEQYNS
TYRVVSVLTV LHQDWLNGKE YKCKVSNKAL PAPIEKTISK AKGQPREPQV
YTLPPSRDEL
TKNQVSLTCL VKGFYPSDIA VEWESNGQPE NNYKTTPPVL DSDGSFFLYS
KLTVDKSRWQ
QGNVFSCSVM HEALHNHYTQ KSLSLSPGK GGGGSGGGGS
SGLGRSRRGGRSRVDQEERFPQGLWTGVAMRSCPEEQYWDPLLGTCMSCKTICNHQSQ
RTCAAFCRSLSCRKEQGKFYDHLLRDCISCASICGQHPKQCAYFCENKLRSPVNLPPELR
RQRSGEVENNSDNSGRYQGLEHRGSEASPALPGLKLSADQVALVYS
SEQ ID NO: 234 - LIGHT CHAIN
DIQMTQSPSS LSASVGDRVT ITCSASSSVS YMNWYQQTPG KAPKRWIYDT
SKLASGVPSR
FSGSGSGTDY TFTISSLQPE DIATYYCQQW SSNPFTFGQG TKLQITRTVA APSVFIFPPS
DEQLKSGTAS VVCLLNNFYP REAKVQWKVD NALQSGNSQE SVTEQDSKDS
TYSLSSTLTL
SKADYEKHKV YACEVTHQGL SSPVTKSFNR GEC
SEQ ID NOS: 235-236 AMINO ACID SEQUENCES OF A FUSION PROTEIN
COMPRISING LECANEMAB -FC-IL-10
SEQ ID NO: 235 - HEAVY CHAIN FUSION
EVQLVESGGG LVQPGGSLRL SCSASGFTFS SFGMHWVRQA PGKGLEWVAY
ISSGSSTIYY
GDTVKGRFTI SRDNAKNSLF LQMSSLRAED TAVYYCAREG GYYYGRSYYT
MDYWGQGTTVTVSSASTKGP SVFPLAPSSK STSGGTAALG CLVKDYFPEP
VTVSWNSGAL TSGVHTFPAVLQSSGLYSLS SVVTVPSSSL GTQTYICNVN
HKPSNTKVDK RVEPKSCDKT HTCPPCPAPELLGGPSVFLF PPKPKDTLMI
SRTPEVTCVV VDVSHEDPEV KFNWYVDGVE VHNAKTKPREEQYNSTYRVV
SVLTVLHQDW LNGKEYKCKV SNKALPAPIE KTISKAKGQP
REPQVYTLPPSREEMTKNQV SLTCLVKGFY PSDIAVEWES NGQPENNYKT
TPPVLDSDGS FFLYSKLTVDKSRWQQGNVF SCSVMHEALH NHYTQKSLSL
SPGKGGGGSGGGGS
SPGQGTQSENSCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCHRFLPCENKS
KAVEQVKNAFNKLQEKGIYKAMSEFDIFINYIEAYMTMKIRN
SEQ ID NO: 236 - LIGHT CHAIN
DVVMTQSPLS LPVTPGAPAS ISCRSSQSIV HSNGNTYLEW YLQKPGQSPK
LLIYKVSNRF
SGVPDRFSGS GSGTDFTLRI SRVEAEDVGI YYCFQGSHVP PTFGPGTKLE
IKRTVAAPSV
FIFPPSDEQL KSGTASVVCL LNNFYPREAK VQWKVDNALQ SGNSQESVTE
QDSKDSTYSL
SSTLTLSKAD YEKHKVYACE VTHQGLSSPV TKSFNRGEC
SEQ ID NO: 237 - AMINO ACID SEQUENCE OF A FUSION PROTEIN
COMPRISING IL-2 -Fc -linker- BAFFR
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSREEMTK NQVSLTCLVK GFYPSDIAVE WESNGQPENN
YKTTPPVLDS
DGSFFLYSKL TVDKSRWQQG NVFSCSVMHE ALHNHYTQKS LSLSPGKGGGSGGGS
RGPRSLRGRDAPAPTPCVPAECFDLLVRHCVACGLLRTPRPKPAGASSPAPRTALQPQES
VGAGAGEAALPLPGLL
SEQ ID NO: 238 - AMINO ACID SEQUENCE OF A FUSION PROTEIN
COMPRISING IL2 -Fc -linker- BAFFR -linker BAFFR
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
DKTHTCPPCP APELLGGPSV FLFPPKPKDT LMISRTPEVT CVVVDVSHED
PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK
GFYPSDIAVE WESNGQPENN YKTTPPVLDS DGSFFLYSKL TVDKSRWQQG
NVFSCSVMHE ALHNHYTQKS LSLSPGK
RGPRSLRGRDAPAPTPCVPAECFDLLVRHCVACGLLRTPRPKPAGASSPAPRTALQPQES
VGAGAGEAALPLPGLL
GGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 239 - AMINO ACID SEQUENCES OF A FUSION PROTEIN
COMPRISING IL10 -Fc -linker- TNFR2
SPGQGTQSENSCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCHRFLPCENKS
KAVEQVKNAFNKLQEKGIYKAMSEFDIFINYIEAYMTMKIRN
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSREEMTK NQVSLTCLVK GFYPSDIAVE WESNGQPENN
YKTTPPVLDS
DGSFFLYSKL TVDKSRWQQG NVFSCSVMHE ALHNHYTQKS LSLSPGKGGGSGGGS
LPAQVAFTPY APEPGSTCRL REYYDQTAQM CCSKCSPGQH AKVFCTKTSD
TVCDSCEDSTYTQLWNWVPE CLSCGSRCSS DQVETQACTR EQNRICTCRP
GWYCALSKQE GCRLCAPLRKCRPGFGVARP GTETSDVVCK PCAPGTFSNT
TSSTDICRPH QICNVVAIPG NASMDAVCTSTSPTRSMAPG AVHLPQPVST
RSQHTQPTPE PSTAPSTSFL LPMGPSPPAE GSTGD
SEQ ID NO: 240 - AMINO ACID SEQUENCES OF A FUSION PROTEIN
COMPRISING IL10 -Fc -LINKER- IL17RA
SPGQGTQSENSCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCHRFLPCENKS
KAVEQVKNAFNKLQEKGIYKAMSEFDIFINYIEAYMTMKIRN
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSREEMTK NQVSLTCLVK GFYPSDIAVE WESNGQPENN
YKTTPPVLDS
DGSFFLYSKL TVDKSRWQQG NVFSCSVMHE ALHNHYTQKS
LSLSPGKGGGSGGGSGSSSS
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLW
SEQ ID NO: 241 - AMINO ACID SEQUENCE OF A FUSION PROTEIN
COMPRISING IL10 -Fc -linker- BAFFR-LINKER-BAFFR
SPGQGTQSENSCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCHRFLPCENKS
KAVEQVKNAFNKLQEKGIYKAMSEFDIFINYIEAYMTMKIRN
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSREEMTK NQVSLTCLVK GFYPSDIAVE WESNGQPENN
YKTTPPVLDS
DGSFFLYSKL TVDKSRWQQG NVFSCSVMHE ALHNHYTQKS
LSLSPGKGGGSGGGSGSSSS
RGPRSLRGRDAPAPTPCVPAECFDLLVRHCVACGLLRTPRPKPAGASSPAPRTALQPQES
VGAGAGEAALPLPGLLGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 242 - AMINO ACID SEQUENCES OF A FUSION PROTEIN
COMPRISING IL10 -Fc -LINKER- IL-2
SPGQGTQSENSCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCHRFLPCENKS
KAVEQVKNAFNKLQEKGIYKAMSEFDIFINYIEAYMTMKIRN
DKTHTCPPCP APELLGGPSV FLFPPKPKDT LMISRTPEVT CVVVDVSHED
PEVKFNWYVD
GVEVHNAKTK PREEQYNSTY RVVSVLTVLH QDWLNGKEYK CKVSNKALPA
PIEKTISKAK
GQPREPQVYT LPPSREEMTK NQVSLTCLVK GFYPSDIAVE WESNGQPENN
YKTTPPVLDS
DGSFFLYSKL TVDKSRWQQG NVFSCSVMHE ALHNHYTQKS LSLSPGKGGGSGGGS
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
SEQ ID NO: 243 - AMINO ACID SEQUENCE OF A FUSION PROTEIN
COMPRISING IL17RA-LINKER- AN ALBUMIN BINDING SITE-LINKER
BAFFR
LRLLDHRALVCSQPGLNCTVKNSTCLDDSWIHPRNLTPSSPKDLQIQLHFAHTQQGDLFP
VAHIEWTLQTDASILYLEGAELSVLQLNTNERLCVRFEFLSKLRHHHRRWRFTFSHFVV
DPDQEYEVTVHHLPKPIPDGDPNHQSKNFLVPDCEHARMKVTTPCMSSGSLWDPNITVE
TLEAHQLRVSFTLWNESTHYQILLTSFPHMENHSCFEHMHHIPAPRPEEFHQRSNVTLTL
RNLKGCCRHQVQIQPFFSSCLNDCLRHSATVSCPEMPDTPEPIPDYMPLWGGGGSGGGG
S GGGGS
GVSDFYKKLIEKAKTVEGVEALKDAI
GGGGSGGGGSGGGGS
RGPRSLRGRDAPAPTPCVPAECFDLLVRHCVACGLLRTPRPKPAGASSPAPRTALQPQES
VGAGAGEAALPLPGLL
SEQ ID NO: 244 - AMINO ACID SEQUENCE OF A FUSION PROTEIN
COMPRISING CD40 - AN ALBUMIN BINDING SITE-LINKER BAFFR-
LINKER- BAFFR
EPPTACREKQYLINSQCCSLCQPGQKLVSDCTEFTETECLPCGESEFLDTWNRETHCHQH
KYCDPNLGLRVQQKGTSETDTICTCEEGWHCTSEACESCVLHRSCSPGFGVKQIATGVS
DTICEPCPVGFFSNVSSAFEKCHPWTSCETKDLVVQQAGTNKTDVVCGPQDRLR
GVSDFYKKLIEKAKTVEGVEALKDAI
GGGGSGGGGSGGGGS
RGPRSLRGRDAPAPTPCVPAECFDLLVRHCVACGLLRTPRPKPAGASSPAPRTALQPQES
VGAGAGEAALPLPGLL GGGGSGGGGSGGGGS
RGPRSLRGRDAPAPTPCVPAECFDLLVRHCVACGLLRTPRPKPAGASSPAPRTALQPQES
VGAGAGEAALPLPGLL
AMINO ACID SEQUENCES OF FUSION PROTEINS COMPRISING A BISPECIFIC
ANTIBODY WITH CYTOKINES
SEQ ID NOS: 245 - 248 - FUSION PROTEIN OF HEAVY CHAINS OF
TECLISTAMAB WITH HUMAN IL-2
SEQ ID NO: 245 - HEAVY CHAIN A FUSION
QLQLQESGPG LVKPSETLSL TCTVSGGSIS SGSYFWGWIR QPPGKGLEWI
GSIYYSGITY
YNPSLKSRVT ISVDTSKNQF SLKLSSVTAA DTAVYYCARH DGAVAGLFDY
WGQGTLVTVS
SASTKGPSVF PLAPCSRSTS ESTAALGCLV KDYFPEPVTV SWNSGALTSG
VHTFPAVLQS
SGLYSLSSVV TVPSSSLGTK TYTCNVDHKP SNTKVDKRVE SKYGPPCPPC
PAPEAAGGPS
VFLFPPKPKD TLMISRTPEV TCVVVDVSQE DPEVQFNWYV DGVEVHNAKT
KPREEQFNST
YRVVSVLTVL HQDWLNGKEY KCKVSNKGLP SSIEKTISKA KGQPREPQVY
TLPPSQEEMT
KNQVSLTCLV KGFYPSDIAV EWESNGQPEN NYKTTPPVLD SDGSFFLYSR
LTVDKSRWQE
GNVFSCSVMH EALHNHYTQK SLSLSLGK
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFL
NRWITFCQSIISTLT
SEQ ID NO: 246 - LIGHT CHAIN A
SYVLTQPPSV SVAPGQTARI TCGGNNIGSK SVHWYQQPPG QAPVVVVYDD
SDRPSGIPER
FSGSNSGNTA TLTISRVEAG DEAVYYCQVW DSSSDHVVFG GGTKLTVLGQ
PKAAPSVTLF
PPSSEELQAN KATLVCLISD FYPGAVTVAW KGDSSPVKAG VETTTPSKQS
NNKYAASSYL
SLTPEQWKSH RSYSCQVTHE GSTVEKTVAP TECS
SEQ ID NO: 247 - HEAVY CHAIN B FUSION
EVQLVESGGG LVQPGGSLRL SCAASGFTFN TYAMNWVRQA PGKGLEWVAR
IRSKYNNYAT
YYAASVKGRF TISRDDSKNS LYLQMNSLKT EDTAVYYCAR HGNFGNSYVS
WFAYWGQGTL
VTVSSASTKG PSVFPLAPCS RSTSESTAAL GCLVKDYFPE PVTVSWNSGA
LTSGVHTFPA
VLQSSGLYSL SSVVTVPSSS LGTKTYTCNV DHKPSNTKVD KRVESKYGPP
CPPCPAPEAA
GGPSVFLFPP KPKDTLMISR TPEVTCVVVD VSQEDPEVQF NWYVDGVEVH
NAKTKPREEQ
FNSTYRVVSV LTVLHQDWLN GKEYKCKVSN KGLPSSIEKT ISKAKGQPRE
PQVYTLPPSQ
EEMTKNQVSL TCLVKGFYPS DIAVEWESNG QPENNYKTTP PVLDSDGSFL
LYSKLTVDKS
RWQEGNVFSC SVMHEALHNH YTQKSLSLSL GK
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFL
NRWITFCQSIISTLT
SEQ ID NO: 248 - LIGHT CHAIN B
QTVVTQEPSL TVSPGGTVTL TCRSSTGAVT TSNYANWVQQ KPGQAPRGLI
GGTNKRAPGT
PARFSGSLLG GKAALTLSGV QPEDEAEYYC ALWYSNLWVF GGGTKLTVLG
QPKAAPSVTL
FPPSSEELQA NKATLVCLIS DFYPGAVTVA WKADSSPVKA GVETTTPSKQ
SNNKYAASSY
LSLTPEQWKS HRSYSCQVTH EGSTVEKTVA PTECS
SEQ ID NOS: 249-252 FUSION PROTEIN OF BOTH HEAVY CHAINS OF
TECLISTAMAB WITH HUMAN INTERFERON GAMMA
SEQ ID NO: 249 - HEAVY CHAIN A FUSION
QLQLQESGPG LVKPSETLSL TCTVSGGSIS SGSYFWGWIR QPPGKGLEWI
GSIYYSGITY
YNPSLKSRVT ISVDTSKNQF SLKLSSVTAA DTAVYYCARH DGAVAGLFDY
WGQGTLVTVS
SASTKGPSVF PLAPCSRSTS ESTAALGCLV KDYFPEPVTV SWNSGALTSG
VHTFPAVLQS
SGLYSLSSVV TVPSSSLGTK TYTCNVDHKP SNTKVDKRVE SKYGPPCPPC
PAPEAAGGPS
VFLFPPKPKD TLMISRTPEV TCVVVDVSQE DPEVQFNWYV DGVEVHNAKT
KPREEQFNST
YRVVSVLTVL HQDWLNGKEY KCKVSNKGLP SSIEKTISKA KGQPREPQVY
TLPPSQEEMT
KNQVSLTCLV KGFYPSDIAV EWESNGQPEN NYKTTPPVLD SDGSFFLYSR
LTVDKSRWQE
GNVFSCSVMHEALHNHYTQKSLSLSLGKQDPYVKEAENLKKYFNAGHSDVADNGTLF
LGILKNWKEESDRKIMQSQIVSFYFKLFKNFKDDQSIQKSVETIKEDMNVKFFNSNKKK
RDDFEKLTNYSVTDLNVQRKAIHELIQVMAELSPAAKTGKRKRSQMLFRGRRASQ
SEQ ID NO: 250 - LIGHT CHAIN A
SYVLTQPPSV SVAPGQTARI TCGGNNIGSK SVHWYQQPPG QAPVVVVYDD
SDRPSGIPER
FSGSNSGNTA TLTISRVEAG DEAVYYCQVW DSSSDHVVFG GGTKLTVLGQ
PKAAPSVTLF
PPSSEELQAN KATLVCLISD FYPGAVTVAW KGDSSPVKAG VETTTPSKQS
NNKYAASSYL
SLTPEQWKSH RSYSCQVTHE GSTVEKTVAP TECS
SEQ ID NO: 251 - HEAVY CHAIN B FUSION
EVQLVESGGG LVQPGGSLRL SCAASGFTEN TYAMNWVRQA PGKGLEWVAR
IRSKYNNYAT YYAASVKGRF TISRDDSKNS LYLQMNSLKT EDTAVYYCAR
HGNFGNSYVS WFAYWGQGTL VTVSSASTKG PSVFPLAPCS RSTSESTAAL
GCLVKDYFPE PVTVSWNSGA LTSGVHTFPA VLQSSGLYSL SSVVTVPSSS
LGTKTYTCNV DHKPSNTKVD KRVESKYGPP CPPCPAPEAA GGPSVFLFPP
KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE
PQVYTLPPSQ EEMTKNQVSL TCLVKGFYPS DIAVEWESNG QPENNYKTTP
PVLDSDGSFL LYSKLTVDKS RWQEGNVFSC SVMHEALHNH YTQKSLSLSL
GKQDPYVKEAENLKKYFNAGHSDVADNGTLFL
GILKNWKEESDRKIMQSQIVSFYFKLFKNFKDDQSIQKSVETIKEDMNVKFFNSNKKKR
DDFEKLT NYSVTDLNVQRKAIHELIQVMAELSPAAKTGKRKRSQMLFRGRRASQ
SEQ ID NO: 252 - LIGHT CHAIN
QTVVTQEPSL TVSPGGTVTL TCRSSTGAVT TSNYANWVQQ KPGQAPRGLI
GGTNKRAPGT PARFSGSLLG GKAALTLSGV QPEDEAEYYC ALWYSNLWVF
GGGTKLTVLG QPKAAPSVTL FPPSSEELQA NKATLVCLIS DFYPGAVTVA
WKADSSPVKA GVETTTPSKQ SNNKYAASSY LSLTPEQWKS HRSYSCQVTH
EGSTVEKTVA PTECS

What is claimed is:

1. A fusion polypeptide, comprising:
at least two ligand binding domains, each ligand binding domain having an amino acid sequence selected from the group consisting of SEQ ID NOS: 1-25 or a fragment or variant thereof; and
a fragment crystallizable (Fc) region of immunoglobulin G (IgG) or a fragment or variant thereof.

2. The fusion polypeptide of claim 1, wherein the fusion polypeptide comprises two ligand binding domains.

3. The fusion polypeptide of claim 1, wherein the fusion polypeptide comprises three ligand binding domains.

4. The fusion polypeptide of claim 1, wherein each of the at least two ligand binding domains are different.

5. The fusion polypeptide of claim 1, wherein the fusion polypeptide comprises at least three ligand binding domains, and wherein at least two of the ligand binding domains are the same.

6. The fusion polypeptide of claim 1, wherein the Fc region comprises has an amino acid sequence selected from the group consisting of SEQ ID NOS: 26-42.

7. The fusion polypeptide of claim 1, further comprising a linker peptide for connecting the at least two ligand binding domains to each other and/or to the Fc region.

8. The fusion polypeptide of claim 7, wherein the linker polypeptide is selected from the group consisting of SEQ ID NOS: 43-71.

9. The fusion polypeptide of claim 1, wherein the fusion polypeptide comprises a sequence selected from the group consisting of SEQ ID NOS: 72-182.

10. The fusion polypeptide of claim 1, wherein the fusion polypeptide comprises a sequence selected from the group consisting of SEQ ID NOS: 102-119.

11. The fusion polypeptide of claim 1, wherein the fusion polypeptide comprises a sequence selected from the group consisting of SEQ ID NOS: 73-74 and 118-119.

12. The fusion polypeptide of claim 1, wherein the fusion polypeptide comprises the sequence of SEQ ID NO: 120.

13. The fusion polypeptide of claim 7, wherein the fusion polypeptide comprises a sequence selected from the group consisting of SEQ ID NOS: 72-124.

14. The fusion polypeptide of claim 1, wherein the ligand binding domain comprises a BAFFR selected from the group consisting of SEQ ID NOS: 2-8.

15. The fusion polypeptide of claim 14, wherein the fusion protein comprises two or more BAFFR selected from the group consisting of SEQ ID NOS: 2-8.

16. The fusion polypeptide of claim 1, wherein the ligand binding domain comprises an IL-17 Receptor having the sequence of SEQ ID NO: 1.

17. The fusion polypeptide of claim 1, wherein the ligand binding domain comprises an IL-21 Receptor selected from the group consisting of SEQ ID NO: 16 and 24.

18. The fusion polypeptide of claim 1, wherein the ligand binding domain comprises a TNF Receptor selected from the group consisting of SEQ ID NOS: 10-12 and 19.

19. The fusion polypeptide of claim 1, wherein the ligand binding domain comprises a IFN Receptor selected from the group consisting of SEQ ID NOS: 9 and 14-15.

20. A fusion polypeptide, comprising the sequence of SEQ ID NO: 121.

21. A fusion polypeptide, comprising the sequence of SEQ ID NO: 122.

22. A fusion polypeptide, comprising an Fc region having a sequence selected from the group consisting of SEQ ID NOS: 26-42.

23. The fusion polypeptide of claim 22, wherein the Fc region has a sequence selected from the group consisting of SEQ ID NOS: 29 or 30.

24. The fusion polypeptide of claim 22, wherein the Fc region comprises the sequence of SEQ ID NO: 37.

25. The fusion polypeptide of claim 22, wherein the Fc region has a sequence selected from the group consisting of SEQ ID NOS: 26-41.

26. The fusion polypeptide of claim 1, wherein the at least two ligand binding domains comprises at least one ligand binding domain that reduces B cell activity and at least one ligand binding domain that reduces T cell activity.

27. The fusion polypeptide of claim 26, wherein the at least one ligand binding domain that reduces B cell activity and/or the at least one ligand binding domain that reduces T cell activity slows the progression of Sjogren's Syndrome.

28. The fusion polypeptide of claim 1, wherein the fusion polypeptide comprises a sequence selected from the group consisting of SEQ ID NOS: 183-252.

29. An isolated nucleic acid sequence encoding a fusion polypeptide including
at least two ligand binding domains, each ligand binding domain having an amino acid sequence selected from the group consisting of SEQ ID NOS: 1-25 or a fragment or variant thereof, and
a fragment crystallizable (Fc) region of immunoglobulin G (IgG) or a fragment or variant thereof.

30. The isolated nucleic acid sequence of claim 29, wherein the nucleic acid encodes a fusion polypeptide having a sequence selected from the group consisting of SEQ ID NOS: 72-182.

31. A vector comprising the isolated nucleic acid sequence of claim 29.

32. The vector of claim 31, wherein the isolated nucleic acid is operatively linked to an expression cassette.

33. An isolated cell comprising a nucleic acid sequence encoding a fusion polypeptide including
at least two ligand binding domains, each ligand binding domain having an amino acid sequence selected from the group consisting of SEQ ID NOS: 1-25 or a fragment or variant thereof, and
a fragment crystallizable (Fc) region of immunoglobulin G (IgG) or a fragment or variant thereof.

34. A pharmaceutical composition, comprising a fusion polypeptide including
at least two ligand binding domains, each ligand binding domain having an amino acid sequence selected from the group consisting of SEQ ID NOS:1-25 or a fragment or variant thereof,
a fragment crystallizable (Fc) region of immunoglobulin G (IgG) or a fragment or variant thereof,
and a pharmaceutically-acceptable vehicle, carrier, or excipient.

35. A method of treating Sjögren's Syndrome and/or Systemic Lupus Erythematosus, comprising administering to a subject in need thereof a fusion polypeptide including
at least two ligand binding domains, each ligand binding domain having an amino acid sequence selected from the group consisting of SEQ ID NOS: 1-25 or a fragment or variant thereof, and
a fragment crystallizable (Fc) region of immunoglobulin G (IgG) or a fragment or variant thereof.

36. A fusion polypeptide, comprising a monoclonal antibody binding domain for binding CD20 and a ligand binding domain from PD1.

37. A fusion polypeptide, comprising a monoclonal antibody binding domain for binding CD20 and one, two, or three ligand binding domains for binding BAFF.

38. A fusion polypeptide, comprising a monoclonal antibody binding domain for binding CD20 and one, two, or three ligand binding domains from TACI.

39. A fusion polypeptide, comprising a monoclonal antibody binding domain for binding CD20, one, two, or three ligand binding domain for binding BAFF, and a ligand binding domain from BCMA.

40. A fusion polypeptide, comprising a monoclonal antibody binding domain for binding BAFF, a ligand binding domain for binding BAFF, and a ligand binding domain from TACI.

41. A fusion polypeptide, comprising a monoclonal antibody binding domain for binding BAFF and a ligand binding domain from BCMA.

42. A fusion polypeptide comprising the sequences of SEQ ID NOS: 203-204.

43. A fusion polypeptide comprising the sequences of SEQ ID NOS: 223-224.

44. A fusion polypeptide comprising the sequences of SEQ ID NOS: 225-226.

45. A fusion polypeptide comprising the sequences of SEQ ID NOS: 227-228.

46. A fusion polypeptide comprising the sequences of SEQ ID NOS: 229-230.

47. A fusion polypeptide comprising the sequences of SEQ ID NOS: 231-232.

48. A fusion polypeptide comprising the sequences of SEQ ID NOS: 233-234.

49. A fusion polypeptide comprising the sequences of SEQ ID NOS: 235-236.

50. A fusion polypeptide comprising the sequence of SEQ ID NO: 237.

51. A fusion polypeptide comprising the sequence of SEQ ID NO: 238.

52. A fusion polypeptide comprising the sequence of SEQ ID NO: 239.

53. A fusion polypeptide comprising the sequence of SEQ ID NO: 240.

54. A fusion polypeptide comprising the sequence of SEQ ID NO: 241.

55. A fusion polypeptide comprising the sequence of SEQ ID NO: 242.

56. A fusion polypeptide comprising the sequence of SEQ ID NO: 243.

57. A fusion polypeptide comprising the sequence of SEQ ID NO: 244.

58. A fusion polypeptide comprising the sequences of SEQ ID NOS: 245-248.

59. A fusion polypeptide comprising the sequences of SEQ ID NOS: 249-252.

60. A fusion polypeptide, comprising:
at least two ligand binding domains, where one of the at least two ligand binding domains binds modulates B cell activity and the other of the at least two ligand binding domains modulates T cell activity; and
a fragment crystallizable (Fc) region of immunoglobulin G (IgG) or a fragment or variant thereof.

61. A method of treating a subject having a mutation in an amino acid sequence or nucleic acid sequence encoding BAFF, comprising administering to the subject an effective amount of the fusion polypeptide of claim 60.

62. The method of claim 61, wherein the subjects have a biomarker selected from: an ANA titer greater than 1:80; and anti-dsDNA greater than 30 IU/ml; an anti-Sm greater than 15 units/ml; a C3 less than 900 mg/liter; a C4 less than 60 mg/liter; and a CRP positive greater than 3 mg/ml.

63. The method of claim 61, wherein the subject has or is at risk of developing Systemic Lupus Erythematosus flares.
